(19) United States
(12) Patent Application Publication
(10) Pub. No.: US 2020/0399264 A1
(43) Pub. Date: Dec. 24, 2020

(54) 4-SUBSTITUTED-2-THIAZOLE AMIDES AS ANTIVIRAL AGENTS

(71) Applicants: Southern Research Institute, Birmingham, AL (US); Oregon Health and Science University, Portland, OR (US)

(72) Inventors: Ashish Kumar Pathak, Birmingham, AL (US); Corinne E. Augelli-Szafran, Homewood, AL (US); Syed Kaleem Ahmed, Hoover, AL (US); Atefeh Garzan, Hoover, AL (US); Daniel Streblow, Banks, OR (US); Nicole Haese, Beaverton, OR (US)

(21) Appl. No.: 16/902,080

(22) Filed: Jun. 15, 2020

Related U.S. Application Data
(60) Provisional application No. 62/864,288, filed on Jun. 20, 2019.

(51) Int. Cl. C07D 417/14; C07D 417/12; C07D 263/48

(52) U.S. Cl. CPC C07D 417/14; C07D 263/48; C07D 417/12

(57) ABSTRACT

The present disclosure is concerned with benzoannulene compounds that are capable of inhibiting a viral infection and methods of treating viral infections such as, for example, chikungunya, Venezuelan equine encephalitis, Eastern equine encephalitis, Western equine encephalitis, dengue, West Nile, influenza, and zika, using these compounds. This abstract is intended as a scanning tool for purposes of searching in the particular art and is not intended to be limiting of the present invention.

### CROSS-REFERENCE TO RELATED APPLICATIONS

This application claims the benefit of U.S. Provisional Application No. 62/864,288, filed on Jun. 20, 2019, the contents of which are hereby incorporated by reference in their entirety.

### STATEMENT REGARDING FEDERALLY SPONSORED RESEARCH

This invention was made with government support under grant numbers 1U19AI109680 and 1U19AI142759 awarded by the National Institutes of Health (NIH). The government has certain rights in the invention.

### BACKGROUND

Arthropod-borne viruses have developed a complex life cycle adapted to alternate between insect and vertebrate hosts. These arthropod-borne viruses belong mainly to the families Togaviridae, Flaviviridae, and Bunyaviridae. Flavivirus is a genus of the family Flaviviridae. This genus includes such viral infections as West Nile virus, dengue virus (DENV), Tick-borne Encephalitis virus, yellow fever virus, and zika virus (ZIKV) that may cause encephalitis. Alphavirus is a genus of the family Togaviridae. This genus includes such viral infections as chikungunya virus (CHIKV), Venezuelan Equine Encephalitis virus (VEEV), Western Equine Encephalitis virus (WEEV), and Eastern Equine Encephalitis virus (EEEV).

Zika virus (ZIKV) is a single stranded RNA virus transmitted to humans primarily via Aedes aegypti mosquitos and other mosquitos of the Stegomyia subgenus. ZIKV can also be transmitted through sexual intercourse, a blood transfusion, or from a pregnant woman to her fetus. Infection during pregnancy can result in microcephaly and other severe fetal brain defects. Additional problems detected among fetuses and infants infected with ZIKV before birth include as defects of the eye, hearing deficits, and impaired growth. Increased reports of Guillain-Barre syndrome have also been observed in areas affected by Zika. Until recently, only sporadic human ZIKV infections had been reported. Since 2007, ZIKV has expanded from Asia and Africa to include both North and South America.

DENV is a mosquito-borne virus estimated to cause 50-100 million infections each year. DENV infections can result in serious diseases including dengue fever, dengue hemorrhagic fever, and dengue shock syndrome, and may even result in death. This virus is considered by the World Health Organization to be the most important mosquito-borne viral disease worldwide.

Originally isolated in Tanzania, sporadic outbreaks of CHIKV have continued to plague Asia and Africa. In 2007, the first outbreak in Europe was documented with over 200 confirmed cases. To date, CHIKV has been identified in over 40 countries including the United States of America. The symptoms of CHIKV, which include fever, rash, and severe joint pain, are commonly indistinguishable from ZIKV and DENV. While most patients usually recover after days to weeks, some may develop chronic arthritis. Additionally, death related to Chikungunya infection has been reported in older patients or patients with weakened immune systems.

Currently, there are no approved treatments for ZIKV, DENV, VEEV, EEEV, WEEV, or CHIKV. Despite the widespread distribution and severity of the effects of these viral infections, a treatment for ZIKV, DENV, VEEV, EEEV, WEEV and CHIKV has remained elusive. Thus, there remains a need for antiviral agents capable of targeting these viruses and methods of making and using same.

### SUMMARY

In accordance with the purpose(s) of the invention, as embodied and broadly described herein, the invention, in one aspect, relates to compositions and methods for use in the prevention and treatment of viral infections such as, for example, chikungunya (CHIKV), Venezuelan equine encephalitis (VEEV), Western Equine Encephalitis virus (WEEV), Eastern Equine Encephalitis virus (EEEV), dengue viruses (DENV), West Nile virus (WNV), influenza, and zika virus (ZIKV).

Disclosed are compounds having a structure represented by a formula:

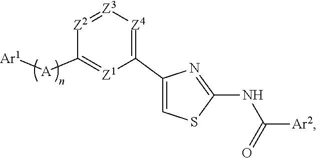

wherein n is selected from 0 and 1; wherein A, when present, is selected from O, NH, N(C1-C4 alkyl), and N(Ar³); wherein Ar³, when present, is phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of Z¹, Z², Z³, and Z⁴ is independently selected from N and CR¹⁰, provided that at least two of Z¹, Z², Z³, and Z⁴ are CR¹⁰; wherein each occurrence of R¹⁰, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², —SO₂R²³, and Cy¹; wherein each occurrence of R²⁰, R²¹, R²², and R²³, when present, is independently selected from hydrogen and C1-C4 alkyl; wherein Cy¹, when present, is selected from 5-membered heterocycle and 6-membered heterocycle, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl; and either: (a) wherein Ar¹ is selected from pyridinyl, piperazinyl, and 6-membered aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴; wherein each occurrence of R¹, R¹², R¹³, and R¹⁴, when present, is independently selected from hydrogen and C1-C4 alkyl; and wherein Ar² is selected from 5-membered heterocycle, 6-membered heterocycle, 5-membered heteroaryl, and 6-membered heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸; and wherein each occurrence of R, R¹⁶, R¹⁷, and R¹⁸, when present, is independently selected from hydrogen and C1-C4 alkyl; or (b) wherein Ar¹ is a structure represented by a formula:

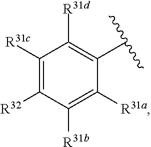

wherein each of R^{31a}, R^{31b}, R^{31c}, and R^{31d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴; wherein R³² is selected from —CN and —CH₂NH₂; and wherein Ar² is selected from 5-membered heterocycle, 6-membered heterocycle, 5-membered heteroaryl, 6-membered heteroaryl, 6-membered aryl, and bicyclo[1.1.1]pentanyl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸, or a pharmaceutically acceptable salt thereof.

Also disclosed are pharmaceutical compositions comprising a therapeutically effective amount of at least one disclosed compound and a pharmaceutically acceptable carrier.

Also disclosed are methods for the treatment of a viral infection in a subject having the viral infection, the method comprising the step of administering to the subject a therapeutically effective amount of at least one disclosed compound.

Also disclosed are kits comprising at least one disclosed compound and one or more of: (a) at least one antiviral agent; (b) a instructions for administering the at least one compound in connection with treating a viral infection; (c) instructions for administering the at least one compound in connection with reducing the risk of viral infection; and (d) instructions for treating a viral infection.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated 10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, “CC₅₀,” is intended to refer to the cell cytotoxicity to the concentration of a substance (e.g., a compound or a drug) that is required for 50% cell survival in a cell viability assay in vitro.

As used herein, “EC₉₀,” is intended to refer to the concentration of a substance (e.g., a compound or a drug) that is required for 90% agonism of a biological process, or component of a process, including a protein, subunit, organelle, ribonucleoprotein, etc. In one aspect, an EC₉₀ can refer to the concentration of a substance that is required for 90% agonism in vivo, as further defined elsewhere herein. In a further aspect, EC₉₀ refers to the concentration of agonist that provokes a response halfway between the baseline and maximum response.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene 9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form, which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, and amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions that can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ—OA³, where “a” is an integer of from 1 to 200 and A, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A₂)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbomenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the 1 clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The terms “pseudohalide,” “pseudohalogen,” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl”, “heteroaryl”, “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula —OH.

The term “ketone” as used herein is represented by the formula AC(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “R,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR*, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR*, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C1-6 aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon-containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

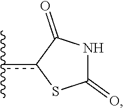

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radioactively labeled forms, isomers, solvates and combinations thereof. The “combinations” mentioned in this context are refer to derivatives falling within at least two of the groups: pharmaceutically acceptable salts, prodrugs, deuterated forms, radioactively labeled forms, isomers, and solvates. Examples of radioactively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically labeled or isotopically substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules that owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

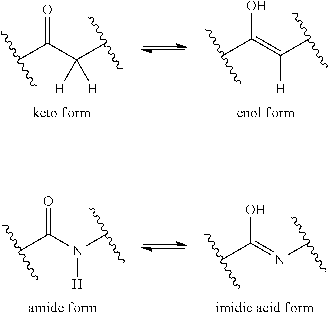 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N-unsubstituted, 5-A³ as shown below.

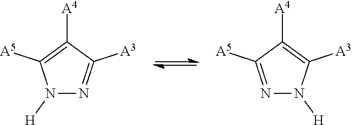

Unless stated to the contrary, the invention includes all such possible tautomers.

It is known that chemical substances form solids that are present in different states of order that are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

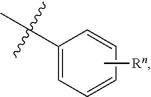

which is understood to be equivalent to a formula:

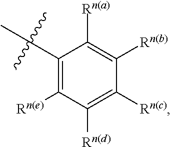

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance Rₙ₍ₐ₎ is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, Mass.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. COMPOUNDS

In one aspect, the invention relates to compounds useful in treating disorders associated with a viral infection, in particular, CHIKV, VEEV, DENV, WNV, influenza, and ZIKV.

In one aspect, the disclosed compounds exhibit antiviral activity.

In one aspect, the compounds of the invention are useful in inhibiting viral activity in a mammal. In a further aspect, the compounds of the invention are useful in inhibiting viral activity in at least one cell.

In one aspect, the compounds of the invention are useful in the treatment of viral infections, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

1. Structure

In one aspect, disclosed are compounds having a structure represented by a formula:

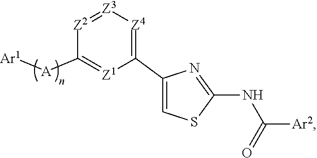

wherein n is selected from 0 and 1; wherein A, when present, is selected from O, NH, N(C1-C4 alkyl), and N(Ar³); wherein Ar³, when present, is phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of Z¹, Z², Z³, and Z⁴ is independently selected from N and CR¹⁰, provided that at least two of Z¹, Z², Z³, and Z⁴ are CR¹⁰; wherein each occurrence of R¹⁰, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², —SO₂R²³, and Cy¹; wherein each occurrence of R²⁰, R²¹, R²², and R²³, when present, is independently selected from hydrogen and C1-C4 alkyl; wherein Cy¹, when present, is selected from 5-membered heterocycle and 6-membered heterocycle, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl; and either: (a) wherein Ar¹ is selected from pyridinyl, piperazinyl, and 6-membered aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴; wherein each occurrence of R¹, R¹², R¹³, and R¹⁴, when present, is independently selected from hydrogen and C1-C4 alkyl; and wherein Ar² is selected from 5-membered heterocycle, 6-membered heterocycle, 5-membered heteroaryl, and 6-membered heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸; and wherein each occurrence of R, R¹⁶, R¹⁷, and R¹⁸, when present, is independently selected from hydrogen and C1-C4 alkyl; or (b) wherein Ar¹ is a structure represented by a formula:

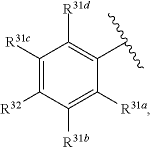

wherein each of R^{31a}, R^{31b}, R^{31c}, and R^{31d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴; wherein R³² is selected from —CN and —CH₂NH₂; and wherein Ar² is selected from 5-membered heterocycle, 6-membered heterocycle, 5-membered heteroaryl, 6-membered heteroaryl, 6-membered aryl, and bicyclo[1.1.1]pentanyl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸, or a pharmaceutically acceptable salt thereof.

In a further aspect, the compound has a structure represented by a formula:

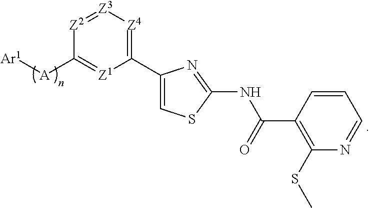

In a further aspect, the compound has a structure represented by a formula:

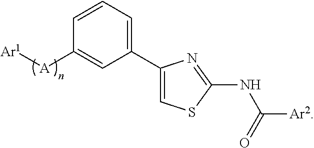

In a further aspect, the compound has a structure represented by a formula:

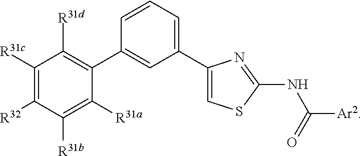

In a further aspect, the compound has a structure represented by a formula:

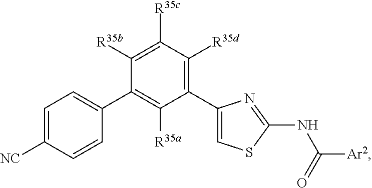

wherein each of R^{35a}, R^{35b}, R^{35c}, and R^{35d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³.

In a further aspect, the compound has a structure represented by a formula:

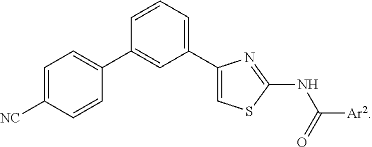

In a further aspect, the compound has a structure represented by a formula:

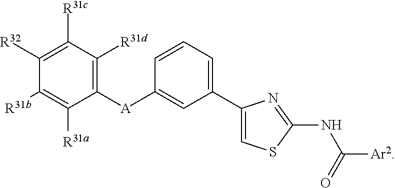

In a still further aspect, A is NH.

In a further aspect, the compound has a structure represented by a formula:

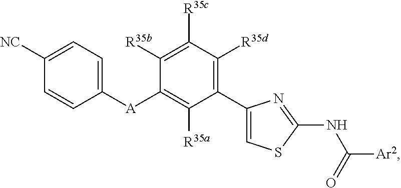

wherein each of R^{35a}, R^{35b}, R^{35c}, and R^{35d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³. In a still further aspect, A is NH.

In a further aspect, the compound is selected from:

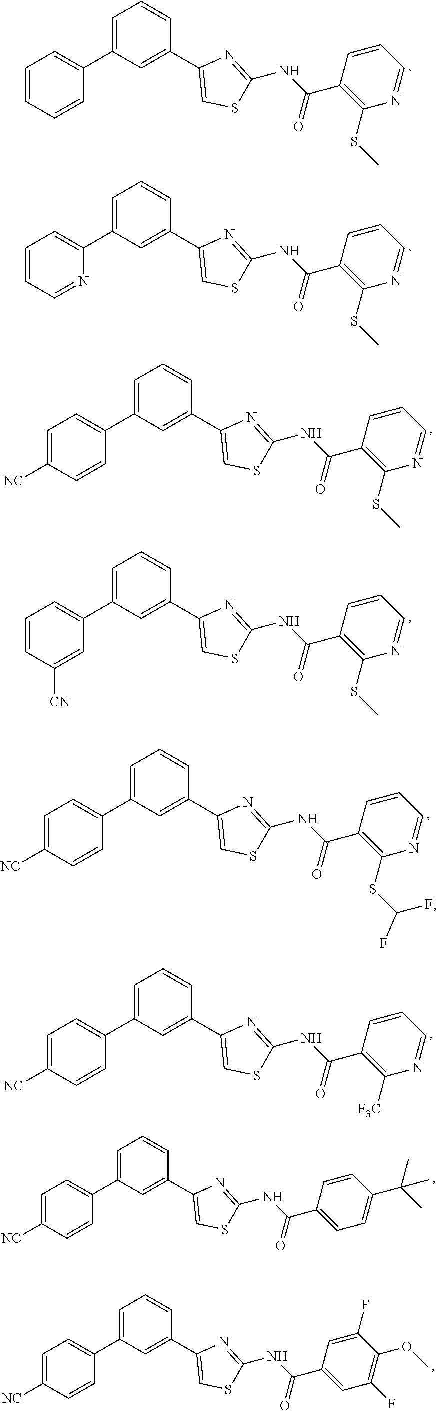

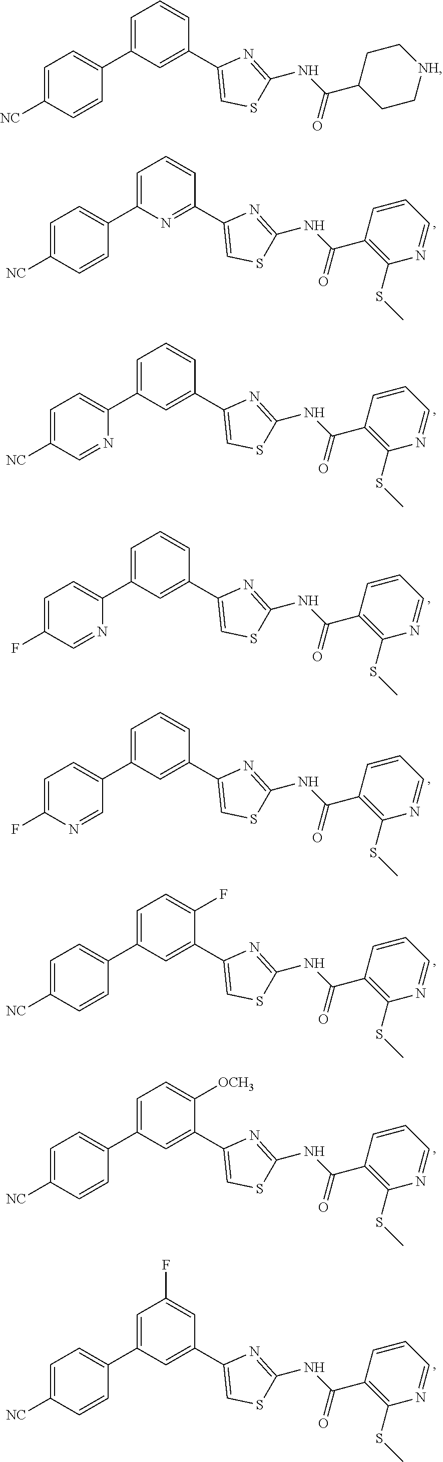

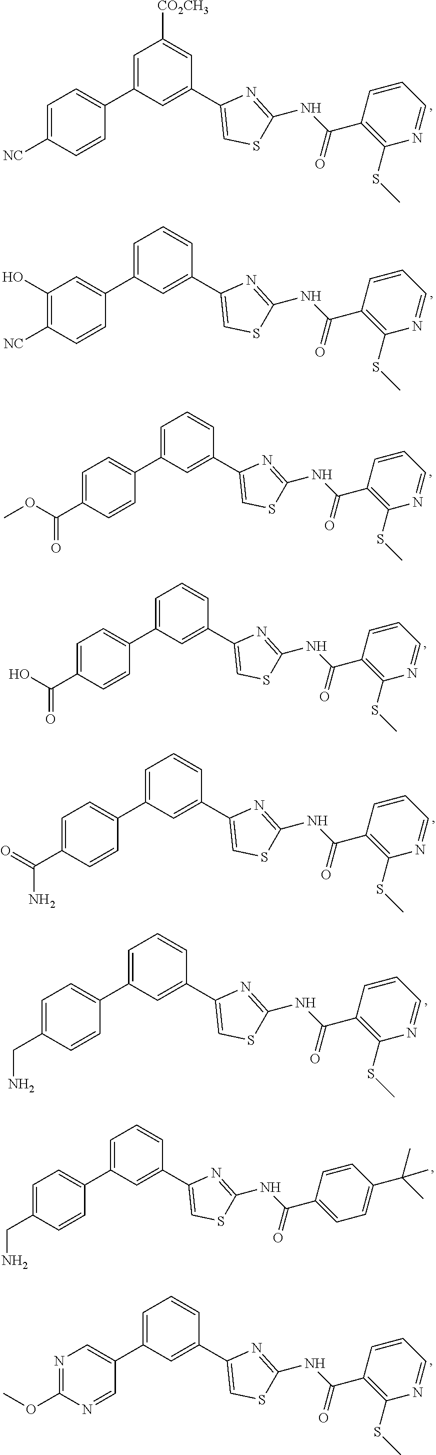

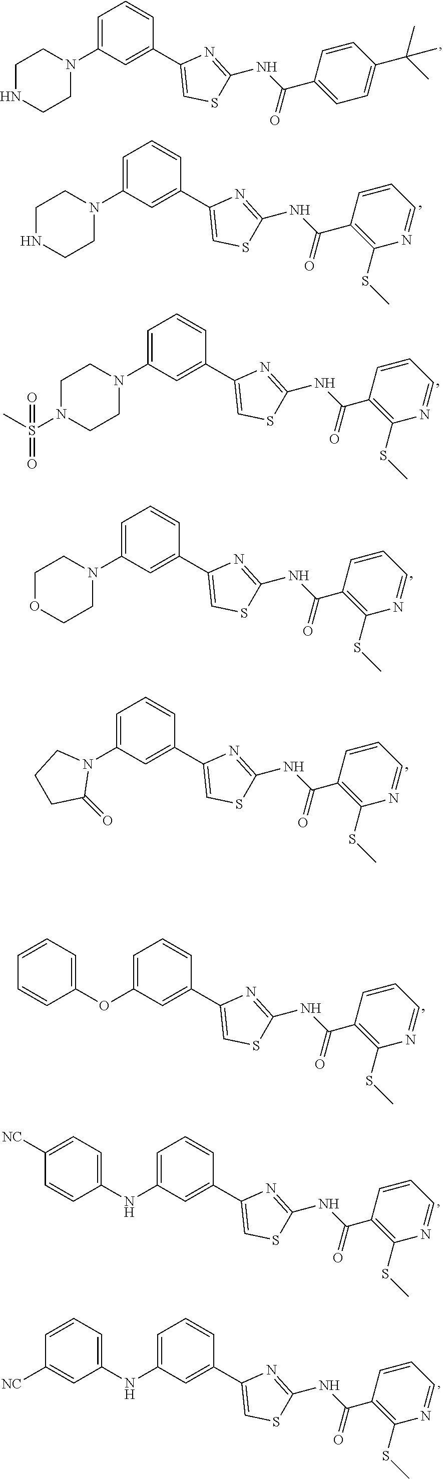

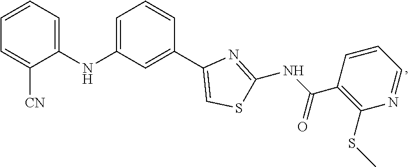 [SMILES: CSc1ncccc1C(=O)Nc1nc(-c2cccc(Nc3ccccc3C#N)c2)cs1]

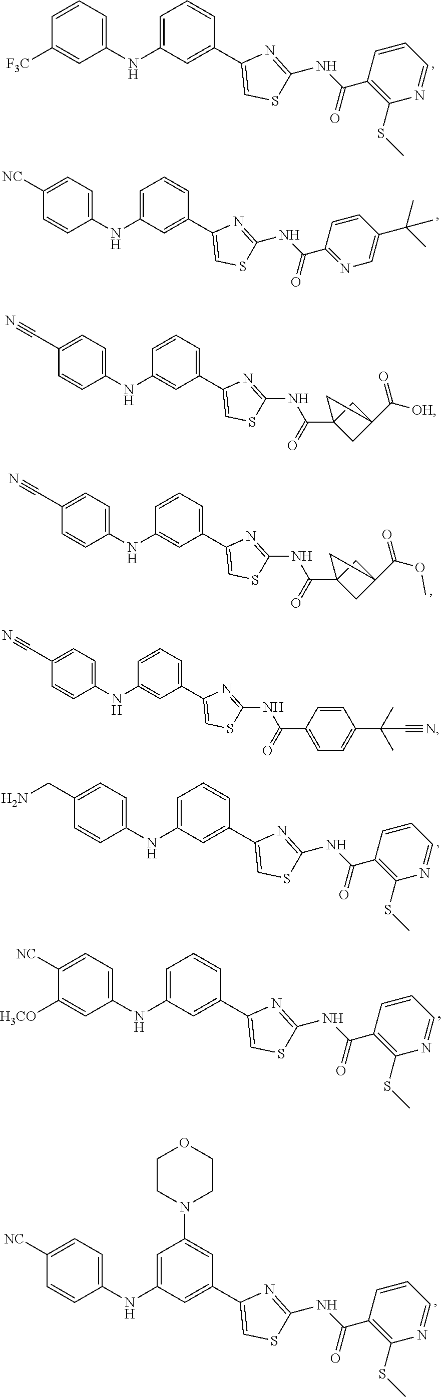

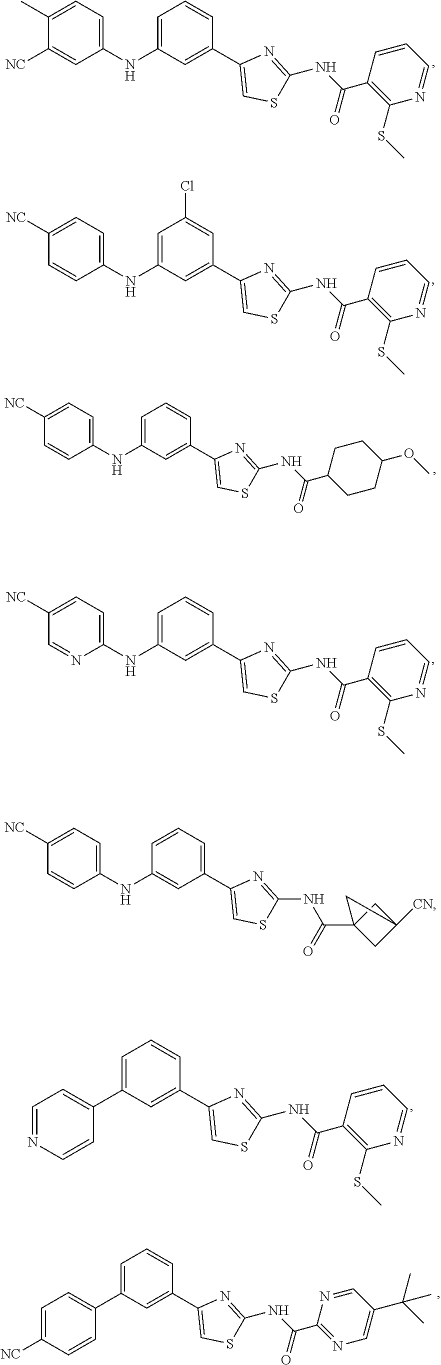

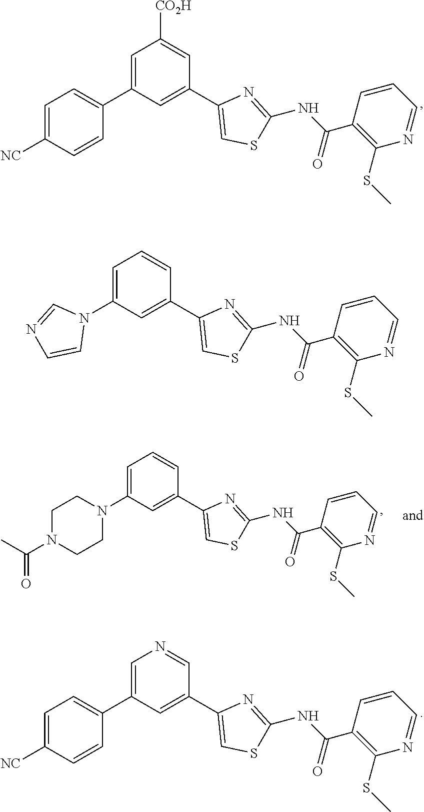 [SMILES: CSc1ncccc1C(=O)Nc1nc(-c2cc(C(=O)O)cc(-c3ccc(C#N)cc3)c2)cs1 ; CSc1ncccc1C(=O)Nc1nc(-c2cccc(-n3ccnc3)c2)cs1 ; CSc1ncccc1C(=O)Nc1nc(-c2cccc(N3CCN(C(C)=O)CC3)c2)cs1 ; CSc1ncccc1C(=O)Nc1nc(-c2cncc(-c3ccc(C#N)cc3)c2)cs1]

In a further aspect, each of R¹ and R² is hydrogen.

In a further aspect, Ar¹ is selected from imidazolyl, pyridinyl, morpholinyl, piperazinyl, and 6-membered aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴; and wherein Ar² is selected from 5-membered heterocycle, 6-membered heterocycle, 5-membered heteroaryl, and 6-membered heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸.

In a further aspect, Ar¹ is a structure represented by a formula:

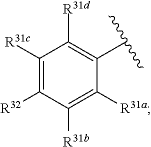

and
wherein Ar² is selected from 5-membered heterocycle, 6-membered heterocycle, 5-membered heteroaryl, 6-membered heteroaryl, 6-membered aryl, and bicyclo[1.1.1]pentanyl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸.

In one aspect, n is selected from 0 and 1. In a further aspect, n is 0. In a still further aspect, n is 1.

a. A Groups

In one aspect, A, when present, is selected from O, NH, N(C1-C4 alkyl), and N(Ar³). In a further aspect, A, when present, is selected from O, NH, N(CH₃), N(CH₂CH₃), N(CH₂CH₂CH₃), N(C(CH₃)₂), and N(Ar³). In a still further aspect, A, when present, is selected from O, NH, N(CH₃), N(CH₂CH₃), and N(Ar³). In yet a further aspect, A, when present, is selected from O, NH, N(CH₃), and N(Ar³).

In various aspects, A, when present, is selected from NH, N(C1-C4 alkyl), and N(Ar³). In a further aspect, A, when present, is selected from NH, N(CH₃), N(CH₂CH₃), N(CH₂CH₂CH₃), and N(C(CH₃)₂). In a still further aspect, A, when present, is selected from NH, N(CH₃), and N(CH₂CH₃). In yet a further aspect, A, when present, is selected from NH and N(CH₃).

In various aspects, A, when present, is N(C1-C4 alkyl). In a further aspect, A, when present, is selected from N(CH₃), N(CH₂CH₃), N(CH₂CH₂CH₃), and N(C(CH₃)₂). In a still further aspect, A, when present, is selected from N(CH₃) and N(CH₂CH₃). In yet a further aspect, A, when present, is N(CH₃).

In a further aspect, A, when present, is N(Ar³).

In a further aspect, A, when present, is selected from O and NH. In a still further aspect, A, when present, is O. In yet a further aspect, A, when present, is NH.

b. Z², Z², Z³, and Z⁴ Groups

In one aspect, each of Z¹, Z², Z³, and Z⁴ is independently selected from N and CR¹⁰, provided that at least two of Z¹, Z², Z³, and Z⁴ are CR¹⁰. In a further aspect, each of Z¹, Z², Z³, and Z⁴ is CR¹⁰. In a further aspect, one of Z¹, Z², Z³, and Z⁴ is O and three of Z¹, Z², Z³, and Z⁴ is CR¹⁰.

In a further aspect, Z¹ and Z² are CR¹⁰ and Z³ and Z⁴ are N. In a still further aspect, Z¹ and Z³ are CR¹⁰ and Z² and Z⁴ are N. In yet a further aspect, Z¹ and Z⁴ are CR⁰ and Z² and Z³ are N. In an even further aspect, Z² and Z³ are CR¹⁰ and Z¹ and Z⁴ are N. In a still further aspect, Z² and Z⁴ are CR¹⁰ and Z¹ and Z³ are N. In yet a further aspect, Z³ and Z⁴ are CR¹⁰ and Z¹ and Z² are N.

In a further aspect, each of Z¹, Z², and Z³ is CR¹⁰ and Z⁴ is N. In a still further aspect, each of Z¹, Z², and Z⁴ is CR¹⁰ and Z³ is N. In yet a further aspect, each of Z¹, Z³, and Z⁴ is CR¹⁰ and Z² is N. In an even further aspect, each of Z², Z³, and Z⁴ is CR¹⁰ and Z¹ is N.

c. R¹⁰ Groups

In one aspect, each occurrence of R¹⁰, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², —SO₂R²³, and Cy¹. In a further aspect, each occurrence of R¹⁰, when present, is independently selected from hydrogen, —F, —Cl, —NH₂, —CN, —OH, —NO₂, methyl, ethyl, n-propyl, i-propyl, ethenyl, propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, —CH(CH₃)CH₂NH₂, —C(O)R²⁰, —CO₂R²¹, —C(O)NR²², —SO₂R²³, and Cy¹. In a still further aspect, each occurrence of R¹⁰, when present, is independently selected from hydrogen, —F, —Cl, —NH₂, —CN, —OH, —NO₂, methyl, ethyl, ethenyl, —CH₂F, —CH₂Cl, —CH₂CH₂F, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —NHCH₃, —NHCH₂CH₃, —N(CH₃)₂, —N(CH₂CH₃)₂, —CH₂NH₂, —CH₂CH₂NH₂, —C(O)R²⁰, —CO₂R²¹, —C(O)NR²², —SO₂R²³, and Cy¹. In yet a further aspect, each occurrence of R¹⁰, when present, is independently selected from hydrogen, —F, —Cl, —NH₂, —CN, —OH, —NO₂, methyl, —CH₂F, —CH₂Cl, —CH₂CN, —CH₂OH, —OCF₃, —OCH₃, —NHCH₃, —N(CH₃)₂, —CH₂NH₂, —C(O)R²⁰, —CO₂R²¹, —C(O)NR²², —SO₂R²³, and Cy¹.

In various aspects, each occurrence of R¹⁰, when present, is independently selected from hydrogen, halogen, —CN, —NH₂, —OH, —NO₂, C1-C4 alkyl, C2-C4 alkenyl, C1-C4 haloalkyl, C1-C4 haloalkoxy, —C(O)R²⁰, —CO₂R²¹, —C(O)NR²², —SO₂R²³, and Cy¹. In a further aspect, each occurrence of R¹⁰, when present, is independently selected from hydrogen, —F, —Cl, —NH₂, —CN, —OH, —NO₂, methyl, ethyl, n-propyl, i-propyl, ethenyl, propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCF₃, —OCH₂CF₃, —OCH₂CH₂CF₃, —OCH(CH₃)CF₃, —C(O)R²⁰, —CO₂R²¹, —C(O)NR²², —SO₂R²³, and Cy¹. In a still further aspect, each occurrence of R¹⁰, when present, is independently selected from hydrogen, —F, —Cl, —NH₂, —CN, —OH, —NO₂, methyl, ethyl, ethenyl, —CH₂F, —CH₂Cl, —CH₂CH₂F, —CH₂CH₂Cl, —OCF₃, —OCH₂CF₃, —OCH₃, —OCH₂CH₃, —C(O)R²⁰, —CO₂R²¹, —C(O)NR²², —SO₂R²³, and Cy¹. In yet a further aspect, each occurrence of R¹⁰, when present, is independently selected from hydrogen, —F, —Cl, —NH₂, —CN, —OH, —NO₂, methyl, —CH₂F, —CH₂Cl, —OCF₃, —C(O)R²⁰, —CO₂R²¹, —C(O)NR²², —SO₂R²³, and Cy¹.

In various aspects, each occurrence of R¹⁰, when present, is independently selected from hydrogen, halogen, C1-C4 haloalkyl, and C1-C4 haloalkoxy. In a further aspect, each occurrence of R¹⁰, when present, is independently selected from hydrogen, —F, —Cl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCF₃, —OCH₂CF₃, —OCH₂CH₂CF₃, and —OCH(CH₃)CF₃. In a still further aspect, each occurrence of R¹⁰, when present, is independently selected from hydrogen, —F, —Cl, —CH₂F, —CH₂Cl, —CH₂CH₂F, —CH₂CH₂Cl, —OCF₃, and —OCH₂CF₃. In yet a further aspect, each occurrence of R¹⁰, when present, is independently selected from hydrogen, —F, —Cl, —CH₂F, —CH₂Cl, and —OCF₃.

In various aspects, each occurrence of R¹⁰, when present, is independently selected from hydrogen, halogen, —CN, —NH₂, —OH, —NO₂, C1-C4 alkyl, C2-C4 alkenyl, C1-C4 cyanoalkyl, C1-C4 hydroxyalkyl, C1-C4 alkoxy, —C(O)R²⁰, —CO₂R²¹, —C(O)NR²², —SO₂R²³, and Cy¹. In a further aspect, each occurrence of R¹⁰, when present, is independently selected from hydrogen, —F, —Cl, —NH₂, —CN, —OH, —NO₂, methyl, ethyl, n-propyl, i-propyl, ethenyl, propenyl, isopropenyl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)CH₃, —C(O)R²⁰, —CO₂R²¹, —C(O)NR²², —SO₂R²³, and Cy¹. In a still further aspect, each occurrence of R¹⁰, when present, is independently selected from hydrogen, —F, —Cl, —NH₂, —CN, —OH, —NO₂, methyl, ethyl, ethenyl, —CH₂CN, —CH₂CH₂CN, —CH₂OH, —CH₂CH₂OH, —OCH₃, —OCH₂CH₃, —C(O)R²⁰, —CO₂R²¹, —C(O)NR²², —SO₂R²³, and Cy¹. In yet a further aspect, each occurrence of R¹⁰, when present, is independently selected from hydrogen, —F, —Cl, —NH₂, —CN, —OH, —NO₂, methyl, —CH₂CN, —CH₂OH, —OCH₃, —C(O)R²⁰, —CO₂R²¹, —C(O)NR²², —SO₂R²³, and Cy¹.

In various aspects, each occurrence of R¹⁰, when present, is independently selected from hydrogen, halogen, C1-C4 cyanoalkyl, C1-C4 hydroxyalkyl, and C1-C4 alkoxy. In a further aspect, each occurrence of R¹⁰, when present, is independently selected from hydrogen, —F, —Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₃, —OCH₂CH₃, —OCH₂CH₂CH₃, and —OCH(CH₃)CH₃. In a still further aspect, each occurrence of R¹⁰, when present, is independently selected from hydrogen, —F, —Cl, —CH₂CN, —CH₂CH₂CN, —CH₂OH, —CH₂CH₂OH, —OCH₃, and —OCH₂CH₃. In yet a further aspect, each occurrence of R¹⁰, when present, is independently selected from hydrogen, —F, —Cl, —CH₂CN, —CH₂OH, and —OCH₃.

In various aspects, each occurrence of R¹⁰, when present, is independently selected from hydrogen, halogen, —CN, —NH₂, —OH, —NO₂, C1-C4 alkyl, C2-C4 alkenyl, C1-C4 alkylamino, (C1-C4)(C1-C4) dialkylamino, C1-C4 aminoalkyl, —C(O)R²⁰, —CO₂R²¹, —C(O)NR²², —SO₂R²³, and Cy¹. In a further aspect, each occurrence of R¹⁰, when present, is independently selected from hydrogen, —F, —Cl, —NH₂, —CN, —OH, —NO₂, methyl, ethyl, n-propyl, i-propyl, ethenyl, propenyl, isopropenyl, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, —CH(CH₃)CH₂NH₂, —C(O)R²⁰, —CO₂R²¹, —C(O)NR²², —SO₂R²³, and Cy¹. In a still further aspect, each occurrence of R¹⁰, when present, is independently selected from hydrogen, —F, —Cl, —NH₂, —CN, —OH, —NO₂, methyl, ethyl, ethenyl, —NHCH₃, —NHCH₂CH₃, —N(CH₃)₂, —N(CH₂CH₃)₂, —CH₂NH₂, —CH₂CH₂NH₂, —C(O)R²⁰, —CO₂R²¹, —C(O)NR²², —SO₂R²³, and Cy¹. In yet a further aspect, each occurrence of R¹⁰, when present, is independently selected from hydrogen, —F, —Cl, —NH₂, —CN, —OH, —NO₂, methyl, —NHCH₃, —N(CH₃)₂, —CH₂NH₂, —C(O)R²⁰, —CO₂R²¹, —C(O)NR²², —SO₂R²³, and Cy¹.

In various aspects, each occurrence of R¹⁰, when present, is independently selected from hydrogen, halogen, C1-C4 alkylamino, (C1-C4)(C1-C4) dialkylamino, and C1-C4 aminoalkyl. In a further aspect, each occurrence of R¹⁰, when present, is independently selected from hydrogen, —F, —Cl, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a still further aspect, each occurrence of R¹⁰, when present, is independently selected from hydrogen, —F, —Cl, —NHCH₃, —NHCH₂CH₃, —N(CH₃)₂, —N(CH₂CH₃)₂, —CH₂NH₂, and —CH₂CH₂NH₂. In yet a further aspect, each occurrence of R¹⁰, when present, is independently selected from hydrogen, —F, —Cl, —NHCH₃, —N(CH₃)₂, and —CH₂NH₂.

In various aspects, each occurrence of R¹⁰, when present, is independently selected from hydrogen, halogen, —CN, —NH₂, —OH, —NO₂, C1-C4 alkyl, C2-C4 alkenyl, —C(O)R²⁰, —CO₂R²¹, —C(O)NR²², —SO₂R²³, and Cy¹. In a further aspect, each occurrence of R¹⁰, when present, is independently selected from hydrogen, —F, —Cl, —NH₂, —CN, —OH, —NO₂, methyl, ethyl, n-propyl, i-propyl, ethenyl, propenyl, isopropenyl, —C(O)R²⁰, —CO₂R²¹, —C(O)NR²², —SO₂R²³, and Cy¹. In a still further aspect, each occurrence of R¹⁰, when present, is independently selected from hydrogen, —F, —Cl, —NH₂, —CN, —OH, —NO₂, methyl, ethyl, ethenyl, —C(O)R²⁰, —CO₂R²¹, —C(O)NR²², —SO₂R²³, and Cy¹. In yet a further aspect, each occurrence of R¹⁰, when present, is independently selected from hydrogen, —F, —Cl, —NH₂, —CN, —OH, —NO₂, methyl, —C(O)R²⁰, —CO₂R²¹, —C(O)NR²², —SO₂R²³, and Cy¹.

In various aspects, each occurrence of R¹⁰, when present, is independently selected from hydrogen, halogen, C1-C4 alkyl, and C2-C4 alkenyl. In a further aspect, each occurrence of R¹⁰, when present, is independently selected from hydrogen, —F, —Cl, methyl, ethyl, n-propyl, i-propyl, ethenyl, propenyl, and isopropenyl. In a still further aspect, each occurrence of R¹⁰, when present, is independently selected from hydrogen, —F, —Cl, methyl, ethyl, and ethenyl. In yet a further aspect, each occurrence of R¹⁰, when present, is independently selected from hydrogen, —F, —Cl, and methyl.

In a further aspect, each occurrence of R¹⁰, when present, is independently selected from hydrogen, halogen, —CN, —NH₂, —OH, —NO₂, —C(O)R²⁰, —CO₂R²¹, —C(O)NR²², —SO₂R²³, and Cy¹.

In a further aspect, each occurrence of R¹⁰, when present, is independently selected from hydrogen, halogen, C₁-C₄ alkoxy, and —CO₂R²¹. In a still further aspect, each occurrence of R¹⁰, when present, is independently selected from hydrogen, —F, —Cl, —OCH₃, —OCH₂CH₃, —OCH₂CH₂CH₃, —OCH(CH₃)CH₃, and —CO₂R²¹. In yet a further aspect, each occurrence of R¹⁰, when present, is independently selected from hydrogen, —F, —Cl, —OCH₃, —OCH₂CH₃, and —CO₂R²¹. In an even further aspect, each occurrence of R¹⁰, when present, is independently selected from hydrogen, —F, —Cl, —OCH₃, and —CO₂R²¹.

In a further aspect, each occurrence of R¹⁰, when present, is hydrogen.

d. R¹¹, R¹², R¹³, and R¹⁴ Groups

In one aspect, each occurrence of R¹, R¹², R¹³, and R¹⁴, when present, is independently selected from hydrogen and C1-C4 alkyl. In a further aspect, each occurrence of R¹, R, R¹³, and R¹⁴, when present, is independently selected from hydrogen, methyl, ethyl, n-propyl, and isopropyl. In a still further aspect, each occurrence of R¹¹, R¹², R¹³, and R¹⁴, when present, is independently selected from hydrogen, methyl, and ethyl. In yet a further aspect, each occurrence of R¹¹, R¹², R¹³, and R¹⁴, when present, is independently selected from hydrogen and ethyl. In a still further aspect, each occurrence of R¹¹, R¹², R¹³, and R¹⁴, when present, is independently selected from hydrogen and methyl.

In various aspects, each occurrence of R¹¹, R¹², R¹³, and R¹⁴, when present, is C1-C4 alkyl. In a further aspect, each occurrence of R¹¹, R¹², R¹³, and R¹⁴, when present, is independently selected from methyl, ethyl, n-propyl, and isopropyl. In a still further aspect, each occurrence of R¹¹, R¹², R¹³, and R¹⁴, when present, is independently selected from methyl and ethyl. In yet a further aspect, each occurrence of R¹¹, R¹², R¹³, and R¹⁴, when present, is ethyl. In a still further aspect, each occurrence of R¹¹, R¹², R¹³, and R¹⁴, when present, is methyl.

In a further aspect, each occurrence of R¹¹, R¹², R¹³, and R¹⁴, when present, is hydrogen.

e. R¹⁵, R¹⁶, R¹⁷, and R¹⁸ Groups

In one aspect, each occurrence of R¹⁵, R¹⁶, R¹⁷, and R¹⁸, when present, is independently selected from hydrogen and C1-C4 alkyl. In a further aspect, each occurrence of R¹⁵, R¹⁶, R¹⁷, and R¹⁸, when present, is independently selected from hydrogen, methyl, ethyl, n-propyl, and isopropyl. In a still further aspect, each occurrence of R¹⁵, R¹⁶, R¹⁷, and R¹⁸, when present, is independently selected from hydrogen, methyl, and ethyl. In yet a further aspect, each occurrence of R¹⁵, R¹⁶, R¹⁷, and R¹⁸, when present, is independently selected from hydrogen and ethyl. In a still further aspect, each occurrence of R¹⁵, R¹⁶, R¹⁷, and R¹⁸, when present, is independently selected from hydrogen and methyl.

In various aspects, each occurrence of R¹⁵, R¹⁶, R¹⁷, and R¹⁸, when present, is C1-C4 alkyl. In a further aspect, each occurrence of R¹⁵, R¹⁶, R¹⁷, and R¹⁸, when present, is independently selected from methyl, ethyl, n-propyl, and isopropyl. In a still further aspect, each occurrence of R¹⁵, R¹⁶, R¹⁷, and R¹⁸, when present, is independently selected from methyl and ethyl. In yet a further aspect, each occurrence of R¹⁵, R¹⁶, R¹⁷, and R¹⁸, when present, is ethyl. In a still further aspect, each occurrence of R¹⁵, R¹⁶, R¹⁷, and R¹⁸, when present, is methyl.

In a further aspect, each occurrence of R¹⁵, R¹⁶, R¹⁷, and R¹⁸, when present, is hydrogen.

f. R²⁰, R²¹, R²², and R²³ Groups

In one aspect, each occurrence of R²⁰, R²¹, R²², and R²³, when present, is independently selected from hydrogen and C1-C4 alkyl. In a further aspect, each occurrence of R²⁰, R²¹, R²², and R²³, when present, is independently selected from hydrogen, methyl, ethyl, n-propyl, and isopropyl. In a still further aspect, each occurrence of R²⁰, R²¹, R²², and R²³, when present, is independently selected from hydrogen, methyl, and ethyl. In yet a further aspect, each occurrence of R²⁰, R²¹, R²², and R²³, when present, is independently selected from hydrogen and ethyl. In a still further aspect, each occurrence of R²⁰, R²¹, R²², and R²³, when present, is independently selected from hydrogen and methyl.

In various aspects, each occurrence of R²⁰, R²¹, R²², and R²³, when present, is C1-C4 alkyl. In a further aspect, each occurrence of R²⁰, R²¹, R²², and R²³, when present, is independently selected from methyl, ethyl, n-propyl, and isopropyl. In a still further aspect, each occurrence of R²⁰, R²¹, R²², and R²³, when present, is independently selected from methyl and ethyl. In yet a further aspect, each occurrence of R²⁰, R²¹, R²², and R²³, when present, is ethyl. In a still further aspect, each occurrence of R²⁰, R²¹, R²², and R²³, when present, is methyl.

In a further aspect, each occurrence of R²⁰, R²¹, R²², and R²³, when present, is hydrogen.

G. R^{31A}, R^{31B}, R^{31C}, AND R^{31D} Groups

In one aspect, each of R^{31a}, R^{31b}, R^{31c}, and R^{31d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴. In a further aspect, each of R^{31a}, R^{31b}, R^{31c}, and R^{31d} is independently selected from hydrogen, —F, —Cl, —NH₂, —CN, —OH, —NO₂, methyl, ethyl, n-propyl, i-propyl, ethenyl, propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, —CH(CH₃)CH₂NH₂, —C(O)R¹¹, —CO₂R¹², —C(O)NR¹³, and —SO₂R¹⁴. In a still further aspect, each of R^{31a}, R^{31b}, R³¹, and R^{31d} is independently selected from hydrogen, —F, —Cl, —NH₂, —CN, —OH, —NO₂, methyl, ethyl, ethenyl, —CH₂F, —CH₂Cl, —CH₂CH₂F, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —NHCH₃, —NHCH₂CH₃, —N(CH₃)₂, —N(CH₂CH₃)₂, —CH₂NH₂, —CH₂CH₂NH₂, —C(O)R¹¹, —CO₂R¹², —C(O)NR¹³, and —SO₂R¹⁴. In yet a further aspect, each of R^{31a}, R^{31b}, R^{31c}, and R^{31d} is independently selected from hydrogen, —F, —Cl, —NH₂, —CN, —OH, —NO₂, methyl, —CH₂F, —CH₂Cl, —CH₂CN, —CH₂OH, —OCF₃, —OCH₃, —NHCH₃, —N(CH₃)₂, —CH₂NH₂, —C(O)R¹¹, —CO₂R¹², —C(O)NR¹³, and —SO₂R¹⁴.

In various aspects, each of R^{31a}, R^{31b}, R^{31c}, and R^{31d} is independently selected from hydrogen, halogen, —CN, —NH₂, —OH, —NO₂, C1-C4 alkyl, C2-C4 alkenyl, C1-C4 haloalkyl, C1-C4 haloalkoxy, —C(O)R¹¹, —CO₂R¹², —C(O)NR¹³, and —SO₂R¹⁴. In a further aspect, each of R^{31a}, R^{31b}, R^{31c}, and R^{31d} is independently selected from hydrogen, —F, —Cl, —NH₂, —CN, —OH, —NO₂, methyl, ethyl, n-propyl, i-propyl, ethenyl, propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCF₃, —OCH₂CF₃, —OCH₂CH₂CF₃, —OCH(CH₃)CF₃, —C(O)R¹¹, —CO₂R¹², —C(O)NR¹³, and —SO₂R¹⁴. In a still further aspect, each of R^{31a}, R^{31b}, R^{31c}, and R^{31d} is independently selected from hydrogen, —F, —Cl, —NH₂, —CN, —OH, —NO₂, methyl, ethyl, ethenyl, —CH₂F, —CH₂Cl, —CH₂CH₂F, —CH₂CH₂Cl, —OCF₃, —OCH₂CF₃, —OCH₃, —OCH₂CH₃, —C(O)R¹¹, —CO₂R¹², —C(O)NR¹³, and —SO₂R¹⁴. In yet a further aspect, each of R^{31a}, R^{31b}, R^{31c}, and R^{31d} is independently selected from hydrogen, —F, —Cl, —NH₂, —CN, —OH, —NO₂, methyl, —CH₂F, —CH₂Cl, —OCF₃, —C(O)R¹¹, —CO₂R¹², —C(O)NR¹³, and —SO₂R¹⁴.

In various aspects, each of R^{31a}, R^{31b}, R^{31c}, and R^{31d} is independently selected from hydrogen, halogen, C1-C4 haloalkyl, and C1-C4 haloalkoxy. In a further aspect, each occurrence of R¹⁰, when present, is independently selected from hydrogen, —F, —Cl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCF₃, —OCH₂CF₃, —OCH₂CH₂CF₃, and —OCH(CH₃)CF₃. In a still further aspect, each of R^{31a}, R^{31b}, R^{31c}, and R^{31d} is independently selected from hydrogen, —F, —Cl, —CH₂F, —CH₂Cl, —CH₂CH₂F, —CH₂CH₂Cl, —OCF₃, and —OCH₂CF₃. In yet a further aspect, each of R^{31a}, R^{31b}, R^{31c}, and R^{31d} is independently selected from hydrogen, —F, —Cl, —CH₂F, —CH₂Cl, and —OCF₃.

In various aspects, each of R^{31a}, R^{31b}, R^{31c}, and R^{31d} is independently selected from hydrogen, halogen, —CN, —NH₂, —OH, —NO₂, C1-C4 alkyl, C2-C4 alkenyl, C1-C4 cyanoalkyl, C1-C4 hydroxyalkyl, C1-C4 alkoxy, —C(O)R¹¹, —CO₂R¹², —C(O)NR¹³, and —SO₂R¹⁴. In a further aspect, each of R^{31a}, R^{31b}, R^{31c}, and R^{31d} is independently selected from hydrogen, —F, —Cl, —NH₂, —CN, —OH, —NO₂, methyl, ethyl, n-propyl, i-propyl, ethenyl, propenyl, isopropenyl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)CH₃, —C(O)R¹¹, —CO₂R¹², —C(O)NR¹³, and —SO₂R¹⁴. In a still further aspect, each of R^{31a}, R^{31b}, R^{31c}, and R^{31d} is independently selected from hydrogen, —F, —Cl, —NH₂, —CN, —OH, —NO₂, methyl, ethyl, ethenyl, —CH₂CN, —CH₂CH₂CN, —CH₂OH, —CH₂CH₂OH, —OCH₃, —OCH₂CH₃, —C(O)R¹¹, —CO₂R¹², —C(O)NR¹³, and —SO₂R¹⁴. In yet a further aspect, each of R^{31a}, R^{31b}, R^{31c}, and R^{31d} is independently selected from hydrogen, —F, —Cl, —NH₂, —CN, —OH, —NO₂, methyl, —CH₂CN, —CH₂OH, —OCH₃, —C(O)R¹¹, —CO₂R¹², —C(O)NR¹³, and —SO₂R¹⁴.

In various aspects, each of R^{31a}, R^{31b}, R^{31c}, and R^{31d} is independently selected from hydrogen, halogen, C1-C4 cyanoalkyl, C1-C4 hydroxyalkyl, and C1-C4 alkoxy. In a further aspect, each of R^{31a}, R^{31b}, R^{31c}, and R^{31d} is independently selected from hydrogen, —F, —Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₃, —OCH₂CH₃, —OCH₂CH₂CH₃, and —OCH(CH₃)CH₃. In a still further aspect, each of R^{31a}, R^{31b}, R^{31c}, and R^{31d} is independently selected from hydrogen, —F, —Cl, —CH₂CN, —CH₂CH₂CN, —CH₂OH, —CH₂CH₂OH, —OCH₃, and —OCH₂CH₃. In yet a further aspect, each of R^{31a}, R^{31b}, R^{31c}, and R^{31d} is independently selected from hydrogen, —F, —Cl, —CH₂CN, —CH₂OH, and —OCH₃.

In various aspects, each of R^{31a}, R^{31b}, R^{31c}, and R^{31d} is independently selected from hydrogen, halogen, —CN, —NH₂, —OH, —NO₂, C1-C4 alkyl, C2-C4 alkenyl, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴. In a further aspect, each of R^{31a}, R^{31b}, R^{31c}, and R^{31d} is independently selected from hydrogen, —F, —Cl, —NH₂, —CN, —OH, —NO₂, methyl, ethyl, n-propyl, i-propyl, ethenyl, propenyl, isopropenyl, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, —CH(CH₃)CH₂NH₂, —C(O)R¹¹, —CO₂R¹², —C(O)NR¹³, and —SO₂R¹⁴. In a still further aspect, each of R^{31a}, R^{31b}, R³¹, and R^{31d} is independently selected from hydrogen, —F, —Cl, —NH₂, —CN, —OH, —NO₂, methyl, ethyl, ethenyl, —NHCH₃, —NHCH₂CH₃, —N(CH₃)₂, —N(CH₂CH₃)₂, —CH₂NH₂, —CH₂CH₂NH₂, —C(O)R¹¹, —CO₂R¹², —C(O)NR¹³, and —S₂R¹⁴. In yet a further aspect, each of R^{31a}, R^{31b}, R^{31c}, and R^{31d} is independently selected from hydrogen, —F, —Cl, —NH₂, —CN, —OH, —NO₂, methyl, —NHCH₃, —N(CH₃)₂, —CH₂NH₂, —C(O)R¹¹, —CO₂R¹², —C(O)NR¹³, and —SO₂R¹⁴.

In various aspects, each of R^{31a}, R^{31b}, R^{31c}, and R^{31d} is independently selected from hydrogen, halogen, C1-C4 alkylamino, (C1-C4)(C1-C4) dialkylamino, and C1-C4 aminoalkyl. In a further aspect, each of R^{31a}, R^{31b}, R^{31c}, and R^{31d} is independently selected from hydrogen, —F, —Cl, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a still further aspect, each of R^{31a}, R^{31b}, R^{31c}, and R^{31d} is independently selected from hydrogen, —F, —Cl, —NHCH₃, —NHCH₂CH₃, —N(CH₃)₂, —N(CH₂CH₃)₂, —CH₂NH₂, and —CH₂CH₂NH₂. In yet a further aspect, each of R^{31a}, R^{31b}, R^{31c}, and R^{31d} is independently selected from hydrogen, —F, —Cl, —NHCH₃, —N(CH₃)₂, and —CH₂NH₂.

In various aspects, each of R^{31a}, R^{31b}, R^{31c}, and R^{31d} is independently selected from hydrogen, halogen, —CN, —NH₂, —OH, —NO₂, C1-C4 alkyl, C2-C4 alkenyl, —C(O)R¹¹, —CO₂R¹², —C(O)NR¹³, and —SO₂R¹⁴. In a further aspect, each of R^{31a}, R^{31b}, R^{31c}, and R^{31d} is independently selected from hydrogen, —F, —Cl, —NH₂, —CN, —OH, —NO₂, methyl, ethyl, n-propyl, i-propyl, ethenyl, propenyl, isopropenyl, —C(O)R¹¹, —CO₂R¹², —C(O)NR¹³, and —SO₂R¹⁴. In a still further aspect, each of R^{31a}, R^{31b}, R^{31c}, and R^{31d} is independently selected from hydrogen, —F, —Cl, —NH₂, —CN, —OH, —NO₂, methyl, ethyl, ethenyl, —C(O)R¹¹, —CO₂R¹², —C(O)NR¹³, and —SO₂R¹⁴. In yet a further aspect, each of R^{31a}, R^{31b}, R^{31c}, and R^{31d} is independently selected from hydrogen, —F, —Cl, —NH₂, —CN, —OH, —NO₂, methyl, —C(O)R¹¹, —CO₂R¹², —C(O)NR¹³, and —SO₂R¹⁴.

In various aspects, each of R^{31a}, R^{31b}, R^{31c}, and R^{31d} is independently selected from hydrogen, halogen, C1-C4 alkyl, and C2-C4 alkenyl. In a further aspect, each of R^{31a}, R^{31b}, R^{31c}, and R^{31d} is independently selected from hydrogen, —F, —Cl, methyl, ethyl, n-propyl, i-propyl, ethenyl, propenyl, and isopropenyl. In a still further aspect, each of R^{31a}, R^{31b}, R³1, and R^{31d} is independently selected from hydrogen, —F, —Cl, methyl, ethyl, and ethenyl. In yet a further aspect, each of R^{31a}, R^{31b}, R^{31c}, and R^{31d} is independently selected from hydrogen, —F, —Cl, and methyl.

In a further aspect, each of R^{31a}, R^{31b}, R^{31c}, and R^{31d} is independently selected from hydrogen, halogen, —CN, —NH₂, —OH, —NO₂, —C(O)R¹¹, —CO₂R¹², —C(O)NR¹³, and —SO₂R¹⁴.

In a further aspect, each of R^{31a}, R^{31b}, R^{31c}, and R^{31d} is hydrogen.

h. R³² Groups

In one aspect, R³² is selected from —CN and —CH₂NH₂. In a further aspect, R³² is —CN. In a still further aspect, R³² is —CH₂NH₂.

i. R^{33A}, R^{33B}, and R^{33C} Groups

In one aspect, each of R^{33a}, R^{33b}, and R^{33c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In a further aspect, each of R^{33a}, R^{33b}, and R³³ is independently selected from hydrogen, —F, —Cl, —NH₂, —CN, —OH, —NO₂, methyl, ethyl, n-propyl, i-propyl, ethenyl, propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —SCF₃, —SCH₂CF₃, —SCH₂CH₂CF₃, —SCH(CH₃)CF₃, —SCH₃, —SCH₂CH₃, —SCH₂CH₂CH₃, —SCH(CH₃)CH₃, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, —CH(CH₃)CH₂NH₂, —C(O)R¹⁵, —CO₂R¹⁶, —C(O)NR¹⁷, and —SO₂R¹⁸. In a still further aspect, each of R^{33a}, R^{33b}, and R³³ is independently selected from hydrogen, —F, —Cl, —NH₂, —CN, —OH, —NO₂, methyl, ethyl, ethenyl, —CH₂F, —CH₂Cl, —CH₂CH₂F, —CH₂CH₂Cl, —CH₂CN, —CH₂CH₂CN, —CH₂OH, —CH₂CH₂OH, —SCF₃, —SCH₂CF₃, —SCH₃, —SCH₂CH₃, —OCF₃, —OCH₂CF₃, —OCH₃, —OCH₂CH₃, —NHCH₃, —NHCH₂CH₃, —N(CH₃)₂, —N(CH₂CH₃)₂, —CH₂NH₂, —CH₂CH₂NH₂, —C(O)R¹⁵, —CO₂R¹⁶, —C(O)NR¹⁷, and —SO₂R¹⁸. In yet a further aspect, each of R^{33a}, R^{33b}, and R^{33c} is independently selected from hydrogen, —F, —Cl, —NH₂, —CN, —OH, —NO₂, methyl, —CH₂F, —CH₂Cl, —CH₂CN, —CH₂OH, —SCF₃, —SCH₃, —OCF₃, —OCH₃, —NHCH₃, —N(CH₃)₂, —CH₂NH₂, —C(O)R¹⁵, —CO₂R¹⁶, —C(O)NR¹⁷, and —SO₂R¹⁸.

In various aspects, each of R^{33a}, R^{33b}, and R^{33c} is independently selected from hydrogen, halogen, —CN, —NH₂, —OH, —NO₂, C1-C4 thioalkyl, C1-C4 thiohaloalkyl, —C(O)R¹⁵, —CO₂R¹⁶, —C(O)NR¹⁷, and —SO₂R¹⁸. In a further aspect, each of R^{33a}, R^{33b}, and R³³ is independently selected from hydrogen, —F, —Cl, —NH₂, —CN, —OH, —NO₂, —SCF₃, —SCH₂CF₃, —SCH₂CH₂CF₃, —SCH(CH₃)CF₃, —SCH₃, —SCH₂CH₃, —SCH₂CH₂CH₃, —SCH(CH₃)CH₃, —C(O)R¹⁵, —CO₂R¹⁶, —C(O)NR¹⁷, and —S₂R¹⁸. In a still further aspect, each of R^{33a}, R^{33b}, and R³³ is independently selected from hydrogen, —F, —Cl, —NH₂, —CN, —OH, —NO₂, —SCF₃, —SCH₂CF₃, —SCH₃, —SCH₂CH₃, —C(O)R¹⁵, —CO₂R¹⁶, —C(O)NR¹⁷, and —SO₂R¹⁸. In yet a further aspect, each of R^{33a}, R^{33b}, and R^{33c} is independently selected from hydrogen, —F, —Cl, —NH₂, —CN, —OH, —NO₂, —SCF₃, —SCH₃, —C(O)R¹⁵, —CO₂R¹⁶, —C(O)NR¹⁷, and —SO₂R¹⁸.

In various aspects, each of R^{33a}, R^{33b}, and R^{33c} is independently selected from hydrogen, halogen, C1-C4 thioalkyl, and C1-C4 thiohaloalkyl. In a further aspect, each of R^{33a}, R^{33b}, and R³³ is independently selected from hydrogen, —F, —Cl, —SCF₃, —SCH₂CF₃, —SCH₂CH₂CF₃, —SCH(CH₃)CF₃, —SCH₃, —SCH₂CH₃, —SCH₂CH₂CH₃, and —SCH(CH₃)CH₃. In a still further aspect, each of R^{33a}, R^{33b}, and R^{33c} is independently selected from hydrogen, —F, —Cl, —SCF₃, —SCH₂CF₃, —SCH₃, and —SCH₂CH₃. In yet a further aspect, each of R^{33a}, R^{33b}, and R³³ is independently selected from hydrogen, —F, —Cl, —SCF₃, and —SCH₃.

In various aspects, each of R^{33a}, R^{33b}, and R^{33c} is independently selected from hydrogen, halogen, —CN, —NH₂, —OH, —NO₂, C1-C4 alkyl, C2-C4 alkenyl, C1-C4 haloalkyl, C1-C4 haloalkoxy, —C(O)R¹⁵, —CO₂R¹⁶, —C(O)NR¹⁷, and —SO₂R¹⁸. In a further aspect, each of R^{33a}, R^{33b}, and R³³ is independently selected from hydrogen, —F, —Cl, —NH₂, —CN, —OH, —NO₂, methyl, ethyl, n-propyl, i-propyl, ethenyl, propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂C, —OCF₃, —OCH₂CF₃, —OCH₂CH₂CF₃, —OCH(CH₃)CF₃, —C(O)R¹⁵, —CO₂R¹⁶, —C(O)NR¹⁷, and —SO₂R¹⁸. In a still further aspect, each of R^{33a}, R^{33b}, and R^{33c} is independently selected from hydrogen, —F, —Cl, —NH₂, —CN, —OH, —NO₂, methyl, ethyl, ethenyl, —CH₂F, —CH₂Cl, —CH₂CH₂F, —CH₂CH₂Cl, —OCF₃, —OCH₂CF₃, —OCH₃, —OCH₂CH₃, —C(O)R¹⁵, —CO₂R¹⁶, —C(O)NR¹⁷, and —SO₂R¹⁸. In yet a further aspect, each of R^{33a}, R^{33b}, and R³³ is independently selected from hydrogen, —F, —Cl, —NH₂, —CN, —OH, —NO₂, methyl, —CH₂F, —CH₂Cl, —OCF₃, —C(O)R¹⁵, —CO₂R¹⁶, —C(O)NR¹⁷, and —SO₂R¹⁸.

In various aspects, each of R^{33a}, R^{33b}, and R^{33c} is independently selected from hydrogen, halogen, C1-C4 haloalkyl, and C1-C4 haloalkoxy. In a further aspect, each of R^{33a}, R^{33b}, and R³³ is independently selected from hydrogen, —F, —Cl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCF₃, —OCH₂CF₃, —OCH₂CH₂CF₃, and —OCH(CH₃)CF₃. In a still further aspect, each of R^{33a}, R^{33b}, and R³³ is independently selected from hydrogen, —F, —Cl, —CH₂F, —CH₂Cl, —CH₂CH₂F, —CH₂CH₂Cl, —OCF₃, and —OCH₂CF₃. In yet a further aspect, each of R^{33a}, R^{33b}, and R³³ is independently selected from hydrogen, —F, —Cl, —CH₂F, —CH₂Cl, and —OCF₃.

In various aspects, each of R^{33a}, R^{33b}, and R^{33c} is independently selected from hydrogen, halogen, —CN, —NH₂, —OH, —NO₂, C1-C4 alkyl, C2-C4 alkenyl, C1-C4 cyanoalkyl, C1-C4 hydroxyalkyl, C1-C4 alkoxy, —C(O)R¹⁵, —CO₂R¹⁶, —C(O)NR¹⁷, and —SO₂R¹⁸. In a further aspect, each of R^{33a}, R^{33b}, and R^{33c} is independently selected from hydrogen, —F, —Cl, —NH₂, —CN, —OH, —NO₂, methyl, ethyl, n-propyl, i-propyl, ethenyl, propenyl, isopropenyl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)CH₃, —C(O)R¹⁵, —CO₂R¹⁶, —C(O)NR¹⁷, and —SO₂R¹⁸. In a still further aspect, each of R^{33a}, R^{33b}, and R³³ is independently selected from hydrogen, —F, —Cl, —NH₂, —CN, —OH, —NO₂, methyl, ethyl, ethenyl, —CH₂CN, —CH₂CH₂CN, —CH₂OH, —CH₂CH₂OH, —OCH₃, —OCH₂CH₃, —C(O)R¹⁵, —CO₂R¹⁶, —C(O)NR¹⁷, and —SO₂R¹⁸. In yet a further aspect, each of R^{33a}, R^{33b}, and R³³ is independently selected from hydrogen, —F, —Cl, —NH₂, —CN, —OH, —NO₂, methyl, —CH₂CN, —CH₂OH, —OCH₃, —C(O)R¹⁵, —CO₂R¹⁶, —C(O)NR¹⁷, and —SO₂R¹⁸.

In various aspects, each of R^{33a}, R^{33b}, and R^{33c} is independently selected from hydrogen, halogen, C1-C4 cyanoalkyl, C1-C4 hydroxyalkyl, and C1-C4 alkoxy. In a further aspect, each of R^{33a}, R^{33b}, and R³³ is independently selected from hydrogen, —F, —Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₃, —OCH₂CH₃, —OCH₂CH₂CH₃, and —OCH(CH₃)CH₃. In a still further aspect, each of R^{33a}, R^{33b}, and R^{33c} is independently selected from hydrogen, —F, —Cl, —CH₂CN, —CH₂CH₂CN, —CH₂OH, —CH₂CH₂OH, —OCH₃, and —OCH₂CH₃. In yet a further aspect, each of R^{33a}, R^{33b}, and R^{33c} is independently selected from hydrogen, —F, —Cl, —CH₂CN, —CH₂OH, and —OCH₃.

In various aspects, each of R^{33a}, R^{33b}, and R^{33c} is independently selected from hydrogen, halogen, —CN, —NH₂, —OH, —NO₂, C1-C4 alkyl, C2-C4 alkenyl, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In a further aspect, each of R^{33a}, R^{33b}, and R^{33c} is independently selected from hydrogen, —F, —Cl, —NH₂, —CN, —OH, —NO₂, methyl, ethyl, n-propyl, i-propyl, ethenyl, propenyl, isopropenyl, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, —CH(CH₃)CH₂NH₂, —C(O)R¹⁵, —CO₂R¹⁶, —C(O)NR¹⁷, and —SO₂R¹⁸. In a still further aspect, each of R^{33a}, R^{33b} and R^{33c} is independently selected from hydrogen, —F, —Cl, —NH₂, —CN, —OH, —NO₂, methyl, ethyl, ethenyl, —NHCH₃, —NHCH₂CH₃, —N(CH₃)₂, —N(CH₂CH₃)₂, —CH₂NH₂, —CH₂CH₂NH₂, —C(O)R¹⁵, —CO₂R¹⁶, —C(O)NR¹⁷, and —SO₂R¹⁸. In yet a further aspect, each of R^{33a}, R^{33b}, and R^{33c} is independently selected from hydrogen, —F, —Cl, —NH₂, —CN, —OH, —NO₂, methyl, —NHCH₃, —N(CH₃)₂, —CH₂NH₂, —C(O)R¹⁵, —CO₂R¹⁶, —C(O)NR¹⁷, and —S₂R¹⁸.

In various aspects, each of R^{33a}, R^{33b}, and R^{33c} is independently selected from hydrogen, halogen, C1-C4 alkylamino, (C1-C4)(C1-C4) dialkylamino, and C1-C4 aminoalkyl. In a further aspect, each of R^{33a}, R^{33b}, and R^{33c} is independently selected from hydrogen, —F, —Cl, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a still further aspect, each of R^{33a}, R^{33b}, and R^{33c} is independently selected from hydrogen, —F, —Cl, —NHCH₃, —NHCH₂CH₃, —N(CH₃)₂, —N(CH₂CH₃)₂, —CH₂NH₂, and —CH₂CH₂NH₂. In yet a further aspect, each of R^{33a}, R^{33b}, and R^{33c} is independently selected from hydrogen, —F, —Cl, —NHCH₃, —N(CH₃)₂, and —CH₂NH₂.

In various aspects, each of R^{33a}, R^{33b}, and R^{33c} is independently selected from hydrogen, halogen, —CN, —NH₂, —OH, —NO₂, C1-C4 alkyl, C2-C4 alkenyl, —C(O)R¹⁵, —CO₂R¹⁶, —C(O)NR¹⁷, and —SO₂R¹⁸. In a further aspect, each of R^{33a}, R^{33b}, and R^{33c} is independently selected from hydrogen, —F, —Cl, —NH₂, —CN, —OH, —NO₂, methyl, ethyl, n-propyl, i-propyl, ethenyl, propenyl, isopropenyl, —C(O)R¹⁵, —CO₂R¹⁶, —C(O)NR¹⁷, and —SO₂R¹⁸. In a still further aspect, each of R^{33a}, R^{33b}, and R^{33c} is independently selected from hydrogen, —F, —Cl, —NH₂, —CN, —OH, —NO₂, methyl, ethyl, ethenyl, —C(O)R¹⁵, —CO₂R¹⁶, —C(O)NR¹⁷, and —SO₂R¹⁸. In yet a further aspect, each of R^{33a}, R^{33b}, and R^{33c} is independently selected from hydrogen, —F, —Cl, —NH₂, —CN, —OH, —NO₂, methyl, —C(O)R¹⁵, —CO₂R¹⁶, —C(O)NR¹⁷, and —SO₂R¹⁸.

In various aspects, each of R^{33a}, R^{33b}, and R^{33c} is independently selected from hydrogen, halogen, C1-C4 alkyl, and C2-C4 alkenyl. In a further aspect, each of R^{33a}, R^{33b}, and R^{33c} is independently selected from hydrogen, —F, —Cl, methyl, ethyl, n-propyl, i-propyl, ethenyl, propenyl, and isopropenyl. In a still further aspect, each of R^{33a}, R^{33b}, and R^{33c} is independently selected from hydrogen, —F, —Cl, methyl, ethyl, and ethenyl. In yet a further aspect, each of R^{33a}, R^{33b}, and R³³ is independently selected from hydrogen, —F, —Cl, and methyl.

In a further aspect, each of R^{33a}, R^{33b}, and R^{33c} is independently selected from hydrogen, halogen, —CN, —NH₂, —OH, —NO₂, —C(O)R¹⁵, —CO₂R¹⁶, —C(O)NR¹⁷, and —SO₂R¹⁸.

In a further aspect, each of R^{33a}, R^{33b}, and R^{33c} is hydrogen.

j. R³⁴ Groups

In one aspect, R³⁴ is selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In a further aspect, R³⁴ is selected from —F, —Cl, —NH₂, —CN, —OH, —NO₂, methyl, ethyl, n-propyl, i-propyl, ethenyl, propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —SCF₃, —SCH₂CF₃, —SCH₂CH₂CF₃, —SCH(CH₃)CF₃, —SCH₃, —SCH₂CH₃, —SCH₂CH₂CH₃, —SCH(CH₃)CH₃, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, —CH(CH₃)CH₂NH₂, —C(O)R¹⁵, —CO₂R¹⁶, —C(O)NR¹⁷, and —SO₂R¹⁸. In a still further aspect, R³⁴ is selected from —F, —Cl, —NH₂, —CN, —OH, —NO₂, methyl, ethyl, ethenyl, —CH₂F, —CH₂Cl, —CH₂CH₂F, —CH₂CH₂Cl, —CH₂CN, —CH₂CH₂CN, —CH₂OH, —CH₂CH₂OH, —SCF₃, —SCH₂CF₃, —SCH₃, —SCH₂CH₃, —OCF₃, —OCH₂CF₃, —OCH₃, —OCH₂CH₃, —NHCH₃, —NHCH₂CH₃, —N(CH₃)₂, —N(CH₂CH₃)₂, —CH₂NH₂, —CH₂CH₂NH₂, —C(O)R¹⁵, —CO₂R¹⁶, —C(O)NR¹⁷, and —SO₂R¹⁸. In yet a further aspect, R³⁴ is selected from —F, —Cl, —NH₂, —CN, —OH, —NO₂, methyl, —CH₂F, —CH₂Cl, —CH₂CN, —CH₂OH, —SCF₃, —SCH₃, —OCF₃, —OCH₃, —NHCH₃, —N(CH₃)₂, —CH₂NH₂, —C(O)R¹⁵, —CO₂R¹⁶, —C(O)NR¹⁷, and —SO₂R¹⁸.

In various aspects, R³⁴ is selected from halogen, —CN, —NH₂, —OH, —NO₂, C1-C4 thioalkyl, C1-C4 thiohaloalkyl, —C(O)R¹⁵, —CO₂R¹⁶, —C(O)NR¹⁷, and —SO₂R¹⁸. In a further aspect, R³⁴ is selected from —F, —Cl, —NH₂, —CN, —OH, —NO₂, —SCF₃, —SCH₂CF₃, —SCH₂CH₂CF₃, —SCH(CH₃)CF₃, —SCH₃, —SCH₂CH₃, —SCH₂CH₂CH₃, —SCH(CH₃)CH₃, —C(O)R¹⁵, —CO₂R¹⁶, —C(O)NR¹⁷, and —SO₂R¹⁸. In a still further aspect, R³⁴ is selected from —F, —Cl, —NH₂, —CN, —OH, —NO₂, —SCF₃, —SCH₂CF₃, —SCH₃, —SCH₂CH₃, —C(O)R¹⁵, —CO₂R¹⁶, —C(O)NR¹⁷, and —S₂R¹⁸. In yet a further aspect, R³⁴ is selected from —F, —Cl, —NH₂, —CN, —OH, —NO₂, —SCF₃, —SCH₃, —C(O)R¹⁵, —CO₂R¹⁶, —C(O)NR¹⁷, and —S₂R¹⁸.

In various aspects, R³⁴ is selected from halogen, C1-C4 thioalkyl, and C1-C4 thiohaloalkyl. In a further aspect, R³⁴ is selected from —F, —Cl, —SCF₃, —SCH₂CF₃, —SCH₂CH₂CF₃, —SCH(CH₃)CF₃, —SCH₃, —SCH₂CH₃, —SCH₂CH₂CH₃, and —SCH(CH₃)CH₃. In a still further aspect, R³⁴ is selected from —F, —Cl, —SCF₃, —SCH₂CF₃, —SCH₃, and —SCH₂CH₃. In yet a further aspect, R³⁴ is selected from —F, —Cl, —SCF₃, and —SCH₃.

In various aspects, R³⁴ is selected from halogen, —CN, —NH₂, —OH, —NO₂, C1-C4 alkyl, C2-C4 alkenyl, C1-C4 haloalkyl, C1-C4 haloalkoxy, —C(O)R¹⁵, —CO₂R¹⁶, —C(O)NR¹⁷, and —SO₂R¹⁸. In a further aspect, R³⁴ is selected from —F, —Cl, —NH₂, —CN, —OH, —NO₂, methyl, ethyl, n-propyl, i-propyl, ethenyl, propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCF₃, —OCH₂CF₃, —OCH₂CH₂CF₃, —OCH(CH₃)CF₃, —C(O)R¹⁵, —CO₂R¹⁶, —C(O)NR¹⁷, and —SO₂R¹⁸. In a still further aspect, R³⁴ is selected from —F, —Cl, —NH₂, —CN, —OH, —NO₂, methyl, ethyl, ethenyl, —CH₂F, —CH₂Cl, —CH₂CH₂F, —CH₂CH₂Cl, —OCF₃, —OCH₂CF₃, —OCH₃, —OCH₂CH₃, —C(O)R¹⁵, —CO₂R¹⁶, —C(O)NR¹⁷, and —SO₂R¹⁸. Inyet a further aspect, R³⁴ is selected from —F, —Cl, —NH₂, —CN, —OH, —NO₂, methyl, —CH₂F, —CH₂Cl, —OCF₃, —C(O)R¹⁵, —CO₂R¹⁶, —C(O)NR¹⁷, and —SO₂R¹⁸.

In various aspects, R³⁴ is selected from halogen, C1-C4 haloalkyl, and C1-C4 haloalkoxy. In a further aspect, R³⁴ is selected from —F, —Cl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCF₃, —OCH₂CF₃, —OCH₂CH₂CF₃, and —OCH(CH₃)CF₃. In a still further aspect, R³⁴ is selected from —F, —Cl, —CH₂F, —CH₂C, —CH₂CH₂F, —CH₂CH₂C, —OCF₃, and —OCH₂CF₃. In yet a further aspect, R³⁴ is selected from —F, —Cl, —CH₂F, —CH₂Cl, and —OCF₃.

In various aspects, R³⁴ is selected from halogen, —CN, —NH₂, —OH, —NO₂, C1-C4 alkyl, C2-C4 alkenyl, C1-C4 cyanoalkyl, C1-C4 hydroxyalkyl, C1-C4 alkoxy, —C(O)R¹⁵, —CO₂R¹⁶, —C(O)NR¹⁷, and —SO₂R¹⁸. In a further aspect, R³⁴ is selected from —F, —Cl, —NH₂, —CN, —OH, —NO₂, methyl, ethyl, n-propyl, i-propyl, ethenyl, propenyl, isopropenyl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)CH₃, —C(O)R¹⁵, —CO₂R¹⁶, —C(O)NR¹⁷, and —SO₂R¹⁸. In a still further aspect, R³⁴ is selected from —F, —Cl, —NH₂, —CN, —OH, —NO₂, methyl, ethyl, ethenyl, —CH₂CN, —CH₂CH₂CN, —CH₂OH, —CH₂CH₂OH, —OCH₃, —OCH₂CH₃, —C(O)R¹⁵, —CO₂R¹⁶, —C(O)NR¹⁷, and —SO₂R¹⁸. In yet a further aspect, R³⁴ is selected from —F, —Cl, —NH₂, —CN, —OH, —NO₂, methyl, —CH₂CN, —CH₂OH, —OCH₃, —C(O)R¹⁵, —CO₂R¹⁶, —C(O)NR¹⁷, and —SO₂R¹⁸.

In various aspects, R³⁴ is selected from halogen, C1-C4 cyanoalkyl, C1-C4 hydroxyalkyl, and C1-C4 alkoxy. Ina further aspect, R³⁴ is selected from —F, —Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₃, —OCH₂CH₃, —OCH₂CH₂CH₃, and —OCH(CH₃)CH₃. In a still further aspect, R³⁴ is selected from —F, —Cl, —CH₂CN, —CH₂CH₂CN, —CH₂OH, —CH₂CH₂H, —OCH₃, and —OCH₂CH₃. In yet a further aspect, R³⁴ is selected from —F, —Cl, —CH₂CN, —CH₂OH, and —OCH₃.

In various aspects, R³⁴ is selected from halogen, —CN, —NH₂, —OH, —NO₂, C1-C4 alkyl, C2-C4 alkenyl, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In a further aspect, R³⁴ is selected from —F, —Cl, —NH₂, —CN, —OH, —NO₂, methyl, ethyl, n-propyl, i-propyl, ethenyl, propenyl, isopropenyl, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, —CH(CH₃)CH₂NH₂, —C(O)R¹⁵, —CO₂R¹⁶, —C(O)NR¹⁷, and —SO₂R¹⁸. In a still further aspect, R³⁴ is selected from —F, —Cl, —NH₂, —CN, —OH, —NO₂, methyl, ethyl, ethenyl, —NHCH₃, —NHCH₂CH₃, —N(CH₃)₂, —N(CH₂CH₃)₂, —CH₂NH₂, —CH₂CH₂NH₂, —C(O)R¹⁵, —CO₂R¹⁶, —C(O)NR¹⁷, and —SO₂R¹⁸. In yet a further aspect, R³⁴ is selected from —F, —Cl, —NH₂, —CN, —OH, —NO₂, methyl, —NHCH₃, —N(CH₃)₂, —CH₂NH₂, —C(O)R¹⁵, —CO₂R¹⁶, —C(O)NR¹⁷, and —SO₂R¹⁸.

In various aspects, R³⁴ is selected from halogen, C1-C4 alkylamino, (C1-C4)(C1-C4) dialkylamino, and C1-C4 aminoalkyl. In a further aspect, R³⁴ is selected from —F, —Cl, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a still further aspect, R³⁴ is selected from —F, —Cl, —NHCH₃, —NHCH₂CH₃, —N(CH₃)₂, —N(CH₂CH₃)₂, —CH₂NH₂, and —CH₂CH₂NH₂. In yet a further aspect, R³⁴ is selected from —F, —Cl, —NHCH₃, —N(CH₃)₂, and —CH₂NH₂.

In various aspects, R³⁴ is selected from halogen, —CN, —NH₂, —OH, —NO₂, C1-C4 alkyl, C2-C4 alkenyl, —C(O)R¹⁵, —CO₂R¹⁶, —C(O)NR¹⁷, and —SO₂R¹⁸. In a further aspect, R³⁴ is selected from —F, —Cl, —NH₂, —CN, —OH, —NO₂, methyl, ethyl, n-propyl, i-propyl, ethenyl, propenyl, isopropenyl, —C(O)R¹⁵, —CO₂R¹⁶, —C(O)NR¹⁷, and —SO₂R¹⁸. In a still further aspect, R³⁴ is selected from —F, —Cl, —NH₂, —CN, —OH, —NO₂, methyl, ethyl, ethenyl, —C(O)R¹⁵, —CO₂R¹⁶, —C(O)NR¹⁷, and —SO₂R¹⁸. In yet a further aspect, R³⁴ is selected from —F, —Cl, —NH₂, —CN, —OH, —NO₂, methyl, —C(O)R¹⁵, —CO₂R¹⁶, —C(O)NR¹⁷, and —SO₂R¹⁸.

In various aspects, R³⁴ is selected from halogen, C1-C4 alkyl, and C2-C4 alkenyl. In a further aspect, R³⁴ is selected from —F, —Cl, methyl, ethyl, n-propyl, i-propyl, ethenyl, propenyl, and isopropenyl. In a still further aspect, R³⁴ is selected from —F, —Cl, methyl, ethyl, and ethenyl. In yet a further aspect, R³⁴ is selected from —F, —Cl, and methyl.

In a further aspect, R³⁴ is selected from halogen, —CN, —NH₂, —OH, —NO₂, —C(O)R¹⁵, —CO₂R¹⁶, —C(O)NR¹⁷, and —SO₂R¹⁸.

In a further aspect, R³⁴ is selected from halogen, C1-C4 alkyl, C1-C4 haloalkyl, C1-C4 cyanoalkyl, C1-C4 thioalkyl, C1-C4 thiohaloalkyl, C1-C4 alkoxy, and —CO₂R¹⁶. In a still further aspect, R³⁴ is selected from —F, —Cl, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —SCF₃, —SCH₂CF₃, —SCH₂CH₂CF₃, —SCH(CH₃)CF₃, —SCH₃, —SCH₂CH₃, —SCH₂CH₂CH₃, —SCH(CH₃)CH₃, —OCH₃, —OCH₂CH₃, —OCH₂CH₂CH₃, —OCH(CH₃)CH₃, and —CO₂R¹⁶. In yet a further aspect, R³⁴ is selected from —F, —Cl, methyl, ethyl, —CH₂F, —CH₂Cl, —CH₂CH₂F, —CH₂CH₂Cl, —CH₂CN, —CH₂CH₂CN, —SCF₃, —SCH₂CF₃, —SCH₃, —SCH₂CH₃, —OCH₃, —OCH₂CH₃, and —CO₂R¹⁶. In an even further aspect, R³⁴ is selected from —F, —Cl, methyl, —CH₂F, —CH₂Cl, —CH₂CN, —SCF₃, —SCH₃, —OCH₃, and —CO₂R¹⁶.

In a further aspect, R³⁴ is C1-C4 thioalkyl. In a still further aspect, R³⁴ is selected from —SCH₃, —SCH₂CH₃, —SCH₂CH₂CH₃, and —SCH(CH₃)CH₃. In yet a further aspect, R³⁴ is selected from —SCH₃ and —SCH₂CH₃. In an even further aspect, R³⁴ is —SCH₃.

k. Cy¹ Groups

In one aspect, Cy², when present, is selected from 5-membered heterocycle and 6-membered heterocycle, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl. In a further aspect, Cy¹, when present, is selected from 5-membered heterocycle and 6-membered heterocycle, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy¹, when present, is selected from 5-membered heterocycle and 6-membered heterocycle, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹, when present, is selected from 5-membered heterocycle and 6-membered heterocycle, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹, when present, is selected from 5-membered heterocycle and 6-membered heterocycle, and is unsubstituted.

In various aspects, Cy¹, when present, is 5-membered heterocycle substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl. Examples of 5-membered heterocycles include, but are not limited to, pyrrolidine, thiolane, and tetrahydrofuran. In a further aspect, Cyl, when present, is 5-membered heterocycle substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl. In a still further aspect, Cyi, when present, is 5-membered heterocycle substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹, when present, is 5-membered heterocycle monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹, when present, is unsubstituted 5-membered heterocycle.

In various aspects, Cy¹, when present, is 6-membered heterocycle substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl. Examples of 6-membered heterocycles include, but are not limited to, piperidine, tetrahydropyran, morpholine, 1,4-dioxane, and piperazine. In a further aspect, Cy¹, when present, is 6-membered heterocycle substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl. Ina still further aspect, Cyi, when present, is 6-membered heterocycle substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹, when present, is 6-membered heterocycle monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹, when present, is unsubstituted 6-membered heterocycle.

In various aspects, Cy¹, when present, is morpholinyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl. In a further aspect, Cy¹, when present, is morpholinyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy¹, when present, is morpholinyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹, when present, is morpholinyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹, when present, is unsubstituted morpholinyl.

l. Ar¹ Groups

In one aspect, Ar¹ is selected from pyridinyl, piperazinyl, and 6-membered aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴. In a further aspect, Ar¹ is selected from pyridinyl, piperazinyl, and 6-membered aryl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴. In a still further aspect, Ar¹ is selected from pyridinyl, piperazinyl, and 6-membered aryl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴. In yet a further aspect, Ar¹ is selected from pyridinyl, piperazinyl, and 6-membered aryl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴. In an even further aspect, Ar¹ is selected from pyridinyl, piperazinyl, and 6-membered aryl, and is unsubstituted.

In one aspect, Ar¹ is a structure represented by a formula:

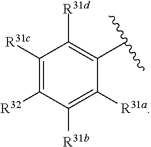

In various aspects, Ar¹ is selected from pyridinyl and piperazinyl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴. In a further aspect, Ar¹ is selected from pyridinyl and piperazinyl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹, —CO₂R¹², —C(O)NR¹³, and —SO₂R¹⁴. In a still further aspect, Ar is selected from pyridinyl and piperazinyl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴. In yet a further aspect, Ar¹ is selected from pyridinyl and piperazinyl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴. In an even further aspect, Ar¹ is selected from pyridinyl and piperazinyl, and is unsubstituted.

In various aspects, Ar¹ is pyridinyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴. In a further aspect, Ar¹ is pyridinyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R, —CO₂R¹², —C(O)NR¹³, and —SO₂R¹⁴. In a still further aspect, Ar¹ is pyridinyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴. In yet a further aspect, Ar¹ is pyridinyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴. In an even further aspect, Ar¹ is pyridinyl unsubstituted.

In a further aspect, Ar¹ is pyridinyl para-substituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴.

In a further aspect, Ar¹ is pyridinyl monosubstituted with a —CN group. In a still further aspect, Ar¹ is pyridinyl para-substituted with a —CN group. In yet a further aspect, Ar¹ is pyridinyl meta-substituted with a —CN group. In an even further aspect, Ar¹ is pyridinyl ortho-substituted with a —CN group.

In various aspects, Ar¹ is piperazinyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴. In a further aspect, Ar¹ is piperazinyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R, —CO₂R¹², —C(O)NR¹³, and —SO₂R¹⁴. In a still further aspect, Ar¹ is piperazinyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R, —CO₂R¹², —C(O)NR¹³, and —SO₂R¹⁴. In yet a further aspect, Ar¹ is piperazinyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴. In an even further aspect, Ar¹ is piperazinyl unsubstituted.

In a further aspect, Ar¹ is piperazinyl para-substituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹, —CO₂R¹², —C(O)NR¹³, and —SO₂R¹⁴.

In a further aspect, Ar¹ is piperazinyl monosubstituted with a —CN group. In a still further aspect, Ar¹ is piperazinyl para-substituted with a —CN group. In yet a further aspect, Ar¹ is piperazinyl meta-substituted with a —CN group. In an even further aspect, Ar is piperazinyl ortho-substituted with a —CN group.

In various aspects, Ar¹ is 6-membered aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴. In a further aspect, Ar¹ is 6-membered aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴. In a still further aspect, Ar¹ is 6-membered aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —S₂R¹⁴. In yet a further aspect, Ar¹ is 6-membered aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R, —CO₂R¹², —C(O)NR¹³, and —SO₂R¹⁴. In an even further aspect, Ar¹ is 6-membered aryl unsubstituted.

In a further aspect, Ar¹ is 6-membered aryl para-substituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴.

In a further aspect, Ar¹ is 6-membered aryl monosubstituted with a —CN group. In a still further aspect, Ar¹ is 6-membered aryl para-substituted with a —CN group. In yet a further aspect, Ar¹ is 6-membered aryl meta-substituted with a —CN group. In an even further aspect, Ar¹ is 6-membered aryl ortho-substituted with a —CN group.

In a further aspect, Ar¹ is a structure represented by a formula:

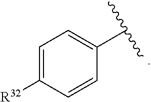

m. AR² GROUPS

In one aspect, Ar² is selected from 5-membered heterocycle, 6-membered heterocycle, 5-membered heteroaryl, and 6-membered heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In a further aspect, Ar² is selected from 5-membered heterocycle, 6-membered heterocycle, 5-membered heteroaryl, and 6-membered heteroaryl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In a still further aspect, Ar² is selected from 5-membered heterocycle, 6-membered heterocycle, 5-membered heteroaryl, and 6-membered heteroaryl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In yet a further aspect, Ar² is selected from 5-membered heterocycle, 6-membered heterocycle, 5-membered heteroaryl, and 6-membered heteroaryl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In an even further aspect, Ar² is selected from 5-membered heterocycle, 6-membered heterocycle, 5-membered heteroaryl, and 6-membered heteroaryl, and is unsubstituted.

In one aspect, Ar² is selected from 5-membered heterocycle, 6-membered heterocycle, 5-membered heteroaryl, 6-membered heteroaryl, 6-membered aryl, and bicyclo[1.1.1]pentanyl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In a further aspect, Ar² is selected from 5-membered heterocycle, 6-membered heterocycle, 5-membered heteroaryl, 6-membered heteroaryl, 6-membered aryl, and bicyclo[1.1.1]pentanyl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In a still further aspect, Ar² is selected from 5-membered heterocycle, 6-membered heterocycle, 5-membered heteroaryl, 6-membered heteroaryl, 6-membered aryl, and bicyclo[1.1.1]pentanyl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In yet a further aspect, Ar² is selected from 5-membered heterocycle, 6-membered heterocycle, 5-membered heteroaryl, 6-membered heteroaryl, 6-membered aryl, and bicyclo[1.1.1]pentanyl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In an even further aspect, Ar² is selected from 5-membered heterocycle, 6-membered heterocycle, 5-membered heteroaryl, 6-membered heteroaryl, 6-membered aryl, and bicyclo[1.1.1]pentanyl, and is unsubstituted.

In various aspects, Ar² is selected from 5-membered heterocycle and 6-membered heterocycle, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In a further aspect, Ar² is selected from 5-membered heterocycle and 6-membered heterocycle, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In a still further aspect, Ar² is selected from 5-membered heterocycle and 6-membered heterocycle, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In yet a further aspect, Ar² is selected from 5-membered heterocycle and 6-membered heterocycle, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In an even further aspect, Ar² is selected from 5-membered heterocycle and 6-membered heterocycle, and is unsubstituted.

In various aspects, Ar² is 5-membered heterocycle substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. Examples of 5-membered heterocycles include, but are not limited to, pyrrolidine, thiolane, and tetrahydrofuran. In a further aspect, Ar² is 5-membered heterocycle substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In a still further aspect, Ar² is 5-membered heterocycle substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In yet a further aspect, Ar² is 5-membered heterocycle monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In an even further aspect, Ar² is unsubstituted 5-membered heterocycle.

In various aspects, Ar² is 6-membered heterocycle substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. Examples of 6-membered heterocycles include, but are not limited to, piperidine, tetrahydropyran, morpholine, 1,4-dioxane, and piperazine. In a further aspect, Ar² is 6-membered heterocycle substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In a still further aspect, Ar² is 6-membered heterocycle substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In yet a further aspect, Ar² is 6-membered heterocycle monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In an even further aspect, Ar² is unsubstituted 6-membered heterocycle.

In various aspects, Ar² is selected from 5-membered heteroaryl, 6-membered heteroaryl, and 6-membered aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In a further aspect, Ar² is selected from 5-membered heteroaryl, 6-membered heteroaryl, and 6-membered aryl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In a still further aspect, Ar² is selected from 5-membered heteroaryl, 6-membered heteroaryl, and 6-membered aryl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In yet a further aspect, Ar² is selected from 5-membered heteroaryl, 6-membered heteroaryl, and 6-membered aryl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In an even further aspect, Ar² is selected from 5-membered heteroaryl, 6-membered heteroaryl, and 6-membered aryl, and is unsubstituted.

In various aspects, Ar² is selected from 5-membered heteroaryl and 6-membered heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In a further aspect, Ar² is selected from 5-membered heteroaryl and 6-membered heteroaryl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In a still further aspect, Ar² is selected from 5-membered heteroaryl and 6-membered heteroaryl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In yet a further aspect, Ar² is selected from 5-membered heteroaryl and 6-membered heteroaryl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸. In an even further aspect, Ar² is selected from 5-membered heteroaryl and 6-membered heteroaryl, and is unsubstituted.

In various aspects, Ar² is 5-membered heteroaryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. Examples of 5-membered heteroaryls include, but are not limited to, pyrrole, furan, thiophene, oxazole, isoxazole, isothiazole, and thiazole. In a further aspect, Ar² is 5-membered heteroaryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In a still further aspect, Ar² is 5-membered heteroaryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. Inyet a further aspect, Ar² is 5-membered heteroaryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In an even further aspect, Ar² is unsubstituted 5-membered heteroaryl.

In various aspects, Ar² is 6-membered heteroaryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. Examples of 6-membered heteroaryls include, but are not limited to, pyridine, pyridazine, pyrimidine, 1,2,4-triazole, and 1,3,5-triazole. In a further aspect, Ar² is 6-membered heteroaryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In a still further aspect, Ar² is 6-membered heteroaryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In yet a further aspect, Ar² is 6-membered heteroaryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In an even further aspect, Ar² is unsubstituted 6-membered heteroaryl.

In various aspects, Ar² is pyridinyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In a further aspect, Ar² is pyridinyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In a still further aspect, Ar² is pyridinyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In yet a further aspect, Ar² is pyridinyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In an even further aspect, Ar² is unsubstituted pyridinyl.

In various aspects, Ar² is 6-membered aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In a further aspect, Ar² is 6-membered aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In a still further aspect, Ar² is 6-membered aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In yet a further aspect, Ar² is 6-membered aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In an even further aspect, Ar² is unsubstituted 6-membered aryl.

In various aspects, Ar² is bicyclo[1.1.1]pentanyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In a further aspect, Ar² is bicyclo[1.1.1]pentanyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In a still further aspect, Ar² is bicyclo[1.1.1]pentanyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In yet a further aspect, Ar² is bicyclo[0.1.1]pentanyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁶, —CO₂R¹⁶, —C(O)NR¹⁷, and —SO₂R¹⁸. In an even further aspect, Ar² is unsubstituted bicyclo[1.1.1]pentanyl.

In a further aspect, Ar² is a structure represented by a formula:

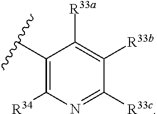

In a further aspect, Ar² is a structure represented by a formula:

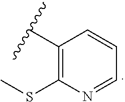 [SMILES: *c1cccnc1SC]

n. AR³ GROUPS

In one aspect, Ar³, when present, is phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar³, when present, is phenyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is phenyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar³, when present, is phenyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar³, when present, is unsubstituted phenyl.

2. Example Compounds

In one aspect, a compound can be present as one or more of the following structures:

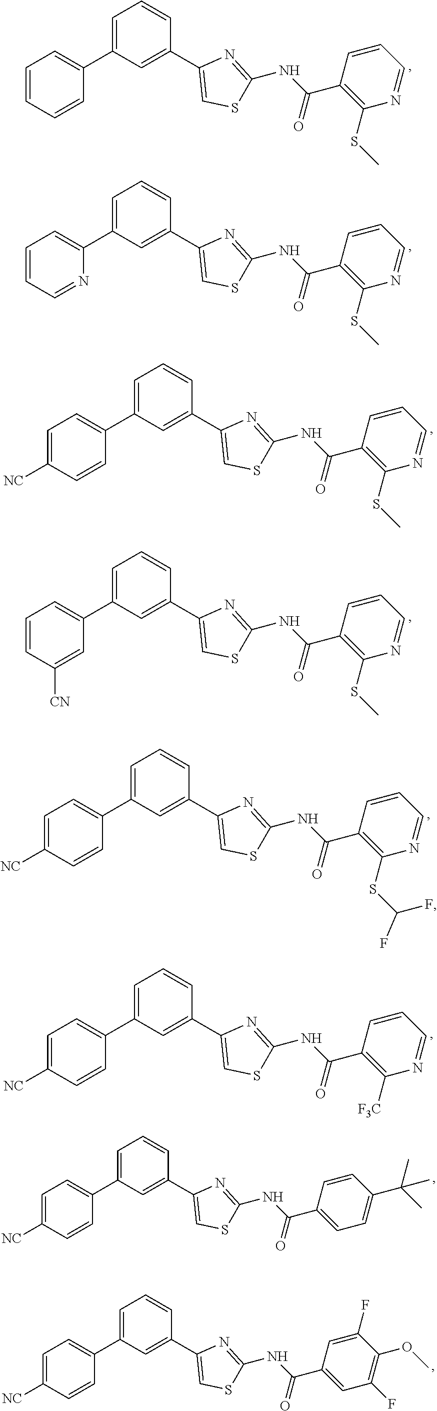

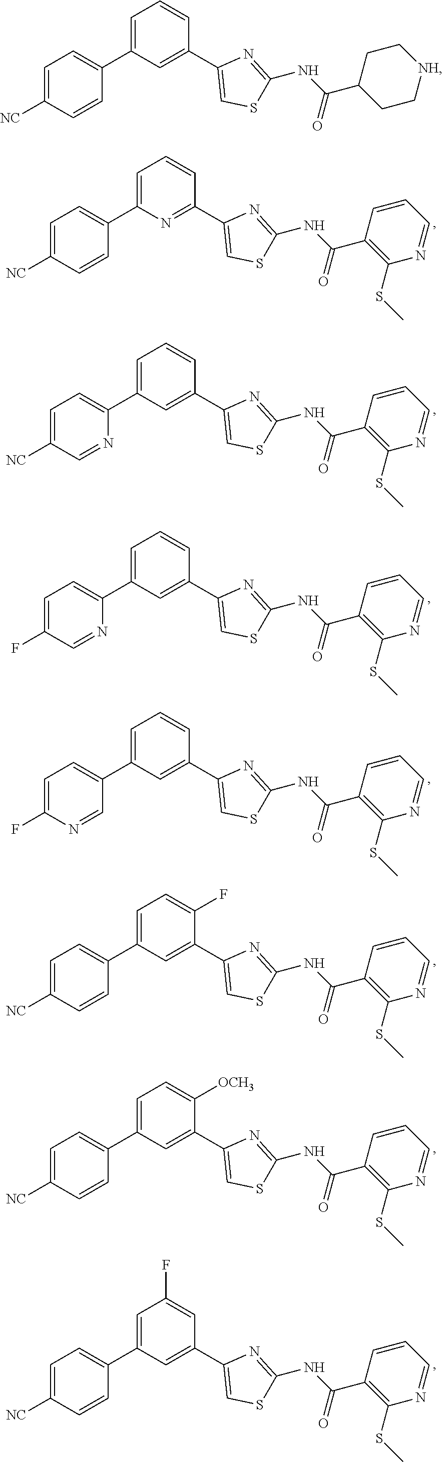

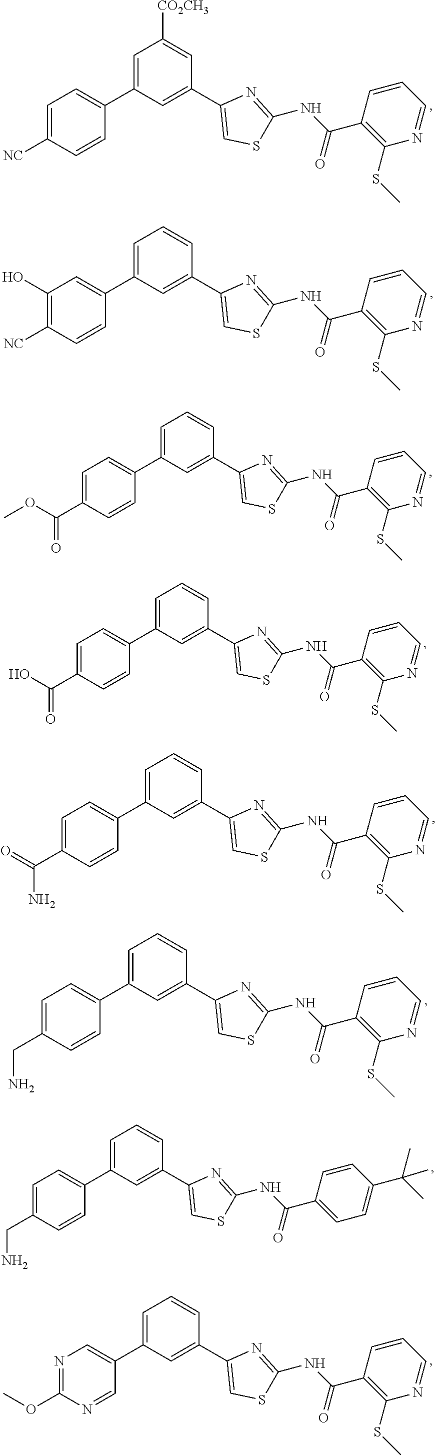

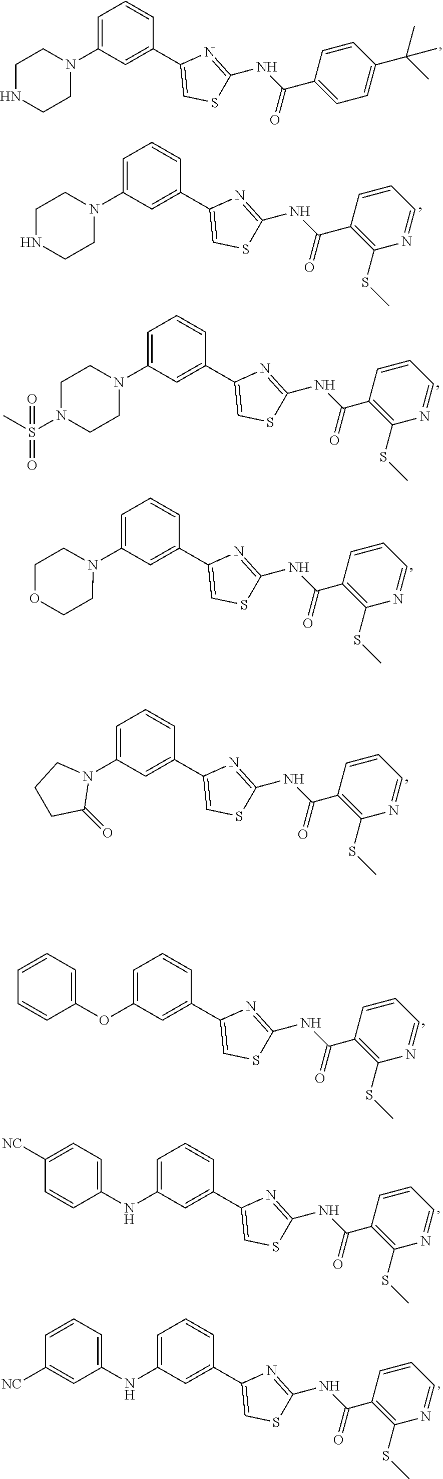

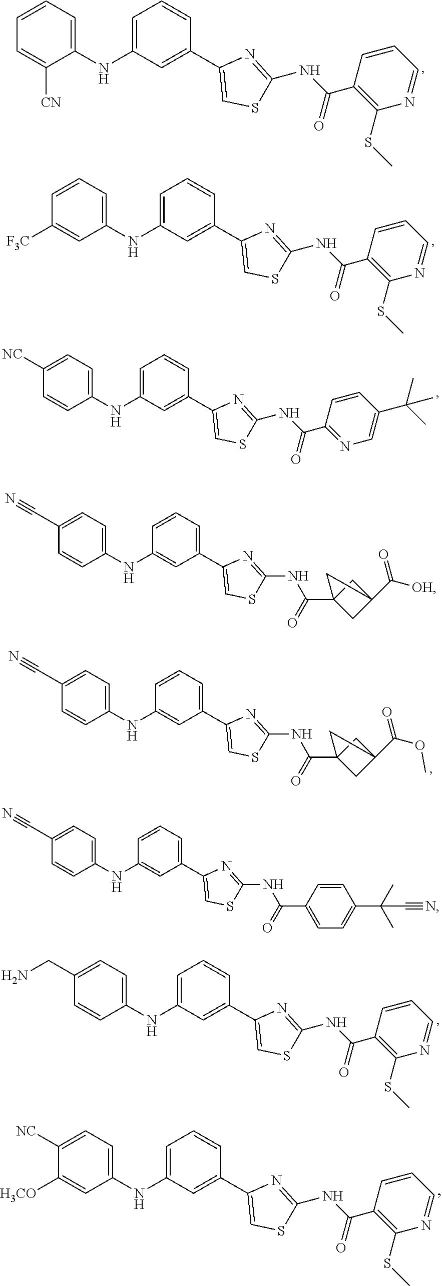

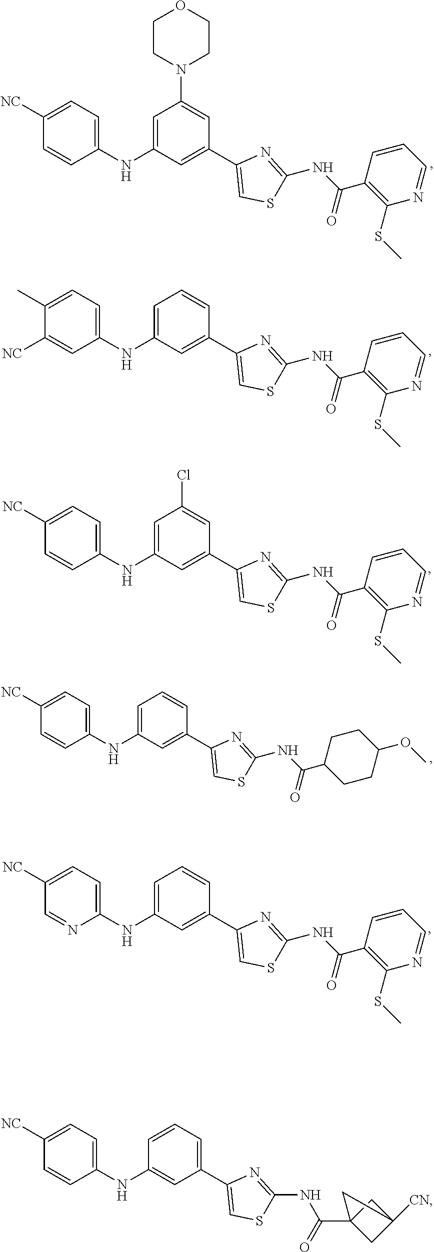 [SMILES: CSc1ncccc1C(=O)Nc1nc(-c2cc(Nc3ccc(C#N)cc3)cc(N3CCOCC3)c2)cs1 ; CSc1ncccc1C(=O)Nc1nc(-c2cccc(Nc3ccc(C)c(C#N)c3)c2)cs1 ; CSc1ncccc1C(=O)Nc1nc(-c2cc(Cl)cc(Nc3ccc(C#N)cc3)c2)cs1 ; COC1CCC(C(=O)Nc2nc(-c3cccc(Nc4ccc(C#N)cc4)c3)cs2)CC1 ; CSc1ncccc1C(=O)Nc1nc(-c2cccc(Nc3ccc(C#N)cn3)c2)cs1 ; N#Cc1ccc(Nc2cccc(-c3csc(NC(=O)C45CC(C#N)(C4)C5)n3)c2)cc1]

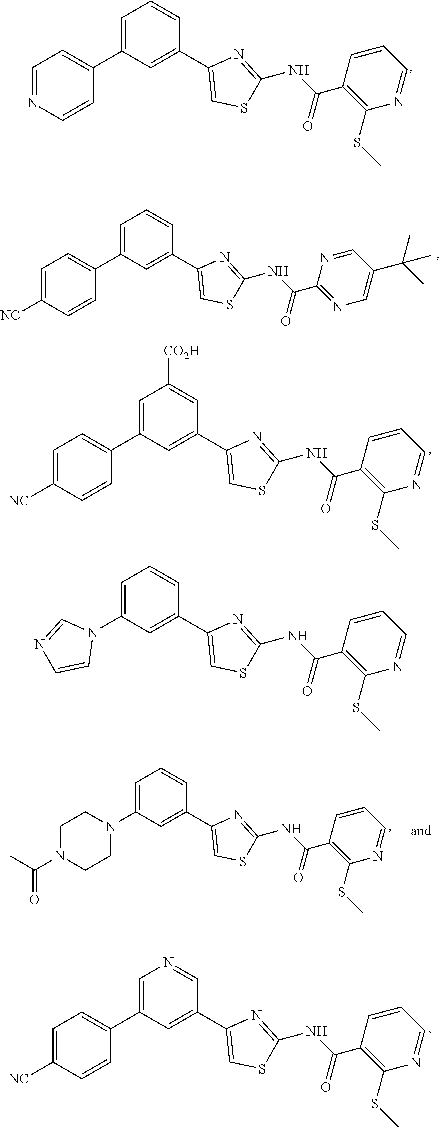

or a pharmaceutically acceptable salt thereof.

### C. PHARMACEUTICAL COMPOSITIONS

In one aspect, disclosed are pharmaceutical compositions comprising a disclosed compound, or a pharmaceutically acceptable salt thereof and a pharmaceutically acceptable carrier.

In one aspect, disclosed are pharmaceutical compositions comprising a pharmaceutically acceptable carrier and a therapeutically effective amount of at least one compound having a structure represented by a formula:

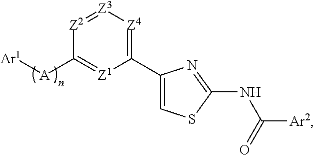

wherein n is selected from 0 and 1; wherein A, when present, is selected from O, NH, N(C1-C4 alkyl), and N(Ar³); wherein Ar³, when present, is phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of Z¹, Z², Z³, and Z⁴ is independently selected from N and CR¹⁰, provided that at least two of Z¹, Z², Z³, and Z⁴ are CR¹⁰; wherein each occurrence of R¹⁰, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², —SO₂R²³, and Cy¹; wherein each occurrence of R²⁰, R²¹, R²², and R²³, when present, is independently selected from hydrogen and C1-C4 alkyl; wherein Cy¹, when present, is selected from 5-membered heterocycle and 6-membered heterocycle, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl; and either: (a) wherein Ar¹ is selected from pyridinyl, piperazinyl, and 6-membered aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴; wherein each occurrence of R¹, R¹², R¹³, and R¹⁴, when present, is independently selected from hydrogen and C1-C4 alkyl; and wherein Ar² is selected from 5-membered heterocycle, 6-membered heterocycle, 5-membered heteroaryl, and 6-membered heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸; and wherein each occurrence of R¹, R¹⁶, R¹⁷, and R¹⁸, when present, is independently selected from hydrogen and C1-C4 alkyl; or (b) wherein Ar¹ is a structure represented by a formula:

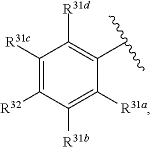

wherein each of R^{31a}, R^{31b}, R³1, and R^{31d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴; wherein R³² is selected from —CN and —CH₂NH₂; and wherein Ar² is selected from 5-membered heterocycle, 6-membered heterocycle, 5-membered heteroaryl, 6-membered heteroaryl, 6-membered aryl, and bicyclo[1.1.1]pentanyl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸, or a pharmaceutically acceptable salt thereof.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, e.g., administration by drops or injection into the ear, insufflation (such as into the ear), intravenous, topical, or oral administration.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, Pa. 1990.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier canbe a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouthwashes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, an effective amount is a therapeutically effective amount. In a still further aspect, an effective amount is a prophylactically effective amount.

In a further aspect, the pharmaceutical composition is administered to a mammal. In a still further aspect, the mammal is a human. In an even further aspect, the human is a patient.

In a further aspect, the pharmaceutical composition is used to treat a viral infection such as, for example, chikungunya, Venezuelan equine encephalitis, dengue, influenza, and zika.

It is understood that the disclosed compositions can be prepared from the disclosed compounds. It is also understood that the disclosed compositions can be employed in the disclosed methods of using.

### D. METHODS OF MAKING A COMPOUND

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-III, as described and exemplified below. The following examples are provided so that the invention might be more fully understood, are illustrative only, and should not be construed as limiting.

1. Route I

In one aspect, substituted biphenyl thiazoles can be prepared as shown below.

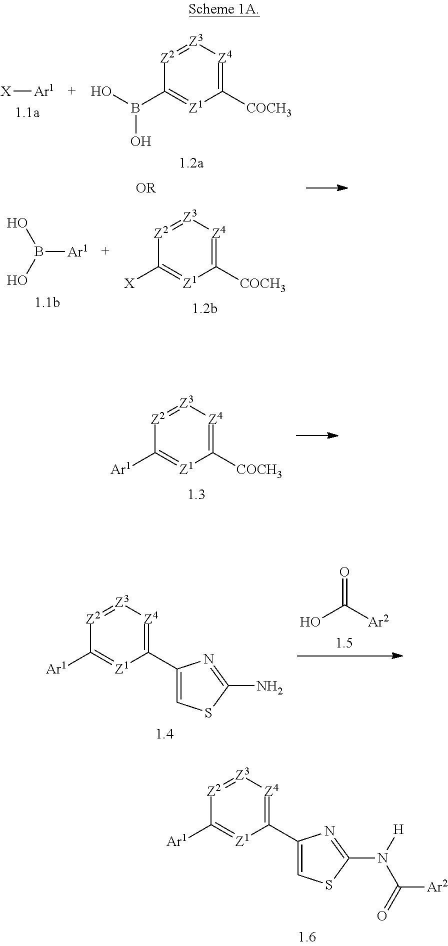

Compounds are represented in generic form, wherein X is a halogen and with substituents as noted in compound descriptions elsewhere herein. A more specific example is set forth below.

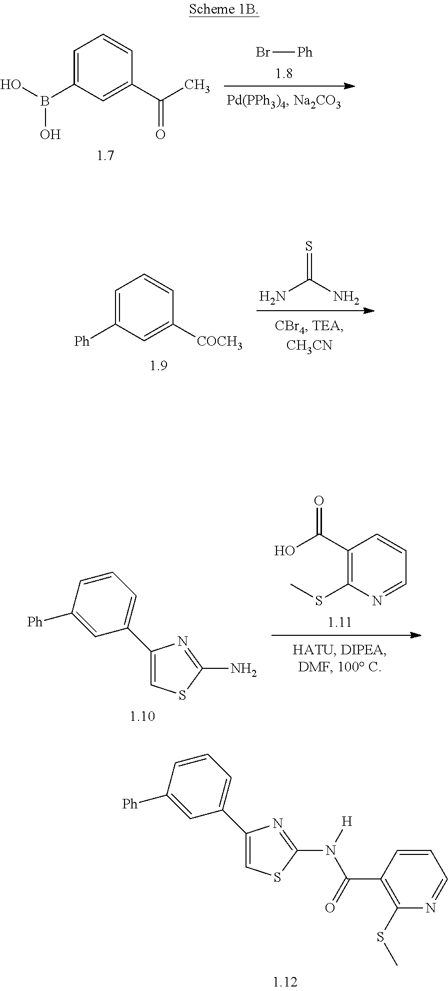

In one aspect, compounds of type 1.12, and similar compounds, can be prepared according to reaction Scheme 1B above. Thus, compounds of type 1.9 can be prepared by a coupling reaction between an appropriate aryl boronic acid, e.g., 1.7 as shown above, and an appropriate aryl halide, e.g., 1.8 as shown above. Appropriate aryl boronic acids and appropriate aryl halides are commercially available or prepared by methods known to one skilled in the art. The coupling reaction is carried out in the presence of an appropriate catalyst, e.g., tetrakis(triphenylphosphine)palladium(0), and an appropriate base, e.g., sodium carbonate. Compounds of type 1.10 can be prepared by a cyclization reaction of an appropriate acetophenone, e.g., 1.9 as shown above. The cyclization reaction is carried out in the presence of an appropriate activating agent, e.g., carbon tetrabromide, an appropriate thiourea, e.g., thiourea as shown above, and an appropriate base, e.g., triethylamine (TEA), in an appropriate solvent, e.g., acetonitrile. Compounds of type 1.12 can be prepared by a coupling reaction between an appropriate amine, e.g., 1.10 as shown above, and an appropriate carboxylic acid, e.g., 1.11 as shown above. Appropriate carboxylic acids are commercially available or prepared by methods known to one skilled in the art. The coupling reaction is carried out in the presence of an appropriate coupling agent, e.g., 1-[bis(dimethylamino)methylene]-1H-1,2,3-triazolo[4,5-b]pyridinium 3-oxid hexafluorophosphate (HATU), and an appropriate base, e.g., diisopropylethylamine (DIPEA), in an appropriate solvent, e.g., dimethylformamide, at an appropriate temperature, e.g., 100° C. As can be appreciated by one skilled in the art, the above reaction provides an example of a generalized approach wherein compounds similar in structure to the specific reactants above (compounds similar to compounds of type 1.1a, 1.1b, 1.2a, 1.2b, 1.3, 1.4, and 1.5), can be substituted in the reaction to provide substituted biphenyl thiazole derivatives similar to Formula 1.6.

2. Route II

In one aspect, substituted biphenyl thiazoles can be prepared as shown below.

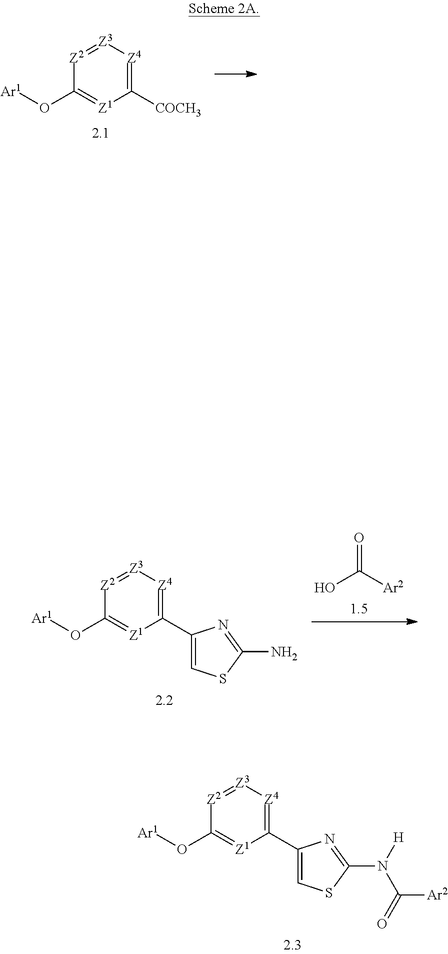

Compounds are represented in generic form, with substituents as noted in compound descriptions elsewhere herein. A more specific example is set forth below.

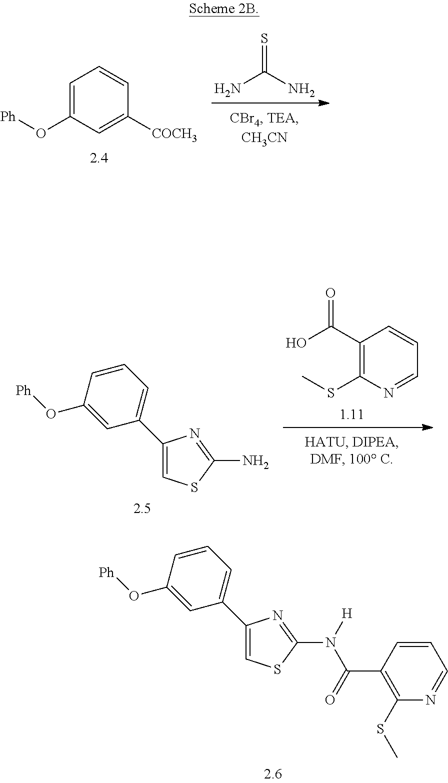

In one aspect, compounds of type 2.6, and similar compounds, can be prepared according to reaction Scheme 2B above. Thus, compounds of type 2.5 can be prepared by a cyclization reaction of an appropriate acetophenone, e.g., 2.4 as shown above. The cyclization reaction is carried out in the presence of an appropriate activating agent, e.g., carbon tetrabromide, an appropriate thiourea, e.g., thiourea as shown above, and an appropriate base, e.g., triethylamine (TEA), in an appropriate solvent, e.g., acetonitrile. Compounds of type 2.6 can be prepared by a coupling reaction between an appropriate amine, e.g., 2.5 as shown above, and an appropriate carboxylic acid, e.g., 1.11 as shown above. Appropriate carboxylic acids are commercially available or prepared by methods known to one skilled in the art. The coupling reaction is carried out in the presence of an appropriate coupling agent, e.g., 1-[bis(dimethylamino)methylene]-1H-1,2,3-triazolo[4,5-b]pyridinium 3-oxid hexafluorophosphate (HATU), and an appropriate base, e.g., diisopropylethylamine (DIPEA), in an appropriate solvent, e.g., dimethylformamide, at an appropriate temperature, e.g., 100° C. As can be appreciated by one skilled in the art, the above reaction provides an example of a generalized approach wherein compounds similar in structure to the specific reactants above (compounds similar to compounds of type 1.5, 2.1, and 2.2), can be substituted in the reaction to provide substituted biphenyl thiazole derivatives similar to Formula 2.3.

3. Route III

In one aspect, substituted biphenyl thiazoles can be prepared as shown below.

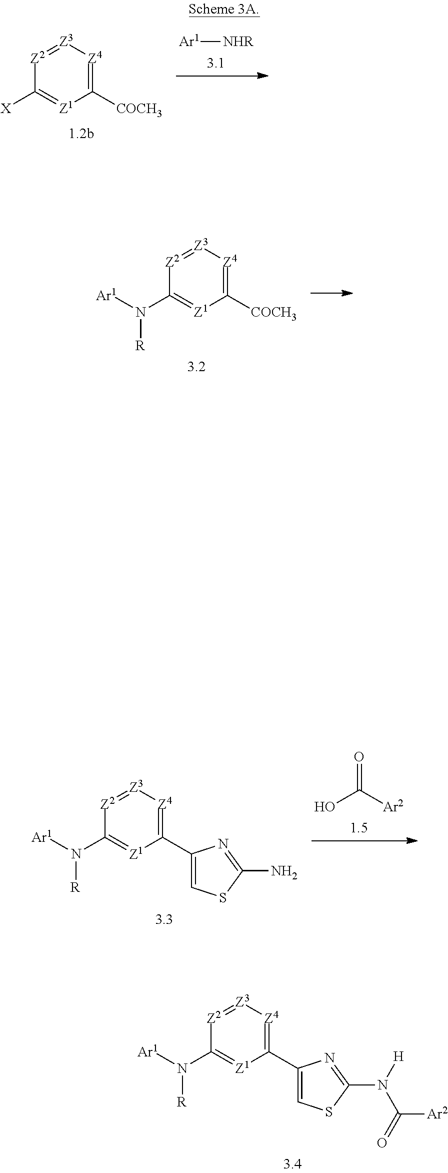

Compounds are represented in generic form, wherein X is a halogen, wherein R is hydrogen, C1-C4 alkyl, or Ar³, and with substituents as noted in compound descriptions elsewhere herein. A more specific example is set forth below.

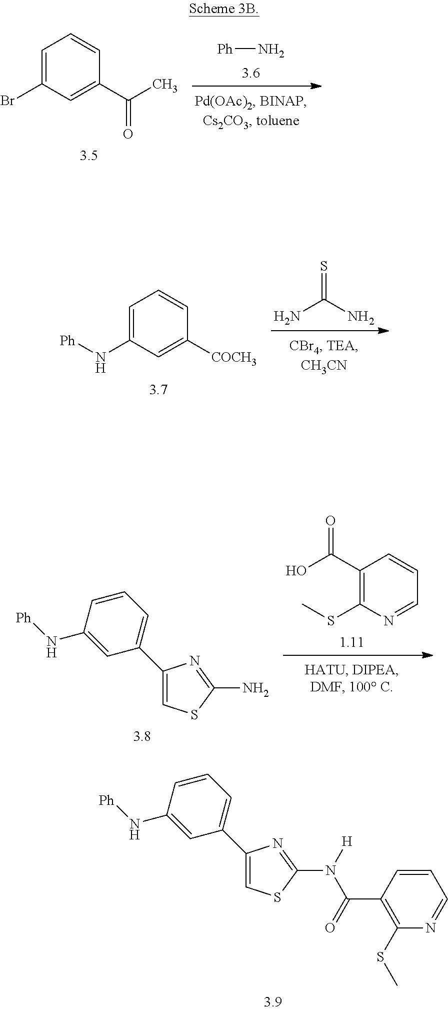

In one aspect, compounds of type 3.9, and similar compounds, can be prepared according to reaction Scheme 3B above. Thus, compounds of type 3.7 can be prepared by a coupling reaction between an appropriate acetophenone halides, e.g., 3.5 as shown above, and an appropriate aryl amine, e.g., 3.6 as shown above. Appropriate acetophenone halides and appropriate aryl amines are commercially available or prepared by methods known to one skilled in the art. The coupling reaction is carried out in the presence of an appropriate catalyst, e.g., palladium (II) acetate, an appropriate ligand, e.g., 2,2′-bis(diphenylphosphino)-1,1′-binaphthyl (BINAP), and an appropriate base, e.g., cesium carbonate, in an appropriate solvent, e.g., toluene. Compounds of type 3.8 can be prepared by a cyclization reaction of an appropriate acetophenone, e.g., 3.7 as shown above. The cyclization reaction is carried out in the presence of an appropriate activating agent, e.g., carbon tetrabromide, an appropriate thiourea, e.g., thiourea as shown above, and an appropriate base, e.g., triethylamine (TEA), in an appropriate solvent, e.g., acetonitrile. Compounds of type 3.9 can be prepared by a coupling reaction between an appropriate amine, e.g., 3.8 as shown above, and an appropriate carboxylic acid, e.g., 1.11 as shown above. Appropriate carboxylic acids are commercially available or prepared by methods known to one skilled in the art. The coupling reaction is carried out in the presence of an appropriate coupling agent, e.g., 1-[bis(dimethylamino)methylene]-1H-1,2,3-triazolo[4,5-b]pyridinium 3-oxid hexafluorophosphate (HATU), and an appropriate base, e.g., diisopropylethylamine (DIPEA), in an appropriate solvent, e.g., dimethylformamide, at an appropriate temperature, e.g., 100° C. As can be appreciated by one skilled in the art, the above reaction provides an example of a generalized approach wherein compounds similar in structure to the specific reactants above (compounds similar to compounds of type 1.2b, 1.5, 3.1, 3.2, and 3.3), can be substituted in the reaction to provide substituted biphenyl thiazole derivatives similar to Formula 3.4.

### E. METHODS OF USING THE COMPOUNDS

The compounds and pharmaceutical compositions of the invention are useful in treating or controlling disorders associated with a viral infection, in particular, CHIKV, VEEV, DENV, WNV, influenza, and ZIKV.

Examples of viral infections for which the compounds and compositions can be useful in treating, include, but are not limited to, human immunodeficiency virus (HIV), human papillomavirus (HPV), influenza, chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, chikungunya, Venezuelan equine encephalitis, dengue, influenza, and zika.

To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a viral infection, such as CHIKV, VEEV, DENV, WNV, influenza, and ZIKV.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a viral infection, such as CHIKV, VEEV, DENV, WNV, influenza, and ZIKV.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

1. Treatment Methods

The compounds disclosed herein are useful for treating or controlling disorders associated with a viral infection, in particular, CHIKV, VEEV, DENV, WNV, influenza, and ZIKV. Thus, provided is a method comprising administering a therapeutically effective amount of a composition comprising a disclosed compound to a subject. In a further aspect, the method can be a method for treating a viral infection.

a. Treating a Viral Infection

In one aspect, disclosed are methods of treating a viral infection in a subject having the viral infection, the method comprising the step of administering to the subject a therapeutically effective amount of at least one disclosed compound, or a pharmaceutically acceptable salt thereof.

In one aspect, disclosed are methods for the treatment of a viral infection in a subject having the viral infection, the method comprising the step of administering to the subject a therapeutically effective amount of at least one compound having a structure represented by a formula:

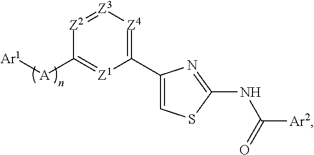

wherein n is selected from 0 and 1; wherein A, when present, is selected from O, NH, N(C1-C4 alkyl), and N(Ar³); wherein Ar³, when present, is phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of Z¹, Z², Z³, and Z⁴ is independently selected from N and CR¹⁰, provided that at least two of Z¹, Z², Z³, and Z⁴ are CR¹⁰; wherein each occurrence of R¹⁰, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², —SO₂R²³, and Cyl; wherein each occurrence of R²⁰, R²¹, R²², and R²³, when present, is independently selected from hydrogen and C1-C4 alkyl; wherein Cy¹, when present, is selected from 5-membered heterocycle and 6-membered heterocycle, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl; wherein R¹ is selected from hydrogen, —CN, —CO₂H, C1-C4 alkyl, and C1-C4 alkylamino; wherein R² is selected from hydrogen, C1-C4 alkyl, Ar⁴, and —CH₂Ar⁴; wherein Ar⁴, when present, is phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and either: (a) wherein Ar¹ is selected from pyridinyl, piperazinyl, and 6-membered aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴; wherein each occurrence of R¹, R¹², R¹³, and R¹⁴, when present, is independently selected from hydrogen and C1-C4 alkyl; and wherein Ar² is selected from 5-membered heterocycle, 6-membered heterocycle, 5-membered heteroaryl, and 6-membered heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸; and wherein each occurrence of R¹⁵, R¹⁶, R¹⁷, and R¹⁸, when present, is independently selected from hydrogen and C1-C4 alkyl; or (b) wherein Ar¹ is a structure represented by a formula:

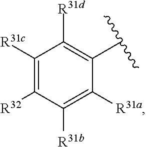

wherein each of R^{31a}, R^{31b}, R^{31c}, and R^{31a} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴; wherein R³² is selected from —CN and —CH₂NH₂; and wherein Ar² is selected from 5-membered heterocycle, 6-membered heterocycle, 5-membered heteroaryl, 6-membered heteroaryl, 6-membered aryl, and bicyclo[1.1.1]pentanyl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸, or a pharmaceutically acceptable salt thereof.

Examples of viral infections include, but are not limited to, human immunodeficiency virus (HIV), human papillomavirus (HPV), influenza, chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, chikungunya, Venezuelan equine encephalitis, dengue, influenza, and zika.

In a further aspect, the subject has been diagnosed with a need for treatment of the disorder prior to the administering step.

In a further aspect, the subject is a mammal. In a still further aspect, the mammal is a human.

In a further aspect, the method further comprises the step of identifying a subject in need of treatment of the viral infection.

In a further aspect, the effective amount is a therapeutically effective amount. In a still further aspect, the effective amount is a prophylactically effective amount.

In a further aspect, the disorder is associated with a viral infection. In a still further aspect, the viral infection is selected from human immunodeficiency virus (HIV), human papillomavirus (HPV), influenza, chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, chikungunya, Venezuelan equine encephalitis, dengue, influenza, and zika. In yet a further aspect, the viral infection is selected from chikungunya, Venezuelan equine encephalitis, dengue, influenza, and zika. In an even further aspect, the viral infection is selected from CHIKV, VEEV, DENV, WNV, influenza, and ZIKV. In a still further aspect, the viral infection is CHIKV.

In a further aspect, the method further comprises the step of administering a therapeutically effective amount of at least one antiviral agent. In a still further aspect, the at least one agent is selected from acemannan, acyclovir, acyclovir sodium, adamantanamine, adefovir, adenine arabinoside, alovudine, alvircept sudotox, amantadine hydrochloride, aranotin, arildone, atevirdine mesylate, avridine, cidofovir, cipamfylline, cytarabine hydrochloride, BMS 806, C31G, carrageenan, cellulose sulfate, cyclodextrins, dapivirine, delavirdine mesylate, desciclovir, dextrin 2-sulfate, didanosine, disoxaril, dolutegravir, edoxudine, enviradene, envirozime, etravirine, famciclovir, famotine hydrochloride, fiacitabine, fialuridine, fosarilate, foscarnet sodium, fosfonet sodium, FTC, ganciclovir, ganciclovir sodium, GSK 1265744, 9-2-hydroxy-ethoxy methylguanine, ibalizumab, idoxuridine, interferon, 5-iodo-2′-deoxyuridine, IQP-0528, kethoxal, lamivudine, lobucavir, maraviroc, memotine pirodavir, penciclovir, raltegravir, ribavirin, rimantadine hydrochloride, rilpivirine (TMC-278), saquinavir mesylate, SCH-C, SCH-D, somantadine hydrochloride, sorivudine, statolon, stavudine, T20, tilorone hydrochloride, TMC120, TMC125, trifluridine, trifluorothymidine, tenofovir, tenofovir alefenamide, tenofovir disoproxyl fumarate, prodrugs of tenofovir, UC-781, UK-427, UK-857, valacyclovir, valacyclovir hydrochloride, vidarabine, vidarabine phosphate, vidarabine sodium phosphate, viroxime, zalcitabene, zidovudine, and zinviroxime.

In a further aspect, the at least one compound and the at least one agent are administered sequentially. In a still further aspect, the at least one compound and the at least one agent are administered simultaneously.

In a further aspect, the at least one compound and the at least one agent are co-formulated. In a still further aspect, the at least one compound and the at least one agent are co-packaged.

2. Methods of Inhibiting a Viral Infection in a Mammal

In one aspect, disclosed are methods of inhibiting a viral infection in a mammal, the method comprising the step of administering to the mammal a therapeutically effective amount of at least one disclosed compound, or a pharmaceutically acceptable salt thereof.

Thus, in one aspect, disclosed are methods of inhibiting a viral infection in a mammal, the method comprising the step of administering to the mammal a therapeutically effective amount of at least one compound having a structure represented by a formula:

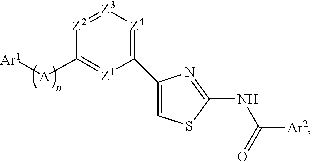

wherein n is selected from 0 and 1; wherein A, when present, is selected from O, NH, N(C1-C4 alkyl), and N(Ar³); wherein Ar³, when present, is phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of Z¹, Z², Z³, and Z⁴ is independently selected from N and CR¹⁰, provided that at least two of Z², Z², Z³, and Z⁴ are CR¹⁰; wherein each occurrence of R¹⁰, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², —SO₂R²³, and Cy¹; wherein each occurrence of R²⁰, R²¹, R²², and R²³, when present, is independently selected from hydrogen and C1-C4 alkyl; wherein Cy¹, when present, is selected from 5-membered heterocycle and 6-membered heterocycle, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl; wherein R¹ is selected from hydrogen, —CN, —CO₂H, C1-C4 alkyl, and C1-C4 alkylamino; wherein R² is selected from hydrogen, C1-C4 alkyl, Ar⁴, and —CH₂Ar⁴; wherein Ar⁴, when present, is phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and either: (a) wherein Ar¹ is selected from pyridinyl, piperazinyl, and 6-membered aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴; wherein each occurrence of R¹¹, R¹², R¹³, and R¹⁴, when present, is independently selected from hydrogen and C1-C4 alkyl; and wherein Ar² is selected from 5-membered heterocycle, 6-membered heterocycle, 5-membered heteroaryl, and 6-membered heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸; and wherein each occurrence of R¹⁵, R¹⁶, R¹⁷, and R¹⁸, when present, is independently selected from hydrogen and C1-C4 alkyl; or (b) wherein Ar¹ is a structure represented by a formula:

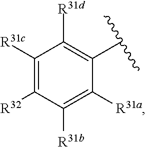

wherein each of R^{31a}, R^{31b}, R^{31c}, and R^{31d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴; wherein R³² is selected from —CN and —CH₂NH₂; and wherein Ar² is selected from 5-membered heterocycle, 6-membered heterocycle, 5-membered heteroaryl, 6-membered heteroaryl, 6-membered aryl, and bicyclo[1.1.1]pentanyl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸, or a pharmaceutically acceptable salt thereof.

In a further aspect, the compound exhibits inhibition of a viral infection. In a still further aspect, the compound exhibits a decrease in a viral infection. In yet a further aspect, the viral infection is CHIKV.

In a further aspect, the compound exhibits inhibition of CHIKV activity with an EC₉₀ of less than about 30 μM. In a still further aspect, the compound exhibits inhibition of CHIKV activity with an EC₉₀ of less than about 25 μM. In yet a further aspect, the compound exhibits inhibition of CHIKV activity with an EC₉₀ of less than about 20 μM. In an even further aspect, the compound exhibits inhibition of CHIKV activity with an EC₉₀ of less than about 15 μM. In a still further aspect, the compound exhibits inhibition of CHIKV activity with an EC₉₀ of less than about 10 μM. In yet a further aspect, the compound exhibits inhibition of CHIKV activity with an EC₉₀ of less than about 5 μM. In an even further aspect, the compound exhibits inhibition of CHIKV activity with an EC₉₀ of less than about 1 μM. In a still further aspect, the compound exhibits inhibition of CHIKV activity with an EC₉₀ of less than about 0.5 μM.

In a further aspect, the subject is a mammal. In a still further aspect, the subject is a human.

In a further aspect, the subject has been diagnosed with a need for treatment of the disorder prior to the administering step. In a still further aspect, the method further comprises the step of identifying a subject in need of treatment of the disorder.

3. Methods of Inhibiting a Viral Infection in at Least One Cell

In one aspect, disclosed are methods for inhibiting a viral infection in at least one cell, the method comprising the step of contacting the at least one cell with an effective amount of at least one disclosed compound, or a pharmaceutically acceptable salt thereof.

Thus, in one aspect, disclosed are methods for inhibiting a viral infection in at least one cell, the method comprising the step of contacting the at least one cell with an effective amount of at least one compound having a structure represented by a formula:

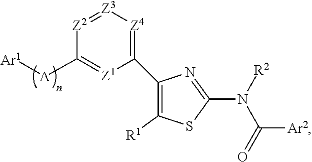

wherein n is selected from 0 and 1; wherein A, when present, is selected from O, NH, N(C1-C4 alkyl), and N(Ar³); wherein Ar³, when present, is phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of Z¹, Z², Z³, and Z⁴ is independently selected from N and CR¹⁰, provided that at least two of Z¹, Z², Z³, and Z⁴ are CR¹⁰; wherein each occurrence of R¹⁰, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², —SO₂R²³, and Cy¹; wherein each occurrence of R²⁰, R²¹, R²², and R²³, when present, is independently selected from hydrogen and C1-C4 alkyl; wherein Cy¹, when present, is selected from 5-membered heterocycle and 6-membered heterocycle, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl; wherein R¹ is selected from hydrogen, —CN, —CO₂H, C1-C4 alkyl, and C1-C4 alkylamino; wherein R² is selected from hydrogen, C1-C4 alkyl, Ar⁴, and —CH₂Ar⁴; wherein Ar⁴, when present, is phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and either: (a) wherein Ar¹ is selected from pyridinyl, piperazinyl, and 6-membered aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴; wherein each occurrence of R¹, R¹², R¹³, and R¹⁴, when present, is independently selected from hydrogen and C1-C4 alkyl; and wherein Ar² is selected from 5-membered heterocycle, 6-membered heterocycle, 5-membered heteroaryl, and 6-membered heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸; and wherein each occurrence of R, R¹⁶, R¹⁷, and R¹⁸, when present, is independently selected from hydrogen and C1-C4 alkyl; or (b) wherein Ar¹ is a structure represented by a formula:

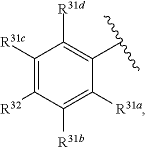

wherein each of R^{31a}, R^{31b}, R^{31c}, and R^{31d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴; wherein R³² is selected from —CN and —CH₂NH₂; and wherein Ar² is selected from 5-membered heterocycle, 6-membered heterocycle, 5-membered heteroaryl, 6-membered heteroaryl, 6-membered aryl, and bicyclo[1.1.1]pentanyl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸, or a pharmaceutically acceptable salt thereof.

In a further aspect, the cell is mammalian. In a still further aspect, the cell is human. In yet a further aspect, the cell has been isolated from a mammal prior to the contacting step.

In a further aspect, contacting is via administration to a mammal.

4. Use of Compounds

In one aspect, the invention relates to the use of a disclosed compound or a product of a disclosed method. In a further aspect, a use relates to the manufacture of a medicament for the treatment of a viral infection in a subject.

Also provided are the uses of the disclosed compounds and products. In one aspect, the invention relates to use of at least one disclosed compound; or a pharmaceutically acceptable salt, hydrate, solvate, or polymorph thereof. In a further aspect, the compound used is a product of a disclosed method of making.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to a treatment of a viral infection in a subject. Also disclosed is the use of a compound for antagonism of a viral infection. In one aspect, the use is characterized in that the subject is a human. In one aspect, the use is characterized in that the disorder is a viral infection.

In a further aspect, the use relates to the manufacture of a medicament for the treatment of a viral infection in a subject.

In a further aspect, the use relates to antagonism of a viral infection in a subject. In a further aspect, the use relates to modulating viral activity in a subject. In a still further aspect, the use relates to modulating viral activity in a cell. In yet a further aspect, the subject is a human.

It is understood that the disclosed uses can be employed in connection with the disclosed compounds, products of disclosed methods of making, methods, compositions, and kits. In a further aspect, the invention relates to the use of a disclosed compound or a disclosed product in the manufacture of a medicament for the treatment of a viral infection in a mammal. In a further aspect, the viral infection is selected from CHIKV, VEEV, DENV, WNV, influenza, and ZIKV.

5. Manufacture of a Medicament

In one aspect, the invention relates to a method for the manufacture of a medicament for treating a viral infection in a subject having the viral infection, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in the inhibition of a viral infection. The dose administered to an animal, particularly a human, in the context of the present invention should be sufficient to affect a therapeutic response in the animal over a reasonable time frame. One skilled in the art will recognize that dosage will depend upon a variety of factors including the condition of the animal and the body weight of the animal.

The total amount of the compound of the present disclosure administered in a typical treatment is preferably between about 10 mg/kg and about 1000 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

6. Kits

In one aspect, disclosed are kits comprising at least one disclosed compound and one or more of: (a) at least one antiviral agent; (b) a instructions for administering the at least one compound in connection with treating a viral infection; (c) instructions for administering the at least one compound in connection with reducing the risk of viral infection; and (d) instructions for treating a viral infection.

In a further aspect, the viral infection is selected from human immunodeficiency virus (HIV), human papillomavirus (HPV), influenza, chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, chikungunya, Venezuelan equine encephalitis, dengue, influenza, and zika. In a still further aspect, the viral infection is selected from chikungunya, Venezuelan equine encephalitis, dengue, influenza, West Nile, and zika. In yet a further aspect, the viral infection is CHIKV.

In a still further aspect, the antiviral agent is selected from selected from acemannan, acyclovir, acyclovir sodium, adamantanamine, adefovir, adenine arabinoside, alovudine, alvircept sudotox, amantadine hydrochloride, aranotin, arildone, atevirdine mesylate, avridine, cidofovir, cipamfylline, cytarabine hydrochloride, BMS 806, C31G, carrageenan, cellulose sulfate, cyclodextrins, dapivirine, delavirdine mesylate, desciclovir, dextrin 2-sulfate, didanosine, disoxaril, dolutegravir, edoxudine, enviradene, envirozime, etravirine, famciclovir, famotine hydrochloride, fiacitabine, fialuridine, fosarilate, foscamet sodium, fosfonet sodium, FTC, ganciclovir, ganciclovir sodium, GSK 1265744, 9-2-hydroxy-ethoxy methylguanine, ibalizumab, idoxuridine, interferon, 5-iodo-2′-deoxyuridine, IQP-0528, kethoxal, lamivudine, lobucavir, maraviroc, memotine pirodavir, penciclovir, raltegravir, ribavirin, rimantadine hydrochloride, rilpivirine (TMC-278), saquinavir mesylate, SCH-C, SCH-D, somantadine hydrochloride, sorivudine, statolon, stavudine, T20, tilorone hydrochloride, TMC120, TMC125, trifluridine, trifluorothymidine, tenofovir, tenofovir alefenamide, tenofovir disoproxyl fumarate, prodrugs of tenofovir, UC-781, UK-427, UK-857, valacyclovir, valacyclovir hydrochloride, vidarabine, vidarabine phosphate, vidarabine sodium phosphate, viroxime, zalcitabene, zidovudine, and zinviroxime.

In a further aspect, the at least one compound and the at least one agent are co-formulated. In a further aspect, the at least one compound and the at least one agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Chemistry Experimentals

### a. General Experimental

The reactions were performed under a dry argon atmosphere and reaction temperatures were measured externally. Anhydrous solvents over molecular sieves were purchased from Aldrich and used as such in reactions. Microwave (MW) reactions were performed in CEM Discover Labmate System with Intelligent Technology for Focused™ Microwave Synthesizer (Explorer 48) or Biotage Initiator+eqipped with Robot Eight microwave system. The reactions were monitored by thin-layer chromatography (TLC) on pre-coated silica gel (60F₂₅₄) aluminium plates (0.25 mm) from E. Merck and visualized using UV light (254 nm). Purification of compounds was performed on an Isco Teledyne Combiflash Rf200. Universal RediSep solid sample loading pre-packed cartridges (5.0 g silica) were used to absorb crude product and purified on 12 g silica RediSep Rf Gold Silica (20-40 μm spherical silica) columns using appropriate solvent gradients. Pure samples were dried overnight under high vacuum before analyses. The high resolution electrospray ionization mass spectral data (HR-ESIMS) were obtained on an Agilent LC-MSTOF. ¹H NMR spectra were recorded at 400 MHz on Agilent/Varian MR-400 spectrometer in CDC₃, CD₃OD, or DMSO-d₆ as solvents. The chemical shifts (δ) are in ppm downfield from standard tetramethylsilane (TMS). HPLC of final compounds were run on an Agilent 1100 LC equipped with a diode array UV detector and were monitored at 254 nm using the following using Sunfire C18 column (5 μm, 4.6×150 mm) using H₂O—CH₃CN (both containing 0.1% formic acid) 5-95% in 20 min with flow rate 1.0 mL/min.

### b. Synthesis of Compounds 1-4, 6-19 and 23

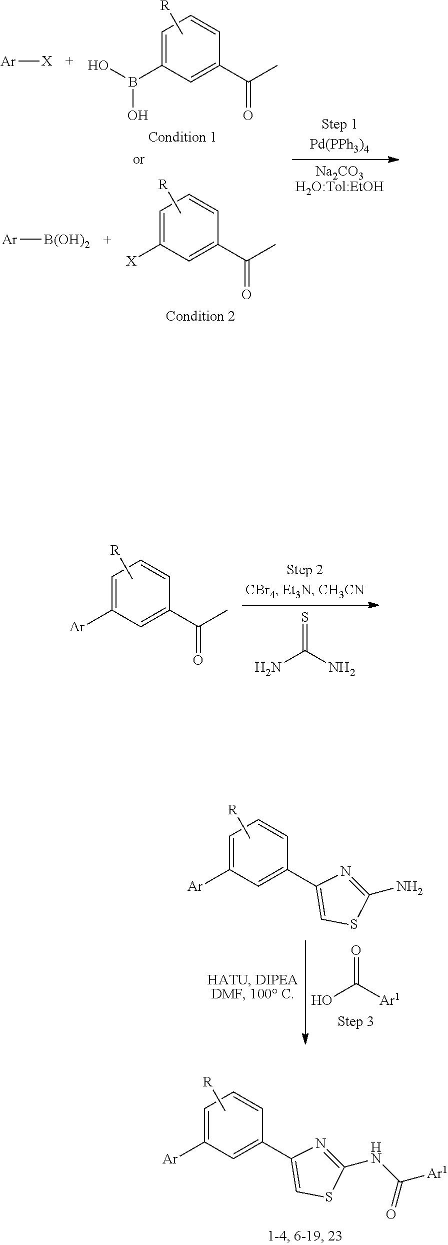

i. Step 1

General Procedure for conditions 1: Corresponding aryl halide (0.8 mmol) was added to a solution of corresponding acetophenone boronic acid (1 mmol), tetrakis(triphenylphosphine) palladium (0.02 mmol) and sodium carbonate (6 mmol) in anhydrous toluene (3 mL), H₂O (3 mL) and EtOH (1 mL) at room temperature. The reaction mixture was then stirred at 90° C. for 18 hrs. After cooling to rt, the reaction mixture was treated with H₂O (5 mL) and extracted with EtOAc (3×5 mL). The organic layer was collected, dried over Na₂SO₄, filtered and evaporated to dryness under reduced pressure to give a residue, which was purified on ISCO to yield the product.

General Procedure for conditions 2: Corresponding acetophenone halide (0.8 mmol) was added to a solution of corresponding aryl boronic acid (1 mmol), tetrakis(triphenylphosphine) palladium (0.02 mmol) and sodium carbonate (6 mmol) in anhydrous toluene (3 mL), H₂O (3 mL) and EtOH (1 mL) at room temperature. The reaction mixture was then stirred at 90° C. for 18 hrs. After cooling to rt, the reaction mixture was treated with H₂O (5 mL) and extracted with EtOAc (3×5 mL). The organic layer was collected, dried over Na₂SO₄, filtered and evaporated to dryness under reduced pressure to give a residue, which was purified on ISCO to yield the product.

### (i) 1-[3-(2-Pyridyl)phenyl]ethanone

Condition 1. Purification on pre-packed Silica gel column on ISCO using 0-70% EtOAc in Hexanes (30 min). Yield: 1.51 g (63%). ¹H NMR (CDCl₃) δ 8.72 (dt, J=4.8, 1.5 Hz, 1H), 8.58 (td, J=1.8, 0.6 Hz, 1H), 8.21 (ddd, J=7.8, 1.9, 1.1 Hz, 1H), 8.01 (ddd, J=7.8, 1.8, 1.1 Hz, 1H), 7.80 (d, J=1.4 Hz, 1H), 7.79 (t, J=1.4 Hz, 1H), 7.58 (td, J=7.8, 0.6 Hz, 1H), 7.31-7.26 (m, 1H), 2.68 (s, 3H). ESIMS: m/z 198.1 [M+H]+.

### (ii) 1-[3-(4-Pyridyl)phenyl]ethanone

Condition 1. Purification on pre-packed Silica gel column on ISCO using 0-70% EtOAc in Hexanes (30 min). Yield: 0.77 g (64%). ¹H NMR (CDCl₃) δ 8.69 (ddd, J=4.4, 2.8, 1.7 Hz, 2H), 8.22 (dtd, J=2.5, 1.9, 0.6 Hz, 1H), 8.01 (dddd, J=7.7, 2.6, 1.7, 1.1 Hz, 1H), 7.83 (dddd, J=7.7, 2.6, 1.9, 1.1 Hz, 1H), 7.65-7.56 (m, 1H), 7.53 (ddd, J=4.4, 2.7, 1.7 Hz, 2H), 2.67 (s, 3H). ESIMS: m/z 198.1 [M+H]+.

### (iii) 4-(3-Acetylphenyl)benzonitrile

Condition 1. Purification on pre-packed Silica gel column on ISCO using 0-70% EtOAc in Hexanes (30 min). Yield: 1.01 g (75%). ¹H NMR (CDCl₃) δ 8.21-8.16 (m, 1H), 8.00 (ddd, J=7.7, 1.7, 1.1 Hz, 1H), 7.79 (ddd, J=7.7, 1.9, 1.1 Hz, 1H), 7.78-7.77 (m, 1H), 7.76-7.75 (m, 1H), 7.74-7.73 (m, 1H), 7.72-7.71 (m, 1H), 7.60 (td, J=7.7, 0.6 Hz, 1H), 2.67 (s, 3H). ESIMS: m/z 222.1 [M+H]+.

### (iv) 4-(5-Acetyl-3-pyridyl)benzonitrile

Condition 2. Purification on pre-packed Silica gel column on ISCO using 0-70% EtOAc in Hexanes (30 min). Yield: 0.84 g (56%). ¹H NMR (CDCl₃) δ 9.20 (d, J=2.3 Hz, 1H), 9.03 (d, J=2.6 Hz, 1H), 8.43 (t, J=2.4 Hz, 1H), 7.87-7.78 (m, 2H), 7.78-7.70 (m, 2H), 2.72 (s, 3H). ESIMS: m/z 223.1 [M+H]+.

### (v) 4-(6-Acetyl-2-pyridyl)benzonitrile

Condition 2. Purification on pre-packed Silica gel column on ISCO using 0-70% EtOAc in Hexanes (30 min). Yield: 1.023 g (68%). ¹H NMR (CDCl₃) δ 8.25-8.20 (m, 2H), 8.08-8.04 (m, 1H), 7.98-7.97 (m, 1H), 7.96 (d, J=1.5 Hz, 1H), 7.83-7.78 (m, 2H), 2.82 (s, 3H). ESIMS: m/z 223.1 [M+H]+.

### (vi) 6-(3-Acetylphenyl)pyridine-3-carbonitrile

Condition 1. Purification on pre-packed Silica gel column on ISCO using 0-70% EtOAc in Hexanes (30 min). Yield: 0.761 g (56%). ¹H NMR (CDCl₃) δ 8.97 (dd, J=2.2, 1.0 Hz, 1H), 8.64 (td, J=1.8, 0.5 Hz, 1H), 8.28 (ddd, J=7.8, 1.9, 1.1 Hz, 1H), 8.14-8.01 (m, 2H), 7.99-7.86 (m, 1H), 7.69-7.56 (m, 1H), 2.70 (s, 3H). ESIMS: m/z 223.1 [M+H]+.

### (vii) 1-[3-(5-Fluoro-2-pyridyl)phenyl]ethanone

Condition 1. Purification on pre-packed Silica gel column on ISCO using 0-70% EtOAc in Hexanes (30 min). Yield: 0.869 g (66%). ¹H NMR (CDCl₃) δ 8.57 (d, J=2.9 Hz, 1H), 8.54 (t, J=1.8 Hz, 1H), 8.17 (ddd, J=7.7, 1.9, 1.1 Hz, 1H), 8.01 (ddd, J=7.8, 1.8, 1.1 Hz, 1H), 7.80 (ddd, J=8.8, 4.2, 0.7 Hz, 1H), 7.58 (td, J=7.8, 0.6 Hz, 1H), 7.51 (ddd, J=8.8, 8.0, 2.9 Hz, 1H), 2.69 (s, 3H). ESIMS: m/z 216.1 [M+H].

### (viii) 1-[3-(6-Fluoro-3-pyridyl)phenyl]ethanone

Condition 1. Purification on pre-packed Silica gel column on ISCO using 0-70% EtOAc in Hexanes (30 min). Yield: 0.896 g (68%). ¹H NMR (CDCl₃) δ 8.47-8.41 (m, 1H), 8.12 (td, J=1.9, 0.6 Hz, 1H), 8.06-7.94 (m, 2H), 7.78-7.70 (m, 1H), 7.62-7.54 (m, 1H), 7.07-7.00 (m, 1H), 2.66 (s, 3H). ESIMS: m/z 216.1 [M+H]+.

### (ix) 4-(3-Acetyl-4-fluoro-phenyl)benzonitrile

Condition 2. Purification on pre-packed Silica gel column on ISCO using 0-70% EtOAc in Hexanes (30 min). Yield: 1.01 g (62%). ¹H NMR (CDCl₃) δ 8.11 (ddd, J=6.9, 2.6, 0.4 Hz, 1H), 7.79-7.72 (m, 3H), 7.70-7.65 (m, 2H), 7.30-7.25 (m, 1H), 2.70 (d, J=5.0 Hz, 3H). ESIMS: m/z 240.1 [M+H]+.

### (x) 4-(3-Acetyl-4-methoxy-phenyl)benzonitrile

Condition 2. Purification on pre-packed Silica gel column on ISCO using 0-70% EtOAc in Hexanes (30 min). Yield: 0.791 g (46%). ¹H NMR (CDCl₃) δ 8.00 (d, J=2.5 Hz, 1H), 7.82-7.56 (m, 5H), 7.09 (d, J=8.7 Hz, 1H), 3.99 (s, 3H), 2.66 (s, 3H). ESIMS: m/z 252.1 [M+H]+.

### (xi) Methyl-4-(3-acetylphenyl)benzoate

Condition 1. Purification on pre-packed Silica gel column on ISCO using 0-70% EtOAc in Hexanes (30 min). Yield: 0.871 g (56%). ¹H NMR (CDCl₃) 8.21 (td, J=1.8, 0.5 Hz, 1H), 8.16-8.14 (m, 1H), 8.13 (dq, J=1.6, 0.8 Hz, 1H), 7.98 (dddd, J=7.8, 1.7, 1.1, 0.5 Hz, 1H), 7.83 (dddd, J=7.8, 1.8, 1.1, 0.5 Hz, 1H), 7.72-7.70 (m, 1H), 7.69 (dd, J=1.3, 0.7 Hz, 1H), 7.58 (tt, J=7.7, 0.6 Hz, 1H), 3.96 (s, 3H), 2.67 (s, 3H). ESIMS: m/z 255.1 [M+H]+.

### (xii) 3′-Acetyl-5′-fluoro-[1,1′-biphenyl]-4-carbonitrile

Condition 1. Purification on pre-packed Silica gel column on ISCO using 0-70% EtOAc in Hexanes (30 min). Yield: 1.1 g (68%). ¹H NMR (CDCl₃) δ 7.97 (t, J=1.5 Hz, 1H), 7.83-7.75 (m, 2H), 7.75-7.63 (m, 3H), 7.50 (dt, J=9.0, 2.0 Hz, 1H), 2.66 (s, 3H). ESIMS: m/z 240.1

### II. Step 2

General Procedure: Carbon tetrabromide (1 mmol) was added to a solution of the product from Step-1 (1 mmol), thiourea (1 mmol) and triethylamine (1 mmol) in anhydrous CH₃CN (10 mL). The reaction mixture was then stirred at rt for 18 hrs. The reaction was treated with H₂O (10 mL) and extracted with EtOAc (3×10 mL). The organic layer was collected, dried over anhydrous Na₂SO₄, filtered and evaporated to dryness under reduced pressure to give a residue, which was purified on ISCO to yield the product.

### (i) 4-([1,1′-Biphenyl]-3-yl)thiazol-2-amine

Purification on pre-packed Silica gel column on ISCO using 0-70% EtOAc in Hexanes (30 min). Yield: 0.143 g (37%). ¹H NMR (CDCl₃): δ 8.08-7.92 (m, 1H), 7.75 (dt, J=7.7, 1.5 Hz, 1H), 7.69-7.62 (m, 2H), 7.53 (ddd, J=7.7, 1.8, 1.2 Hz, 1H), 7.45 (td, J=7.5, 2.7 Hz, 3H), 7.40-7.31 (m, 1H), 6.76 (d, J=1.7 Hz, 1H), 5.39 (s, 2H). ESIMS: m/z 253.1 [M+H]+.

### (ii) 4-[3-(2-pyridyl)phenyl]thiazol-2-amine

Purification on pre-packed Silica gel column on ISCO using 0-70% EtOAc in Hexanes (30 min). Yield: 0.74 g (38%). ¹H NMR (CDCl₃) δ 8.72 (ddd, J=4.8, 1.8, 1.0 Hz, 1H), 8.43 (td, J=1.8, 0.6 Hz, 1H), 7.92 (ddd, J=7.8, 1.9, 1.1 Hz, 1H), 7.85-7.80 (m, 1H), 7.80-7.77 (m, 1H), 7.77-7.73 (m, 1H), 7.49 (td, J=7.7, 0.5 Hz, 1H), 7.26-7.22 (m, 1H), 6.84 (s, 1H), 5.26 (s, 2H). ESIMS: m/z 254.1 [M+H]+.

### (iii) 4-[3-(4-Pyridyl)phenyl]thiazol-2-amine

Purification on pre-packed Silica gel column on ISCO using 0-70% EtOAc in Hexanes (30 min). Yield: 0.27 g (27%). ¹H NMR (CDCl₃) δ 8.67 (d, J=6.2 Hz, 2H), 8.09 (t, J=1.9 Hz, 1H), 7.84 (dt, J=7.7, 1.5 Hz, 1H), 7.62-7.53 (m, 3H), 7.50 (t, J=7.7 Hz, 1H), 6.82 (s, 1H), 5.01 (s, 2H). ESIMS: m/z 254.1 [M+H].

### (iv) 4-[3-(2-Aminothiazol-4-yl)phenyl]benzonitrile

Purification on pre-packed Silica gel column on ISCO using 0-70% EtOAc in Hexanes (30 min). Yield: 0.27 g (21%). ¹H NMR (CDCl₃) δ 8.04 (td, J=1.4, 0.5 Hz, 1H), 7.81 (dt, J=6.8, 1.9 Hz, 1H), 7.77-7.68 (m, 4H), 7.55-7.43 (m, 2H), 6.81 (s, 1H), 5.05 (s, 2H). ESIMS: m/z 278.0 [M+H]+.

### (v) 4-[5-(2-Aminothiazol-4-yl)-3-pyridyl]benzonitrile

Purification on pre-packed Silica gel column on ISCO using 0-70% EtOAc in Hexanes (30 min). Yield: 0.159 g (15%). ¹H NMR (CDCl₃) δ 9.04 (d, J=2.1 Hz, 1H), 8.74 (d, J=2.3 Hz, 1H), 8.30 (t, J=2.2 Hz, 1H), 7.81-7.72 (m, 4H), 6.92 (s, 1H), 5.04 (s, 2H). ESIMS: m/z 279.0 [M+H]+.

### (vi) 4-[6-(2-Aminothiazol-4-yl)-2-pyridyl]benzonitrile

Purification on pre-packed Silica gel column on ISCO using 0-70% EtOAc in Hexanes (30 min). Yield: 0.23 g (18%). ¹H NMR (CDCl₃) δ 8.23-8.18 (m, 2H), 7.93 (dd, J=7.8, 1.0 Hz, 1H), 7.84 (t, J=7.8 Hz, 1H), 7.80-7.75 (m, 2H), 7.67 (dd, J=7.8, 1.0 Hz, 1H), 7.52 (s, 1H), 4.97 (s, 2H). ESIMS: m/z 279.0 [M+H]+.

### (vii) 6-[3-(2-Aminothiazol-4-yl)phenyl]pyridine-3-carbonitrile

Purification on pre-packed Silica gel column on ISCO using 0-70% EtOAc in Hexanes (30 min). Yield: 70 mg (28%). ¹H NMR (CDCl₃) δ 8.96 (dd, J=2.2, 0.9 Hz, 1H), 8.48 (t, J=1.8 Hz, 1H), 8.01 (dd, J=8.3, 2.2 Hz, 1H), 7.96 (ddd, J=7.8, 1.9, 1.0 Hz, 1H), 7.92 (dd, J=8.3, 0.9 Hz, 1H), 7.88 (ddd, J=7.8, 1.8, 1.0 Hz, 1H), 7.56-7.48 (m, 1H), 6.84 (s, 1H), 5.10 (s, 2H). ESIMS: m/z 279.1 [M+H]+.

### (viii) 4-[3-(5-Fluoro-2-pyridyl)phenyl]thiazol-2-amine

Purification on pre-packed Silica gel column on ISCO using 0-70% EtOAc in Hexanes (30 min). Yield: 0.374 g (34%). ¹H NMR (CDCl₃) δ 8.56 (dt, J=2.9, 0.5 Hz, 1H), 8.38 (t, J=1.8 Hz, 1H), 7.86 (ddd, J=7.8, 1.9, 1.1 Hz, 1H), 7.83-7.81 (m, 1H), 7.81-7.77 (m, 1H), 7.51-7.48 (m, 1H), 7.48-7.45 (m, 1H), 6.83 (s, 1H), 5.23 (s, 2H). ESIMS: m/z 272.1 [M+H]⁺.

### (ix) 4-[3-(6-Fluoro-3-pyridyl)phenyl]thiazol-2-amine

Purification on pre-packed Silica gel column on ISCO using 0-70% EtOAc in Hexanes (30 min). Yield: 0.297 g (27%). ¹H NMR (CDCl₃) δ 8.47 (ddd, J=2.6, 1.1, 0.6 Hz, 1H), 8.02 (ddd, J=8.5, 7.6, 2.6 Hz, 1H), 7.98 (td, J=1.7, 0.7 Hz, 1H), 7.79 (dt, J=7.1, 1.8 Hz, 1H), 7.51-7.43 (m, 2H), 7.01 (ddd, J=8.4, 3.0, 0.7 Hz, 1H), 6.81 (s, 1H), 5.03 (s, 2H). ESIMS: m/z 272.0 [M+H]⁺.

### (x) 4-[3-(2-Aminothiazol-4-yl)-4-fluoro-phenyl]benzonitrile

Purification on pre-packed Silica gel column on ISCO using 0-70% EtOAc in Hexanes (30 min). Yield: 0.201 g (23%). ¹H NMR (CDCl₃) δ 8.32 (dd, J=7.3, 2.5 Hz, 1H), 7.74-7.71 (m, 4H), 7.46 (ddd, J=8.5, 4.6, 2.5 Hz, 1H), 7.21 (dd, J=11.2, 8.5 Hz, 1H), 7.12 (d, J=2.3 Hz, 1H), 4.96 (s, 2H). ESIMS: m/z 296.0 [M+H]⁺.

### (xi) 4-[3-(2-Aminothiazol-4-yl)-4-methoxy-phenyl]benzonitrile

Purification on pre-packed Silica gel column on ISCO using 0-70% EtOAc in Hexanes (30 min). Yield: 0.168 g (23%). ¹H NMR (CDCl₃) δ 8.39 (d, J=2.5 Hz, 1H), 7.76-7.66 (m, 4H), 7.50 (dd, J=8.5, 2.5 Hz, 1H), 7.29 (s, 1H), 7.06 (d, J=8.6 Hz, 1H), 4.91 (s, 2H), 4.00 (s, 3H). ESIMS: m/z 308.1 [M+H]⁺.

### (xii) Methyl-4-[3-(2-aminothiazol-4-yl)phenyl]benzoate

Purification on pre-packed Silica gel column on ISCO using 0-70% EtOAc in Hexanes (30 min). Yield: 0.165 g (34%). ¹H NMR (CDCl₃) δ 8.15-8.08 (m, 2H), 8.06 (td, J=1.8, 0.6 Hz, 1H), 7.80 (ddd, J=7.6, 1.7, 1.2 Hz, 1H), 7.76-7.67 (m, 2H), 7.55 (ddd, J=7.7, 1.9, 1.2 Hz, 1H), 7.47 (td, J=7.7, 0.6 Hz, 1H), 6.82 (s, 1H), 4.95 (s, 2H), 3.95 (s, 3H). ESIMS: m/z 311.1 [M+H]⁺.

### (xiii) 3′-(2-Aminothiazol-4-yl)-5′-fluoro-[1,1′-biphenyl]-4-carbonitrile

Purification on pre-packed Silica gel column on ISCO using 0-70% EtOAc in Hexanes (30 min). Yield: 220 mg (36%): ¹H NMR (CDCl₃) δ 7.82 (t, J=1.5 Hz, 1H), 7.77-7.69 (m, 4H), 7.55-7.48 (m, 1H), 7.26-7.14 (m, 1H), 6.83 (s, 1H). ESIMS: m/z 296.1 [M+H]⁺.

### iii. Step 3

General Procedure: HATU (1.5 mmol) and DIPEA (3 mmol) were added to a solution of corresponding carboxylic acid (1 mmol) in anhydrous DMF (16 mL). After stirring for 10 min at rt, the product from Step-2 (1 mmol) was added. The reaction mixture was then stirred at 100° C. for 18 hrs. After cooling to rt, the reaction mixture was treated with ice cold H₂O (100 mL) and extracted with EtOAc (3×50 mL). The organic layer was collected, dried over anhydrous Na₂SO₄, filtered and evaporated to dryness under reduced pressure to give a residue, which was purified on ISCO to yield the product.

### (i) N-(4-([1,1′-Biphenyl]-3-yl)thiazol-2-yl)-2-(methylthio)nicotinamide (1)

Purification on pre-packed Silica gel column on ISCO using 0-70% EtOAc in Hexanes (30 min). Yield: 49 mg (31%). ¹H NMR (CDCl₃) δ 11.62 (s, 1H), 8.37 (dd, J=4.8, 1.8 Hz, 1H), 7.88 (t, J=1.6 Hz, 1H), 7.70 (dd, J=7.7, 1.8 Hz, 1H), 7.65 (dt, J=7.7, 1.4 Hz, 1H), 7.63-7.58 (m, 2H), 7.51-7.44 (m, 3H), 7.42-7.36 (m, 2H), 7.23 (s, 1H), 6.81 (dd, J=7.7, 4.8 Hz, 1H), 2.51 (s, 3H). HR-ESIMS: m/z 404.0885 [M+H]⁺ calcd. for C₂₂H₁₈N₃OS₂, found 404.0885. HPLC purity: 99% (Retention Time=17.8 min).

### (ii) 2-(Methylthio)-N-(4-(3-(pyridin-2-yl)phenyl)thiazol-2-yl)nicotinamide (2)

Purification on pre-packed Silica gel column on ISCO using 0-70% EtOAc in Hexanes (30 min). Yield: 80 mg (45%). ¹H NMR (CDCl₃) δ 8.73 (dt, J=4.8, 1.5 Hz, 1H), 8.50 (dd, J=4.8, 1.8 Hz, 1H), 8.47-8.41 (m, 1H), 7.92 (ddd, J=7.7, 3.0, 1.4 Hz, 2H), 7.83 (ddd, J=7.7, 1.8, 1.1 Hz, 1H), 7.80-7.75 (m, 2H), 7.49 (t, J=7.8 Hz, 1H), 7.32 (s, 1H), 7.29-7.26 (m, 1H), 7.01 (dd, J=7.7, 4.8 Hz, 1H), 2.60 (s, 3H). HR-ESIMS: m/z 405.0838 [M+H]⁺ calcd. for C₂₁H₁₇N₄OS₂, found 405.0834. HPLC purity: 98% (Retention Time=16.0 min).

### (iii) 2-(Methylthio)-N-(4-(3-(pyridin-4-yl)phenyl)thiazol-2-yl)nicotinamide (3)

Purification on pre-packed Silica gel column on ISCO using 0-70% EtOAc in Hexanes (30 min). Yield: 35 mg (22%). ¹H NMR (DMSO-d₆) δ 12.89 (s, 1H), 8.69-8.64 (m, 2H), 8.62 (dd, J=4.8, 1.7 Hz, 1H), 8.33 (t, J=1.7 Hz, 1H), 8.13 (dd, J=7.7, 1.8 Hz, 1H), 8.03 (ddd, J=7.7, 1.8, 1.1 Hz, 1H), 7.92 (s, 1H), 7.79-7.73 (m, 3H), 7.60 (t, J=7.8 Hz, 1H), 7.26 (dd, J=7.7, 4.9 Hz, 1H), 2.47 (s, 3H). HR-ESIMS: m/z 405.0838 [M+H]⁺ calcd. for C₂₁H₁₇N₄OS₂, found 405.0841. HPLC purity: 95% (Retention Time=10.6 min).

### (iv) N-(4-(4′-Cyano-[1,1′-biphenyl]-3-yl)thiazol-2-yl)-2-(methylthio)nicotinamide (4)

Purification on pre-packed Silica gel column on ISCO using 0-70% EtOAc in Hexanes (30 min). Yield: 76 mg (49%). ¹H NMR (CDCl₃) δ 12.09 (s, 1H), 8.28 (dd, J=5.0, 2.0 Hz, 1H), 7.85-7.79 (m, 1H), 7.75-7.68 (m, 2H), 7.68-7.63 (m, 3H), 7.63-7.56 (m, 2H), 7.46-7.34 (m, 2H), 6.71 (dd, J=7.9, 4.9 Hz, 1H), 2.43 (s, 3H). HR-ESIMS: m/z 429.0838 [M+H]⁺ calcd. for C₂₃H₁₇N₄OS₂, found 429.0835. HPLC purity: 97% (Retention Time=17.0 min).

### (v) 5-(Tert-butyl)-N-(4-(4′-cyano-[1,1′-biphenyl]-3-yl)thiazol-2-yl)pyrimidine-2-carboxamide (8)

Purification on pre-packed Silica gel column on ISCO 0-70% EtOAc in Hexanes (30 min). Yield: 47 mg (40%). ¹H NMR (CDCl₃) δ 11.12 (s, 1H), 8.96 (s, 2H), 8.15 (dt, J=1.6, 1.0 Hz, 1H), 7.91 (ddd, J=6.3, 2.5, 1.7 Hz, 1H), 7.85-7.67 (m, 4H), 7.62-7.49 (m, 2H), 7.32 (d, J=0.8 Hz, 1H), 1.55 (s, 3H), 1.46 (s, 6H). HR-ESIMS: m/z 440.1540 [M+H]⁺ calcd. for C₂₅H₂₂N₅OS, found 440.1537. HPLC purity: 98% (Retention Time=17.6 min).

### (vi) 4-(Tert-butyl)-N-(4-(4′-cyano-[1,1′-biphenyl]-3-yl)thiazol-2-yl)benzamide (9)

Purification on pre-packed Silica gel column on ISCO using 0-70% EtOAc in Hexanes (30 min). Yield: 63 mg (40%). ¹H NMR (CDCl₃) δ 9.67 (s, 1H), 8.10 (td, J=1.8, 0.8 Hz, 1H), 7.93-7.83 (m, 3H), 7.79-7.71 (m, 4H), 7.57-7.49 (m, 4H), 7.28 (s, 1H), 1.36 (s, 9H). HR-ESIMS: m/z 438.1635 [M+H]⁺ calcd. for C₂₇H₂₄N₃OS, found 438.1628. HPLC purity: 98% (Retention Time=19.8 min).

### (vii) N-(4-(5-(4-Cyanophenyl)pyridin-3-yl)thiazol-2-yl)-2-(methylthio)nicotinamide (12)

Purification on pre-packed Silica gel column on ISCO using 0-70% EtOAc in Hexanes (30 min). Yield: 25 mg (32%). ¹H NMR (DMSO-d₆) δ 9.15 (d, J=2.0 Hz, 1H), 8.78 (d, J=2.3 Hz, 1H), 8.53 (t, J=2.2 Hz, 1H), 8.41 (dd, J=4.7, 1.9 Hz, 1H), 8.37 (dd, J=7.6, 1.9 Hz, 1H), 8.07-7.92 (m, 4H), 7.47 (s, 1H), 7.10 (dd, J=7.6, 4.7 Hz, 1H), 2.33 (s, 3H). HR-ESIMS: m/z 430.0791 [M+H]⁺ calcd. for C₂₂H₁₆N₅OS₂, found 430.0784. HPLC purity: 100% (Retention Time=11.9 min).

### (viii) N-(4-(6-(4-Cyanophenyl)pyridin-2-yl)thiazol-2-yl)-2-(methylthio)nicotinamide (13)

Purification on pre-packed Silica gel column on ISCO 0-70% EtOAc in Hexanes (30 min). Yield: 35 mg (23%). ¹H NMR (CDCl₃) δ 8.40 (dd, J=4.8, 1.8 Hz, 1H), 8.23-8.16 (m, 2H), 7.92 (s, 1H), 7.84-7.80 (m, 2H), 7.79-7.75 (m, 3H), 7.65 (dd, J=7.5, 1.3 Hz, 1H), 6.92 (dd, J=7.7, 4.8 Hz, 1H), 2.57 (s, 3H). HR-ESIMS: m/z 430.0791 [M+H]⁺ calcd. for C₂₂H₁₆N₅OS₂, found 430.0780. HPLC purity: 95% (Retention Time=16.4 min).

### (ix) N-(4-(3-(5-Cyanopyridin-2-yl)phenyl)thiazol-2-yl)-2-(methylthio)nicotinamide (14)

Purification on pre-packed Silica gel column on ISCO using 0-70% EtOAc in Hexanes (30 min). Yield: 32 mg (30%). ¹H NMR (CDCl₃) δ 10.49 (s, 1H), 8.98 (dd, J=2.2, 0.9 Hz, 1H), 8.57-8.52 (m, 2H), 8.03 (dd, J=8.3, 2.2 Hz, 1H), 7.98 (d, J=1.8 Hz, 1H), 7.96 (d, J=1.7 Hz, 1H), 7.95-7.90 (m, 2H), 7.54 (t, J=7.8 Hz, 1H), 7.34 (d, J=0.4 Hz, 1H), 7.07 (dd, J=7.7, 4.8 Hz, 1H), 2.62 (s, 3H). HR-ESIMS: m/z 430.0791 [M+H]⁺ calcd. for C₂₂H₁₆N₅OS₂, found 430.0786. HPLC purity: 95% (Retention Time=15.6 min).

### (x) N-(4-(3-(5-Fluoropyridin-2-yl)phenyl)thiazol-2-yl)-2-(methylthio)nicotinamide (15)

Purification on pre-packed Silica gel column on ISCO using 0-70% EtOAc in Hexanes (30 min). Yield: 52 mg (33%). ¹H NMR (CDCl₃) δ 10.72 (s, 1H), 8.57 (d, J=3.1 Hz, 1H), 8.50 (dd, J=4.8, 1.8 Hz, 1H), 8.39 (d, J=1.7 Hz, 1H), 7.91 (dd, J=7.7, 1.7 Hz, 1H), 7.86 (ddd, J=7.7, 1.9, 1.1 Hz, 1H), 7.84-7.80 (m, 1H), 7.78 (dd, J=8.8, 4.3 Hz, 1H), 7.52-7.45 (m, 2H), 7.31 (s, 1H), 7.04-6.96 (m, 1H), 2.59 (s, 3H). HR-ESIMS: m/z 423.0744 [M+H]⁺ calcd. for C₂₁H₁₆FN₄OS₂, found 423.0740. HPLC purity: 98% (Retention Time=15.7 min).

### (xi) N-(4-(3-(6-Fluoropyridin-3-yl)phenyl)thiazol-2-yl)-2-(methylthio)nicotinamide (16)

Purification on pre-packed Silica gel column on ISCO using 0-70% EtOAc in Hexanes (30 min). Yield: 46 mg (30%). ¹H NMR (CDCl₃) δ 10.62 (s, 1H), 8.53 (dd, J=4.8, 1.8 Hz, 1H), 8.48 (ddd, J=2.7, 1.1, 0.6 Hz, 1H), 8.02 (ddd, J=8.4, 7.6, 2.6 Hz, 1H), 7.98 (td, J=1.7, 0.7 Hz, 1H), 7.94 (dd, J=7.7, 1.8 Hz, 1H), 7.80 (dt, J=6.9, 1.9 Hz, 1H), 7.52-7.44 (m, 2H), 7.28 (s, 1H), 7.08-7.00 (m, 2H), 2.62 (s, 3H). HR-ESIMS: m/z 423.0744 [M+H]⁺ calcd. for C₂₁H₁₆FN₄OS₂, found 423.0739. HPLC purity: 97% (Retention Time=15.8 min).

### (xii) N-(4-(4′-Cyano-4-fluoro-[1,1′-biphenyl]-3-yl)thiazol-2-yl)-2-(methylthio)nicotinamide (17)

Purification on pre-packed Silica gel column on ISCO using 0-70% EtOAc in Hexanes (30 min). Yield: 30 mg (20%). ¹H NMR (CDCl₃) δ 10.06 (s, 1H), 8.62 (dd, J=4.8, 1.8 Hz, 1H), 8.35 (dd, J=7.2, 2.4 Hz, 1H), 8.12-8.00 (m, 1H), 7.82-7.66 (m, 4H), 7.58 (d, J=2.3 Hz, 1H), 7.50 (ddd, J=8.4, 4.6, 2.5 Hz, 1H), 7.29-7.23 (m, 1H), 7.20-7.10 (m, 1H), 2.67 (s, 3H). HR-ESIMS: m/z 447.0744 [M+H]⁺ calcd. for C₂₃H₁₆FN₄OS₂, found 447.0732. HPLC purity: 95% (Retention Time=17.4 min).

### (xiii) N-(4-(4′-Cyano-4-methoxy-[1,1′-biphenyl]-3-yl)thiazol-2-yl)-2-(methylthio)nicotinamide (18)

Purification on pre-packed Silica gel column on ISCO using 0-70% EtOAc in Hexanes (30 min). Yield: 15 mg (20%). ¹H NMR (CDCl₃) δ 8.48 (ddd, J=4.8, 1.9, 0.8 Hz, 1H), 8.26 (dd, J=2.5, 0.9 Hz, 1H), 7.85 (dd, J=7.7, 1.7 Hz, 1H), 7.72-7.62 (m, 5H), 7.49 (dd, J=8.5, 2.5 Hz, 1H), 7.04 (d, J=8.6 Hz, 1H), 7.02-6.92 (m, 1H), 4.00 (s, 3H), 2.55 (s, 3H). HR-ESIMS: m/z 459.0944 [M+H]⁺ calcd. for C₂₄H₁₉N₄O₂S₂, found 459.0939. HPLC purity: 95% (Retention Time=17.2 min).

### (xiv) Methyl 3′-(2-(2-(methylthio)nicotinamido)thiazol-4-yl)-[1,1′-biphenyl]-4-carboxylate (23)

Purification on pre-packed Silica gel column on ISCO using 0-70% EtOAc in Hexanes (30 min). Yield: 64 mg (26%). ¹H NMR (CDCl₃) δ 8.46 (dd, J=4.8, 1.8 Hz, 1H), 8.19-8.06 (m, 2H), 8.01 (t, J=1.8 Hz, 1H), 7.85 (dd, J=7.7, 1.8 Hz, 1H), 7.76 (ddt, J=7.7, 1.9, 1.0 Hz, 1H), 7.71-7.68 (m, 2H), 7.54 (ddd, J=7.7, 1.8, 1.2 Hz, 1H), 7.46 (td, J=7.7, 0.5 Hz, 1H), 7.27 (s, 1H), 6.96 (dd, J=7.7, 4.8 Hz, 1H), 3.95 (s, 3H), 2.57 (s, 3H). HR-ESIMS: m/z 462.0941 [M+H]⁺ cald. for C₂₄H₂₀N₃O₃S₂, found 462.0934. HPLC purity: 99% (Retention Time=17.5 min).

### (xv) N-(4-(4′-Cyano-[1,1′-biphenyl]-3-yl)thiazol-2-yl)-2-((difluoromethyl)thio)nicotinamide (6)

Purification on pre-packed Silica gel column on ISCO using 0-60% EtOAc in Hexanes (45 min). Yield: 11 mg (57%). ¹H NMR (CDCl₃) δ 10.96 (s, 1H), 8.48 (dd, J=4.8, 1.7 Hz, 1H), 7.96-7.91 (m, 1H), 7.86 (dd, J=7.8, 1.7 Hz, 1H), 7.80 (s, 1H), 7.78-7.64 (m, 5H), 7.54-7.46 (m, 2H), 7.30 (s, 1H), 7.08 (dd, J=7.8, 4.8 Hz, 1H). HR-ESIMS: m/z 465.0577 [M+H]⁺ calcd. for C₂₃H₁₅F₂N₄OS₂, found 465.0641. HPLC purity: 98% (Retention Time=17.7 min).

### (xvi) N-(4-(4′-Cyano-[1,1′-biphenyl]-3-yl)thiazol-2-yl)-2-(trifluoromethyl)nicotinamide (7)

Purification on pre-packed Silica gel column on ISCO using 0-50% EtOAc in Hexanes (30 min). Yield: 29 mg (80%). ¹H NMR (400 MHz, CDCl₃) δ 8.79 (dd, J=4.8, 1.1 Hz, 1H), 8.00-7.89 (m, 2H), 7.79 (d, J=7.4 Hz, 1H), 7.73 (d, J=1.4 Hz, 4H), 7.59-7.46 (m, 3H), 7.28 (s, 1H). HR-ESIMS: m/z 451.0762 [M+H]⁺ calcd. for C₂₃H₁₄F₃N₄OS, found 451.0724. HPLC purity: 97% (Retention Time=15.7 min).

### (xvii) N-(4-(4′-Cyano-[1,1′-biphenyl]-3-yl)thiazol-2-yl)-3,5-difluoro-4-methoxybenzamide (10)

Purification on pre-packed Silica gel column on ISCO using 0-70% EtOAc in Hexanes (30 min). Yield: 45 mg (33%). ¹H NMR (400 MHz, CDCl₃) δ 10.73 (s, 1H), 7.98 (d, J=1.7 Hz, 1H), 7.90-7.58 (m, 5H), 7.54-7.41 (m, 2H), 7.38 (d, J=9.0 Hz, 2H), 7.31 (s, 1H), 4.04 (t, J=1.6 Hz, 3H). HR-ESIMS: m/z 448.0853 [M+H]⁺ calcd. for C₂₄H₁₆F₃N₃O₂S, found 448.0913. HPLC purity: 93% (Retention Time=18.2 min).

### (xviii) N-(4-(4′-Cyano-[1,1′-biphenyl]-3-yl)thiazol-2-yl)piperidine-4-carboxamide (11)

This compound was made following general procedure for Step-3 followed by deprotection of Boc group. To a DCM solution of tert-Butyl 4-[[4-[3-(4-cyanophenyl)phenyl]thiazol-2-yl]carbamoyl]piperidine-1-carboxylate (90 mg, 0.18 mmol) at 0° C., trifluoroacetic acid (0.56 mL, 7.37 mmol) was added and the reaction was stirred at rt for 1 hour. After completion of reaction it was neutralized with aq. NaHCO₃ and extracted with EtOAc (2×50 mL). The combined organic layers was dried over anhydrous Na₂SO₄, concentrated under reduced pressure followed by purification on preparative TLC plate using DCM-MeOH (9:1) produced the product. Yield: 49 mg (68%). ¹H NMR (CDCl₃) δ 8.01 (t, J=1.5 Hz, 1H), 7.78 (dt, J=7.3, 1.6 Hz, 1H), 7.68 (s, 4H), 7.52-7.41 (m, 2H), 7.30-7.22 (m, 1H), 7.18 (s, 1H), 3.36 (d, J=12.7 Hz, 2H), 3.28 (s, 1H), 3.02 (d, J=10.4 Hz, 2H), 2.74 (s, 1H), 2.16-2.00 (m, 2H), 2.01-1.91 (m, 2H). HR-ESIMS: m/z 389.1358 [M+H]⁺ calcd. for C₂₂H₂₁N₄OS, found 389.1436. HPLC purity: 99% (Retention Time=6.5 min).

### (xix) N-(4-(4′-Cyano-5-fluoro-[1,1′-biphenyl]-3-yl)thiazol-2-yl)-2-(methylthio)nicotinamide (19)

Purification on pre-packed Silica gel column on ISCO using 0-10% MeOH in CH₂Cl₂ (30 min). Yield: 13 mg (10%). ¹H NMR (400 MHz, CDCl₃) δ 10.25 (s, 1H), 8.59 (dd, J=4.8, 1.8 Hz, 1H), 8.03 (dd, J=7.7, 1.8 Hz, 1H), 7.85 (t, J=1.5 Hz, 1H), 7.81-7.68 (m, 4H), 7.57 (ddd, J=9.5, 2.4, 1.5 Hz, 1H), 7.31 (s, 1H), 7.25-7.20 (m, 1H), 7.13 (dd, J=7.7, 4.8 Hz, 1H), 2.66 (s, 3H). HR-ESIMS: m/z 447.0671 [M+H]⁺ calcd. for C₂₃H₁₆FN₄OS₂, found 447.0734. HPLC purity: 95% (Retention Time=14.2 min).

### c. 3′-(2-(2-(Methylthio)nicotinamido)thiazol-4-yl)-[1,1′-biphenyl]-4-carboxylic acid (24)

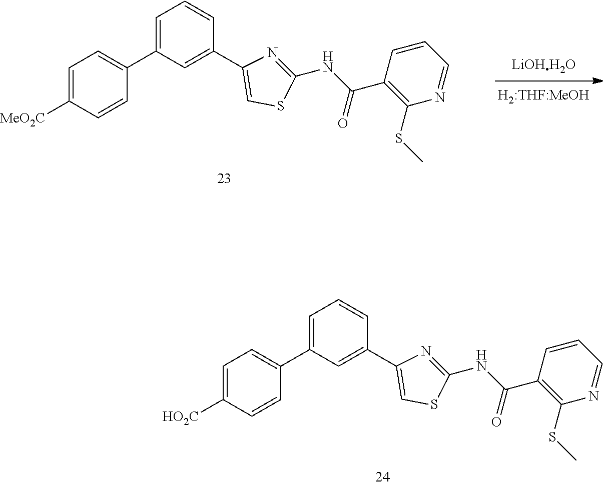

Lithium hydroxide monohydrate (9.1 mg, 0.22 mmol) was added to a solution of methyl 3′-(2-(2-(methylthio)nicotinamido)thiazol-4-yl)-[1,1′-biphenyl]-4-carboxylate (23) (50 mg, 0.11 mmol) in MeOH (2 mL), THF (1 mL) and H₂O (0.5 mL). The reaction mixture was then stirred at rt for 18 hrs. The solution was concentrated to dryness under reduced pressure, and the residue was neutralized with TN HCl. A precipitate was formed immediately which was collected by filtration, and washed with H₂O to yield the product as a yellow solid. Yield: 28 mg (58%). ¹H NMR (DMSO-d₆) δ 12.88 (s, 1H), 8.62 (dd, J=4.8, 1.7 Hz, 1H), 8.29 (t, J=1.8 Hz, 1H), 8.12 (dd, J=7.7, 1.8 Hz, 1H), 8.08-8.02 (m, 2H), 7.98 (dt, J=7.8, 1.4 Hz, 1H), 7.90 (s, 1H), 7.88-7.82 (m, 2H), 7.70 (ddd, J=7.7, 2.0, 1.1 Hz, 1H), 7.57 (t, J=7.8 Hz, 1H), 7.26 (dd, J=7.7, 4.8 Hz, 1H), 2.49 (s, 3H). ESIMS: m/z 448.0 [M+H]⁺. HPLC purity: 97% (Retention Time=2.1 min).

### d. Synthesis of 2-methylsulfanyl-N-[4-(3-phenoxyphenyl)thiazol-2-yl]pyridine-3-carboxamide (36)

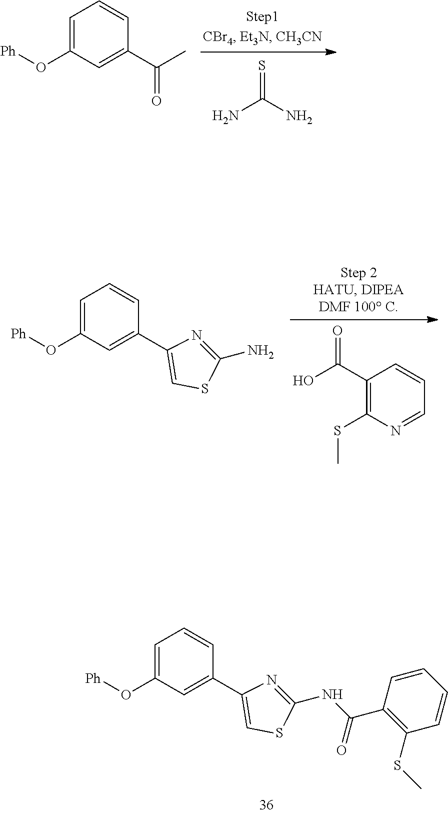

Step 1. Synthesis of 4-(3-phenoxyphenyl)thiazol-2-amine: Carbon tetrabromide (1.56 g, 4.71 mmol) was added to a solution of 1-(3-phenoxyphenyl)ethanone (1.0 g, 4.71 mmol), thiourea (0.36 g, 4.71 mmol) and triethylamine (0.66 mL, 4.71 mmol) in anhydrous CH₃CN (43 mL). The reaction mixture was then stirred at rt for 48 hrs. The reaction was treated with H₂O (10 mL) and extracted with EtOAc (3×10 mL). The organic layer was collected, dried over anhydrous Na₂SO₄, filtered and evaporated to dryness under reduced pressure to give a residue, which was purified on ISCO using 0-50% EtOAc in Hexanes (20 min) to yield the product as an orange solid. Yield: 0.507 g (40%). ¹H NMR (CDCl₃) δ 7.51 (ddd, J=7.7, 1.6, 1.0 Hz, 1H), 7.42 (dd, J=2.5, 1.6 Hz, 1H), 7.38-7.30 (m, 3H), 7.11 (ddt, J=8.5, 7.2, 1.1 Hz, 1H), 7.07-7.00 (m, 2H), 6.94 (ddd, J=8.1, 2.5, 1.0 Hz, 1H), 6.70 (s, 1H), 5.10 (s, 2H). ESIMS: m/z 269.1 [M+H].

Step 2. Synthesis of 2-(methylthio)-N-(4-(3-phenoxyphenyl)thiazol-2-yl)nicotinamide (36): HATU (106 mg, 0.28 mmol) and DIPEA (0.10 mL, 0.56 mmol) were added to a solution of 2-methylsulfanylpyridine-3-carboxylic acid (32 mg, 0.19 mmol) in anhydrous DMF (3 mL). After stirring for 10 min at rt, 4-(3-phenoxyphenyl)thiazol-2-amine (50 mg, 0.19 mmol) was added. The reaction mixture was then stirred at 100° C. for 18 hrs. After cooling to rt, the reaction mixture was treated with ice cold H₂O (50 mL) and extracted with EtOAc (3×20 mL). The organic layer was collected, dried over anhydrous Na₂SO₄, filtered and evaporated to dryness under reduced pressure to give a residue, which was purified on ISCO using 0-70% EtOAc in Hexanes (30 min) to yield the product as an orange solid. Yield: 20 mg (26%). ¹H NMR (CDCl₃) δ 11.52 (s, 1H), 8.43 (dd, J=4.8, 1.8 Hz, 1H), 7.67 (dd, J=7.7, 1.8 Hz, 1H), 7.44-7.24 (m, 6H), 7.12-7.01 (m, 3H), 6.90 (ddd, J=8.1, 2.5, 1.0 Hz, 1H), 6.84 (dd, J=7.6, 4.8 Hz, 1H), 2.52 (s, 3H). HR-ESIMS: m/z 420.0835 [M+H]⁺ calcd. for C₂₂H₁₅N₃O₂S₂, found 420.0825. HPLC purity: 96% (Retention Time=17.5 min).

### e. Synthesis of Compounds 5, 20-22, 25, 27, 28

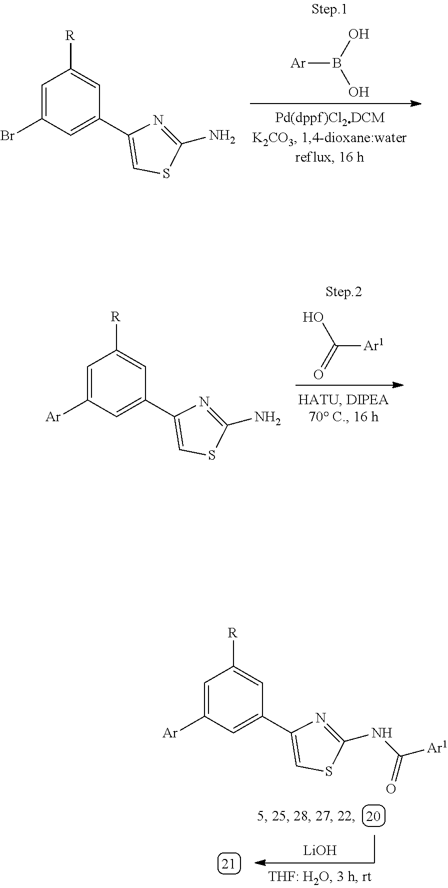

### i. Step 1: General Procedure

4-(3-Bromophenyl)-1,3-thiazol-2-amine (1 mmol) was dissolved in water and 1,4-dioxane (1:1) and to this solution, aryl boronic acid (1.5 mmol) was added followed by 1,1′-Bis(Diphenylphosphino)ferrocenepalladium (II) dichloride (0.20 mmol) and anhydrous potassium carbonate (3 mmol). The reaction mixture was stirred under nitrogen for 16 hrs and filtered through short pad of celite. The filtrate was diluted with water (50 mL) and extracted with EtOAc (2×150 mL). The organic layer was dried over anhydrous Na₂SO₄ and concentrated under reduced pressure followed by purification on pre-packed Silica gel column on ISCO purification system to yield the product.

### (i) 3′-(2-Aminothiazol-4-yl)-[1,1′-biphenyl]-3-carbonitrile

Purification on pre-packed Silica gel column on ISCO using 0-60% EtOAc in Hexanes (45 min). Yield: 210 mg (77%). ¹H NMR (CDCl₃) δ 7.92-7.86 (m, 2H), 7.83 (ddd, J=7.8, 1.9, 1.3 Hz, 1H), 7.70 (ddd, J=5.1, 3.6, 1.7 Hz, 1H), 7.59 (dt, J=7.7, 1.4 Hz, 1H), 7.51 (td, J=7.8, 0.6 Hz, 1H), 7.47-7.38 (m, 2H), 6.71 (s, 1H). ESIMS: m/z 278.1 [M+H]⁺.

### (ii) 3′-(2-Aminothiazol-4-yl)-[1,1′-biphenyl]-4-carboxamide

Purification on pre-packed Silica gel column on ISCO using 0-20% MeOH in CH₂Cl₂ (30 min). Yield: 130 mg (56%). ¹H NMR (CDCl₃) δ 8.01 (s, 1H), 7.88 (d, J=8.2 Hz, 2H), 7.71 (d, J=8.3 Hz, 2H), 7.52 (s, 1H), 7.46 (d, J=7.7 Hz, 2H), 6.76 (s, 1H). ESIMS: m/z 296.1 [M+H]⁺.

### (iii) 4-(3-(2-Methoxypyrimidin-5-yl)phenyl)thiazol-2-amine

Purification on pre-packed Silica gel column on ISCO using 0-50% EtOAc in Hexanes (45 min). Yield: 18 mg (81%). ¹H NMR (CDCl₃) δ 8.72 (d, J=37.8 Hz, 2H), 8.04-7.88 (m, 1H), 7.80 (dt, J=7.6, 1.5 Hz, 1H), 7.58-7.33 (m, 2H), 6.81 (s, 1H), 4.08 (d, J=2.4 Hz, 3H). ESIMS: m/z 285.1 [M+H]⁺.

### (iv) Tert-butyl ((3′-(2-aminothiazol-4-yl)-[1,1′-biphenyl]-4-yl)methyl)carbamate

Purification on pre-packed Silica gel column on ISCO using 0-50% EtOAc in Hexanes (30 min). Yield: 193 mg (52%). ¹H NMR (CDCl₃) δ 8.00 (t, J=1.8 Hz, 1H), 7.79-7.70 (m, 1H), 7.61 (d, J=8.3 Hz, 2H), 7.49 (s, 1H), 7.45 (d, J=7.6 Hz, 1H), 7.36 (d, J=8.2 Hz, 2H), 6.79 (s, 1H), 4.94 (s, 2H), 1.48 (s, 9H). ESIMS: m/z 382.1 [M+H]⁺.

### (v) 3′-(2-Aminothiazol-4-yl)-3-methoxy-[1,1′-biphenyl]-4-carbonitrile

Purification on pre-packed Silica gel column on ISCO using 0-60% EtOAc in Hexanes (45 min). Yield: 310 mg (86%). ¹H NMR (CDCl₃) δ 8.07-7.93 (m, 1H), 7.81 (ddd, J=5.4, 3.2, 1.7 Hz, 1H), 7.62 (d, J=8.0 Hz, 1H), 7.56-7.41 (m, 2H), 7.26-7.24 (m, 1H), 7.18 (d, J=1.4 Hz, 1H), 6.82 (s, 1H), 4.01 (s, 3H). ESIMS: m/z 308.1 [M+H]⁺.

### (vi) Methyl 5-(2-aminothiazol-4-yl)-4′-cyano-[1,1′-biphenyl]-3-carboxylate

Purification on pre-packed Silica gel column on ISCO using 0-30% EtOAc in Hexanes (45 min). Yield: 66 mg (62%). ¹H NMR (CDCl₃): δ 8.35 (t, J=1.6 Hz, 1H), 8.27-7.89 (m, 2H), 7.91-7.51 (m, 4H), 6.88-6.71 (m, 1H), 4.06-3.68 (m, 3H). ESIMS: m/z 336.1 [M+H]⁺.

### ii. Step 2: General Synthetic Procedure

To a solution of carboxylic acid (1 mmol) in anhydrous DMF (5 mL) under nitrogen was added HATU (1.5 mmol) followed by DIPEA (3 mmol) at room temperature and the reaction mixture was stirred for about 10 min. Corresponding substituted 2-aminothiazoles (from Step-1) was added to the reaction mixture and heated at 70° C. for 16 hrs. The mixture was allowed to cool to room temperature, diluted with water (100 mL) and extracted with EtOAc (2×150 mL). The organic layer was washed with brine, dried over anhydrous Na₂SO₄ and solvent was removed under reduced pressure. The crude was purified using pre-packed Silica gel column on ISCO purification system to yield product.

### (i) N-(4-(3′-Cyano-[1,1′-biphenyl]-3-yl)thiazol-2-yl)-2-(methylthio)nicotinamide (5)

Purification on pre-packed Silica gel column on ISCO using 0-50% EtOAc in Hexanes (30 min). Yield: 71 mg (55%). ¹H NMR (CDCl₃) δ 11.39 (s, 1H), 8.40 (dd, J=4.8, 1.8 Hz, 1H), 7.92-7.85 (m, 2H), 7.82 (ddd, J=7.8, 1.8, 1.3 Hz, 1H), 7.76 (dd, J=7.7, 1.8 Hz, 1H), 7.71 (ddd, J=6.0, 2.6, 1.8 Hz, 1H), 7.65 (dt, J=7.7, 1.4 Hz, 1H), 7.56 (td, J=7.8, 0.5 Hz, 1H), 7.48-7.39 (m, 2H), 7.27 (s, 1H), 6.88 (dd, J=7.7, 4.8 Hz, 1H), 2.53 (s, 3H). HR-ESIMS: m/z 429.0766 [M+H]⁺ calcd. for C₂₃H₁₇N₄OS₂, found 429.0829. HPLC purity: 97.8% (Retention Time=16.6 min).

### (ii) N-(4-(4′-Carbamoyl-[1,1′-biphenyl]-3-yl)thiazol-2-yl)-2-(methylthio)nicotinamide (25)

Purification on pre-packed Silica gel column on ISCO using 0-10% MeOH in CH₂Cl₂ (45 min). Yield: 45 mg (54%). ¹H NMR (CDCl₃) δ 12.89 (s, 1H), 8.61 (dd, J=4.9, 1.7 Hz, 1H), 8.27 (d, J=1.6 Hz, 1H), 8.13 (dd, J=7.8, 1.6 Hz, 1H), 8.03 (s, 1H), 7.97 (dd, J=10.3, 8.2 Hz, 3H), 7.88 (s, 1H), 7.80 (d, J=8.4 Hz, 2H), 7.68 (d, J=8.0 Hz, 1H), 7.55 (t, J=7.8 Hz, 1H), 7.40 (s, 1H), 7.25 (dd, J=7.7, 4.9 Hz, 1H), 2.46 (s, 3H). HR-ESIMS: m/z 447.0871 [M+H]⁺ calcd. for C₂₃H₁₉N₄O₂S₂, found 447.0940. HPLC purity: 96% (Retention Time=11.9 min).

### (iii) N-(4-(3-(2-Methoxypyrimidin-5-yl)phenyl)thiazol-2-yl)-2-(methylthio)nicotinamide (28)

Purification on pre-packed Silica gel column on ISCO using 0-10% MeOH in CH₂Cl₂ (30 min). Yield: 65 mg (48%). ¹H NMR (CDCl₃) δ 8.71 (s, 2H), 8.51 (dd, J=4.8, 1.8 Hz, 1H), 8.02-7.84 (m, 2H), 7.77 (d, J=7.7 Hz, 1H), 7.46 (s, 2H), 7.23 (s, 1H), 7.07 (dd, J=7.7, 4.8 Hz, 1H), 3.99 (s, 3H), 2.52 (s, 3H). HR-ESIMS: m/z 436.0824 [M+H]⁺ calcd. for C₂₁H₁₈N₅O₂S₂, found 436.0902. HPLC purity: 96% (Retention Time=14.5 min).

### (iv) N-(4-(4′-Cyano-3′-methoxy-[1,1′-biphenyl]-3-yl)thiazol-2-yl)-2-(methylthio)nicotinamide

Purification on pre-packed Silica gel column on ISCO using 0-50% EtOAc in Hexanes (30 min). Yield: 98 mg (72%). ¹H NMR (CDCl₃) δ 12.89 (s, 1H), 8.62 (d, J=4.2 Hz, 1H), 8.27 (s, 1H), 8.12 (d, J=7.5 Hz, 1H), 8.01 (d, J=7.9 Hz, 1H), 7.92 (s, 1H), 7.83 (d, J=8.0 Hz, 1H), 7.74 (d, J=7.5 Hz, 1H), 7.58 (t, J=7.6 Hz, 1H), 7.51 (s, 1H), 7.43 (d, J=7.8 Hz, 1H), 7.26 (dd, J=7.4, 5.0 Hz, 1H), 4.03 (s, 3H), 2.47 (s, 3H). ESIMS: m/z 459.0 [M+H]⁺.

### (v) N-(4-(4′-(Aminomethyl)-[1,1′-biphenyl]-3-yl)thiazol-2-yl)-4-(tert-butyl)benzamide (27)

tert-Butyl N-[[4-[3-[2-[(4-tert-butylbenzoyl)amino]thiazol-4-yl]phenyl]phenyl]methyl]carbamate (from step 2) (30 mg, 0.06 mmol) was dissolved in dry DCM and cooled to 0° C. Trifluoroacetic acid (0.085 mL, 1.11 mmol) was added drop wise to above solution and the reaction was stirred at rt for 1 hr. The reaction was diluted with aqueous NaHCO₃ (pH=8-9) and extracted with EtOAc. The organic layer was dried over anhydrous Na₂SO₄, filtered and concentrated under reduced pressure followed by purification on pre-packed Silica gel column on ISCO using 0-10% MeOH in CH₂Cl₂ (30 min) gave product. Yield: 13 mg (53%). ¹H NMR (CDCl₃) δ 7.99 (d, J=1.7 Hz, 1H), 7.90 (dd, J=8.7, 2.1 Hz, 2H), 7.71 (s, 1H), 7.58 (dd, J=8.3, 1.9 Hz, 2H), 7.52-7.46 (m, 3H), 7.44 (d, J=7.6 Hz, 1H), 7.34 (d, J=8.2 Hz, 2H), 7.26 (d, J=1.3 Hz, 1H), 7.18 (d, J=2.0 Hz, 1H), 3.85 (s, 2H), 1.29 (d, J=2.4 Hz, 9H). HR-ESIMS: m/z 442.1875 [M+H]⁺ calcd. for C₂₇H₂N₃OS, found 442.1952. HPLC purity: 99.5% (Retention Time=18.7 min).

### (vi) N-(4-(4′-Cyano-3′-hydroxy-[1,1′-biphenyl]-3-yl)thiazol-2-yl)-2-(methylthio)nicotinamide (22)

N-[4-[3-(4-cyano-3-methoxy-phenyl)phenyl]thiazol-2-yl]-2-methylsulfanyl-pyridine-3-carboxamide (70 mg, 0.15 mmol) was dissolved in anhydrous DCM (10 mL) and cooled to 0° C. Boron tribromide solution (0.76 mL, 0.76 mmol) was added dropwise to the cooled solution and after complete addition, ice bath was removed and the reaction mixture was stirred at rt for 36 hours. Reaction was quenched by adding MeOH (0.5 mL) and concentrated. Reaction mixture was suspended in deionized water (20 mL) and extracted with DCM (2×50 mL). The organic layer was separated, washed with aqueous NaCl, dried over anhydrous Na₂SO₄, and concentrated under reduced pressure followed by purification on pre-packed Silica gel column on ISCO using 0-10% MeOH in CH₂Cl₂ (30 min) gave product. Yield: 19 mg (27%). ¹H NMR (CDCl₃) δ 8.59 (dd, J=4.8, 1.8 Hz, 1H), 8.10 (dd, J=7.8, 1.8 Hz, 1H), 7.96 (s, 1H), 7.77 (d, J=7.1 Hz, 1H), 7.57 (d, J=8.1 Hz, 1H), 7.51-7.36 (m, 2H), 7.25 (d, J=1.5 Hz, 1H), 7.19 (dd, J=8.1, 1.6 Hz, 1H), 7.15 (d, J=1.4 Hz, 1H), 7.13 (dd, J=7.8, 4.8 Hz, 1H), 2.61 (s, 3H). HR-ESIMS: m/z 445.0715 [M+H]⁺ calcd. for C₂₃H₁₇N₄O₂S2, found 445.0792. HPLC purity: 98% (Retention Time=11.4 min).

### (vii) Methyl 4′-cyano-5-(2-(2-(methylthio)nicotinamido)thiazol-4-yl)-[1,1′-biphenyl]-3-carboxylate (20)

Purification on pre-packed Silica gel column on ISCO using 0-5% MeOH in CHCl₃ (30 min). Yield: 29 mg (77%). ¹H NMR (CDCl₃) δ 8.58 (dd, J=4.8, 1.8 Hz, 1H), 8.51 (t, J=1.6 Hz, 1H), 8.21 (dt, J=20.0, 1.7 Hz, 2H), 7.99 (dd, J=7.7, 1.7 Hz, 1H), 7.76 (d, J=1.0 Hz, 4H), 7.36 (s, 1H), 7.12 (dd, J=7.7, 4.8 Hz, 1H), 3.96 (s, 3H), 2.60 (s, 3H). HR-ESIMS: m/z 487.0820 [M+H]⁺ calcd. for C₂₅H₁₉N₄O₃S₂, found 487.0890. HPLC purity: 97% (Retention Time=18.6 min).

### f. Synthesis of 4′-cyano-5-(2-(2-(methylthio)nicotinamido)thiazol-4-yl)-[1,1′-biphenyl]-3-carboxylic Acid (21)

LiOH (4.43 mg, 0.18 mmol) was added to a Methyl 3-(4-cyanophenyl)-5-[2-[(2-methylsulfanylpyridine-3-carbonyl)amino]thiazol-4-yl]benzoate (20) (30 mg, 0.06 mmol) solution in THF-water (1:1) at 0° C. The reaction mixture was stirred at rt for 3 hrs and diluted with 1M HCl to form a precipitate. The precipitate was filtered off and washed several times with Hexanes-EtOAc (1:1) to a white solid. Yield: 14 mg (46%). ¹H NMR (CDCl₃) δ 13.26 (s, 1H), 12.92 (s, 1H), 8.70-8.58 (m, 2H), 8.51 (s, 1H), 8.22-8.10 (m, 2H), 8.08 (s, 1H), 8.04-7.91 (m, 4H), 7.26 (dd, J=7.8, 4.8 Hz, 1H), 2.47 (d, J=3.9 Hz, 3H). HR-ESIMS: m/z 473.0664 [M+H]⁺ calcd. for C₂₄H₁₇N₄O₃S₂, found 473.0725. HPLC purity: 95% (Retention Time=10.5 min).

### g. Synthesis of Compounds 37-46 and 48-52

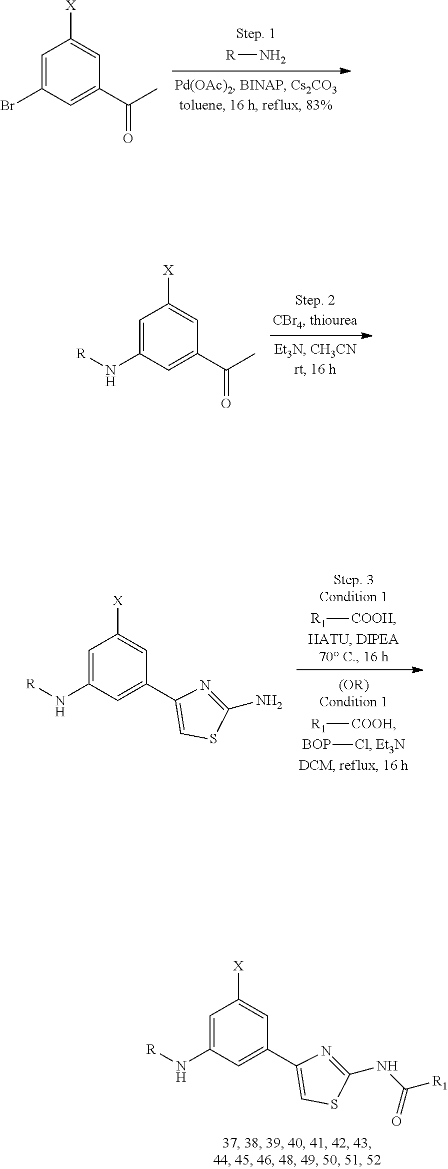

### i. Step 1: General Synthetic Procedure

Appropriate substituted aniline (1.2 mmol) was added to a anhydrous toluene (10 mL) solution of 3′-bromoacetophenone (1 mmol) followed by CsCO₃ (1.4 mmol), BINAP (0.08 mmol) and palladium(II) acetate (0.05 mmol) at room temperature under nitrogen. The reaction mixture was allowed to reflux overnight and checked for complete consumption of bromide by TLC analysis. The mixture was allowed to cool to room temperature, and filtered through a short pad of celite. The filtrate was diluted with water (50 mL) and extracted with EtOAc (2×100 mL). The combined organic layer was washed with brine, dried over anhydrous Na₂SO₄ and solvent was removed under reduced pressure followed by purification using pre-packed Silica gel column on ISCO to give product.

### (i) 4-((3-Acetylphenyl)amino)benzonitrile

Purification on pre-packed Silica gel column on ISCO using 0-10% MeOH in CH₂Cl₂ (30 min). Yield: 990 mg (83%). ¹H NMR (CDCl₃) δ 7.67-7.61 (m, 1H), 7.51-7.46 (m, 2H), 7.42-7.36 (m, 2H), 7.05-6.98 (m, 2H), 6.64 (d, J=8.8 Hz, 1H), 6.46 (s, 1H), 2.59 (s, 3H).

### (ii) 3-((3-Acetylphenyl)amino)benzonitrile

Purification on pre-packed Silica gel column on ISCO using 0-5% MeOH in CH₂Cl₂ (30 min). Yield: 410 mg (69%). ¹H NMR (CDCl₃) δ 7.68-7.62 (m, 1H), 7.61-7.55 (m, 1H), 7.41 (t, J=7.8 Hz, 1H), 7.35-7.28 (m, 2H), 7.26 (dd, J=2.2, 1.1 Hz, 2H), 7.19-7.15 (m, 1H), 7.02-6.97 (m, 1H), 2.59 (d, J=0.9 Hz, 3H). ESIMS: m/z 237.1 [M+H]⁺.

### (iii) 4-((3-Acetylphenyl)amino)-2-methoxybenzonitrile

Purification on pre-packed Silica gel column on ISCO using 0-5% MeOH in CH₂Cl₂ (30 min). Yield: 410 mg (61%). ¹H NMR (CDCl₃) δ 7.47 (dd, J=7.1, 1.4 Hz, 1H), 7.38-7.27 (m, 3H), 7.12 (s, 1H), 6.94 (d, J=8.7 Hz, 1H), 6.90-6.82 (m, 1H), 3.92 (s, 3H), 2.58 (d, J=9.3 Hz, 3H). ESIMS: m/z 267.1 [M+H]⁺.

### (iv) Tert-Butyl (4-((3-acetylphenyl)amino)benzyl)carbamate

Purification on pre-packed Silica gel column on ISCO using 0-50% EtOAc in Hexanes (30 min). Yield: 498 mg (58%). ¹H NMR (CDCl₃) δ 7.66-7.57 (m, 1H), 7.47 (dt, J=7.6, 1.3 Hz, 1H), 7.43-7.26 (m, 2H), 7.26-7.14 (m, 2H), 7.15-6.95 (m, 2H), 4.27 (d, J=5.6 Hz, 2H), 2.57 (s, 3H), 1.58-1.42 (m, 9H). ESIMS: m/z 285.1 [M-^{t}Bu]⁺.

### ii. Step 2: General Synthetic Procedure

To a solution of substituted acetophenone (1 mmol) in anhydrous CH₃CN was added carbon tetrabromide (1 mmol) followed by thiourea (1 mmol) and triethylamine (1 mmol). The reaction was allowed to stir at room temperature overnight. The completion of reaction was monitored by TLC and crude mass was diluted with water (50 mL) and extracted with EtOAc (2×100 mL). The combined organic layer was washed with brine, dried over anhydrous Na₂SO₄, and solvent was removed under reduced pressure followed by purification using pre-packed Silica gel column on ISCO gave product.

### (i) 4-((3-(2-Aminothiazol-4-yl)phenyl)amino)benzonitrile

Purification on pre-packed Silica gel column on ISCO using 0-60% EtOAc in Hexanes (30 min). Yield: 810 mg (55%). ¹H NMR (CDCl₃) δ 8.92 (s, 1H), 7.61-7.54 (m, 1H), 7.39-7.32 (m, 1H), 7.30 (t, J=7.8 Hz, 1H), 7.10-6.95 (m, 4H), 6.63-6.53 (m, 1H), 6.11 (s, 1H). ESIMS: m/z 293.1 [M+H]⁺.

### (ii) 2-((3-(2-Aminothiazol-4-yl)phenyl)amino)benzonitrile

Purification on pre-packed Silica gel column on ISCO using 0-10% MeOH in CH₂Cl₂ (30 min). Yield: 345 mg (47%). ¹H NMR (CDCl₃) δ 7.70 (dd, J=7.8, 1.6 Hz, 1H), 7.59-7.49 (m, 1H), 7.42 (t, J=7.8 Hz, 1H), 7.35 (dd, J=7.8, 1.6 Hz, 1H), 7.32-7.26 (m, 1H), 6.83-6.70 (m, 1H), 6.62-6.49 (m, 1H), 5.99 (s, 2H), 2.52 (d, J=16.2 Hz, 3H). ESIMS: m/z 293.1 [M+H]⁺.

### (iii) Tert-Butyl (4-((3-(2-aminothiazol-4-yl)phenyl)amino)benzyl)carbamate

Purification on pre-packed Silica gel column on ISCO using 0-50% EtOAc in Hexanes (30 min). Yield: 115 mg (49%). ¹H NMR (CDCl₃) δ 7.49-7.45 (m, 1H), 7.33-7.28 (m, 1H), 7.18 (d, J=8.2 Hz, 2H), 7.08-6.95 (m, 4H), 6.69 (s, 1H), 4.25 (d, J=5.7 Hz, 2H), 1.46 (s, 9H). ESIMS: m/z 397.1 [M+H]⁺.

### (iv) 5-((3-(2-Aminothiazol-4-yl)phenyl)amino)-2-methylbenzonitrile

Purification on pre-packed Silica gel column on ISCO using 0-5% MeOH in CH₂Cl₂ (30 min). Yield: 173 mg (28%). ¹H NMR (CDCl₃) δ 8.39 (s, 1H), 7.54-7.48 (m, 1H), 7.34-7.19 (m, 4H), 7.03-6.92 (m, 3H), 2.48-1.85 (m, 3H). ESIMS: m/z 307.0 [M+H]⁺.

### (v) 4-((3-(2-Aminothiazol-4-yl)-5-chlorophenyl)amino)benzonitrile

Purification on pre-packed Silica gel column on ISCO using 0-10% MeOH in CH₂Cl₂ (30 min). Yield: 110 mg (39%). ¹H NMR (CDCl₃) δ 7.54-7.41 (m, 2H), 7.41-7.35 (m, 1H), 7.35-7.28 (m, 1H), 7.12-7.02 (m, 2H), 6.70 (s, 1H). ESIMS: m/z 327.0 [M+H]⁺.

### (vi) 6-((3-(2-Aminothiazol-4-yl)phenyl)amino)nicotinonitrile

Purification on pre-packed Silica gel column on ISCO using 0-5% MeOH in CH₂Cl₂ (30 min). Yield: 255 mg (34%). ¹H NMR (CDCl₃) δ 8.50-8.40 (m, 1H), 7.75 (d, J=1.9 Hz, 1H), 7.65 (dd, J=9.1, 2.0 Hz, 1H), 7.54 (d, J=7.7 Hz, 1H), 7.38 (t, J=7.8 Hz, 1H), 7.35-7.27 (m, 1H), 6.82 (d, J=8.8 Hz, 1H), 6.75 (s, 1H). ESIMS: m/z 327.0 [M+H]⁺.

### iii. Step 3: General Synthetic Procedure

Condition 1. To a solution of carboxylic acid (1 mmol) in anhydrous DMF (5 mL) under nitrogen at room temperature, HATU (1.5 mmol) was added followed by the addition of DIPEA (3 mmol) and the reaction was stirred at same temperature for about 10 min. Appropriate substituted 2-aminothiazole was added to the reaction mixture and heated to 70° C. for 16 hrs. After completion of the reaction (by TLC analysis), the mixture was allowed to cool to rt. The reaction mixture was diluted with water (50 mL) and extracted with EtOAc (2×100 mL). The combined organic layer was washed with brine, dried over anhydrous Na₂SO₄ and solvent was removed under reduced pressure followed by purification using pre-packed Silica gel column on ISCO gave pure product.

Condition 2: To a solution of appropriate carboxylic acid (1 mmol) in anhydrous DCM (10 mL) under nitrogen at room temperature, BOP-Cl (1.5 mmol) was added followed by the addition of triethylamine (2.5 mmol) and the reaction was stirred at same temperature for about 10 min. Appropriate substituted 2-aminothiazole was added to the reaction and refluxed for 16 hrs. After completion of the reaction (TLC analysis), the mixture was cooled to room temperature, diluted with water (50 mL) and extracted with DCM (2×100 mL). The combined organic layer was washed with brine, dried over anhydrous Na₂SO₄, and solvent was removed under reduced pressure followed by purification using pre-packed Silica gel column on ISCO yielded pure product.

### (i) N-(4-(3-((4-Cyanophenyl)amino)phenyl)thiazol-2-yl)-2-(methylthio)nicotinamide (37)

This compound was synthesized by using condition 1. Purification on pre-packed Silica gel column on ISCO using 0-10% MeOH in CH₂Cl₂ (30 min). Yield: 69 mg (41%). ¹H NMR (CDCl₃) δ 8.48 (s, 1H), 8.41 (dd, J=4.8, 1.7 Hz, 1H), 7.69 (dd, J=7.7, 1.8 Hz, 1H), 7.57-7.38 (m, 3H), 7.35 (dt, J=7.7, 1.2 Hz, 1H), 7.33-7.24 (m, 1H), 7.06 (ddd, J=7.9, 2.2, 1.0 Hz, 1H), 7.04-6.90 (m, 2H), 6.85 (dd, J=7.7, 4.8 Hz, 1H), 6.25 (s, 1H), 2.51 (s, 3H). HR-ESIMS: m/z 444.0875 [M+H]⁺ calcd. for C₂₃H₁₈N₅OS₂, found 444.0941. HPLC purity: 95% (Retention Time=12.4 min).

### (ii) N-(4-(3-((3-Cyanophenyl)amino)phenyl)thiazol-2-yl)-2-(methylthio)nicotinamide (38)

This compound was synthesized by using condition 1. Purification on pre-packed Silica gel column on ISCO using 0-50% EtOAc in Hexanes (30 min). Yield: 51 mg (62%). ¹H NMR (CDCl₃) δ 8.46 (dd, J=4.8, 1.8 Hz, 1H), 7.77 (dd, J=7.7, 1.8 Hz, 1H), 7.45-7.42 (m, 1H), 7.37-7.27 (m, 3H), 7.27-7.26 (m, 1H), 7.20 (ddd, J=8.3, 2.4, 1.1 Hz, 1H), 7.13 (dt, J=7.5, 1.3 Hz, 1H), 7.03 (ddd, J=7.7, 2.2, 1.1 Hz, 1H), 6.92 (dd, J=7.7, 4.8 Hz, 1H), 5.98 (s, 1H), 2.55 (s, 3H). HR-ESIMS: m/z 444.0875 [M+H]⁺ calcd. for C₂₃H₁₅N₅OS₂, found 444.0941. HPLC purity: 96% (Retention Time=15.7 min).

### (iii) N-(4-(3-((2-Cyanophenyl)amino)phenyl)thiazol-2-yl)-2-(methylthio)nicotinamide (39)

This compound was synthesized by using condition 1. Purification on pre-packed Silica gel column on ISCO using 0-5% MeOH in CH₂Cl₂ (30 min). Yield: 14 mg (51%). ¹H NMR (CDCl₃) δ 10.67 (s, 1H), 8.53 (dd, J=4.8, 1.8 Hz, 1H), 7.91 (dd, J=7.7, 1.8 Hz, 1H), 7.61 (t, J=1.8 Hz, 1H), 7.56-7.45 (m, 2H), 7.37 (q, J=8.2, 7.8 Hz, 2H), 7.13 (dd, J=8.0, 1.3 Hz, 1H), 7.04 (dd, J=7.7, 4.8 Hz, 1H), 6.91-6.81 (m, 1H), 6.38 (s, 1H), 2.61 (s, 3H). HR-ESIMS: m/z 444.0875 [M+H]⁺ calcd. for C₂₃HisN₅OS₂, found 444.0942. HPLC purity: 96% (Retention Time=15.0 min).

### (iv) 2-(Methylthio)-N-(4-(3-((3-(trifluoromethyl)phenyl)amino)phenyl)thiazol-2-yl)nicotinamide (40)

This compound was synthesized by using condition 1. Purification on pre-packed Silica gel column on ISCO using 0-5% MeOH in CH₂Cl₂ (30 min). Yield: 55 mg (37%). ¹H NMR (CDCl₃) δ 10.64 (s, 1H), 8.53 (dd, J=4.8, 1.8 Hz, 1H), 7.91 (dd, J=7.7, 1.8 Hz, 1H), 7.63-7.53 (m, 1H), 7.52-7.37 (m, 3H), 7.33 (t, J=7.8 Hz, 1H), 7.20 (s, 1H), 7.12-6.99 (m, 4H), 2.61 (s, 3H). HR-ESIMS: m/z 487.0796 [M+H]⁺ calcd. for C₂₃H₁₅F₃N₄OS₂, found 487.0872. HPLC purity: 97% (Retention Time=15.3 min).

### (v) N-(4-(3-((4-yano-3-methoxyphenyl)amino)phenyl)thiazol-2-yl)-2-(methylthio)nicotinamide (46)

This compound was synthesized by using condition 2. Purification on pre-packed Silica gel column on ISCO using 0-5% MeOH in CH₂Cl₂ (30 min). Yield: 68 mg (93%). ¹H NMR (CDCl₃) 8.49-8.41 (m, 1H), 7.83-7.72 (m, 1H), 7.32-7.21 (m, 3H), 7.22-7.13 (m, 2H), 7.10 (s, 1H), 6.97 (dd, J=7.7, 4.8 Hz, 1H), 6.89-6.78 (m, 2H), 3.83 (s, 3H), 2.50 (s, 3H). HR-ESIMS: m/z 474.0980 [M+H]⁺ calcd. for C₂₄H₂₀N₅O₂S₂, found 474.1056. HPLC purity: 100% (Retention Time=15.7 min).

### (vi) N-(4-(3-((4-(aminomethyl)phenyl)amino)phenyl)thiazol-2-yl)-2-(methylthio)nicotinamide (45)

This compound was synthesized by using condition 1 followed by Boc group deprotection. Purification on pre-packed Silica gel column on ISCO using 0-40% EtOAc in Hexanes (30 min). Yield: 41 mg (57%). ¹H NMR (CDCl₃) δ 8.55 (dd, J=4.8, 1.8 Hz, 1H), 7.92 (s, 1H), 7.55 (s, 1H), 7.35-7.27 (m, 3H), 7.20 (d, J=9.1 Hz, 2H), 7.15 (s, 1H), 7.11-7.01 (m, 3H), 7.02-6.96 (m, 1H), 3.88 (s, 2H), 2.57 (s, 3H). HR-ESIMS: m/z 431.0995 [M-16]⁺ calcd. for C₂₃H₁₉N₄OS₂⁺, found 431.0000 [M-16]⁺. HPLC purity: 100% (Retention Time=11.3 min).

### (vii) 5-(Tert-Butyl)-N-(4-(3-((4-cyanophenyl)amino)phenyl)thiazol-2-yl)picolinamide (41)

This compound was synthesized by using condition 1. Purification on pre-packed Silica gel column on ISCO using 0-5% MeOH in CH₂Cl₂ (30 min). Yield: 59 mg (72%). ¹H NMR (CDCl₃) δ 8.70 (dd, J=2.3, 0.8 Hz, 1H), 8.19 (dd, J=8.2, 0.8 Hz, 1H), 7.90 (dd, J=8.2, 2.4 Hz, 1H), 7.71 (s, 1H), 7.58-7.52 (m, 1H), 7.53-7.29 (m, 4H), 7.20 (s, 1H), 7.13 (ddd, J=7.9, 2.3, 1.0 Hz, 1H), 7.03 (d, J=8.9 Hz, 2H), 1.39 (s, 9H). HR-ESIMS: m/z 454.1623 [M+H]⁺ calcd. for C₂₆H₂₄F₃N₅OS, found 454.1687. HPLC purity: 94% (Retention Time=18.7 min).

### (viii) N-(4-(3-((4-cyanophenyl)amino)phenyl)thiazol-2-yl)-4-(2-cyanopropan-2-yl)benzamide (44)

This compound was synthesized by using condition 1. Purification on pre-packed Silica gel column on ISCO using 0-5% MeOH in CH₂Cl₂ (30 min). Yield: 67 mg (85%). H NMR (CDCl₃) δ 7.96 (d, J=8.6 Hz, 2H), 7.69-7.55 (m, 3H), 7.55-7.43 (m, 3H), 7.37 (t, J=7.8 Hz, 2H), 7.11 (dd, J=7.6, 1.8 Hz, 1H), 7.06-6.92 (m, 2H), 1.77 (d, J=9.5 Hz, 6H). HR-ESIMS: m/z 464.1467 [M+H]⁺ calcd. for C₂₇H₂₂N₅OS, found 464.1548. HPLC purity: 100% (Retention Time=13.6 min).

### (ix) Methyl 3-((4-(3-((4-cyanophenyl)amino)phenyl)thiazol-2-yl)carbamoyl)bicyclo[1.1.1]pentane-1-carboxylate (43)

This compound was synthesized by using condition 1. Purification on pre-packed Silica gel column on ISCO using 0-5% MeOH in CH₂Cl₂ (30 min). Yield: 71 mg, (93%): ¹H NMR (400 MHz, CDCl₃) δ 8.80 (s, 1H), 7.67-7.63 (m, 1H), 7.55-7.48 (m, 3H), 7.40 (t, J=7.8 Hz, 1H), 7.17 (s, 1H), 7.13 (dd, J=7.9, 1.4 Hz, 1H), 7.04-6.98 (m, 2H), 3.72 (s, 3H), 2.43 (s, 6H). HR-ESIMS: m/z 445.1256[M+H]⁺ calcd for C₂₄H₂F₃N₄O₃S, found 445.3210. HPLC purity: 100% (Retention Time=11.2 min).

### (x) N-(4-(3-((3-cyano-4-methylphenyl)amino)phenyl)thiazol-2-yl)-2-(methylthio)nicotinamide (48)

This compound was synthesized by using condition 2. Purification on pre-packed Silica gel column on ISCO using 0-5% MeOH in CH₂Cl₂ (30 min). Yield: 33 mg, (38%): ¹H NMR (400 MHz, CDCl₃) δ 10.67 (s, 1H), 8.52 (dd, J=4.8, 1.8 Hz, 1H), 7.88 (dd, J=7.7, 1.8 Hz, 1H), 7.50-7.41 (m, 1H), 7.36 (dt, J=7.7, 1.3 Hz, 1H), 7.33-7.27 (m, 2H), 7.22-7.11 (m, 3H), 7.06-6.93 (m, 2H), 5.78 (s, 1H), 2.60 (s, 3H), 2.46 (s, 3H). HR-ESIMS: m/z 458.1031[M+H]⁺ calcd for C₂₄H₂₀N₅OS₂, found 458.11058. HPLC purity: 94.3% (Retention Time=16.5 min).

### (xi) N-(4-(3-chloro-5-((4-cyanophenyl)amino)phenyl)thiazol-2-yl)-2-(methylthio)nicotinamide (49)

This compound was synthesized by using condition 2. Purification on pre-packed Silica gel column on ISCO using 0-10% MeOH in CH₂Cl₂ (30 min). Yield: 9 mg, (28%): ¹H NMR (400 MHz, CDCl₃) δ 8.61 (dd, J=4.8, 1.7 Hz, 1H), 7.61-7.43 (m, 3H), 7.24 (d, J=15.8 Hz, 4H), 7.20-7.08 (m, 2H), 7.05 (d, J=8.7 Hz, 2H), 6.13 (s, 1H), 2.60 (d, J=35.6 Hz, 3H). HR-ESIMS: m/z 478.0485[M+H]⁺ calcd for C₂₃H₁₇ClN₅OS₂, found 478.05533. HPLC purity: 98% (Retention Time=16.9 min).

### (xii) N-(4-(3-((4-cyanophenyl)amino)phenyl)thiazol-2-yl)-4-methoxycyclohexane-1-carboxamide (50)

This compound was synthesized by using condition 2. Purification on pre-packed Silica gel column on ISCO using 0-5% MeOH in CH₂Cl₂ (30 min). Yield: 20 mg, (34%): ¹H NMR (400 MHz, CDCl₃) δ 9.52 (s, 1H), 7.66 (dt, J=6.9, 2.0 Hz, 2H), 7.60-7.57 (m, 1H), 7.52 (dt, J=7.8, 1.1 Hz, 1H), 7.50-7.44 (m, 2H), 7.38 (t, J=7.8 Hz, 1H), 7.11 (dd, J=7.9, 1.4 Hz, 1H), 7.03-6.92 (m, 2H), 3.30 (d, J=13.2 Hz, 6H), 2.54-1.03 (m, 20H). HR-ESIMS: m/z 433.1620[M+H]⁺ calcd for C₂₄H₂₅N₄O₂S, found 433.16956. HPLC purity: 94% (Retention Time=8.29, 12.14 min).

### (xiii) N-(4-(3-((5-cyanopyridin-2-yl)amino)phenyl)thiazol-2-yl)-2-(methylthio)nicotinamide (51)

This compound was synthesized by using condition 1. Purification on pre-packed Silica gel column on ISCO using 0-5% MeOH in CH₂Cl₂ (30 min). Yield: 20 mg, (34%): ¹H NMR (400 MHz, CDCl₃) δ 8.55 (dd, J=4.8, 1.7 Hz, 1H), 8.40 (d, J=1.6 Hz, 1H), 7.93 (dd, J=7.7, 1.7 Hz, 1H), 7.84 (t, J=1.8 Hz, 1H), 7.61 (dd, J=8.8, 2.3 Hz, 1H), 7.56-7.47 (m, 1H), 7.45-7.30 (m, 2H), 7.19 (s, 1H), 7.10 (dd, J=7.7, 4.8 Hz, 1H), 6.80 (d, J=8.9 Hz, 1H), 2.55 (s, 3H). HR-ESIMS: m/z 445.0827[M+H]⁺ calcd for C₂₂H₁₇N₆OS₂, found 445.08939. HPLC purity: 93.4% (Retention Time=14.6 min).

### (xiv) 3-cyano-N-(4-(3-((4-cyanophenyl)amino)phenyl)thiazol-2-yl)bicyclo[1.1.1]pentane-1-carboxamide (52)

This compound was synthesized by using condition 1. Purification on pre-packed Silica gel column on ISCO using 0-5% MeOH in CH₂Cl₂ (30 min). Yield: 21 mg, (34%): ¹H NMR (400 MHz, CDCl₃) δ 7.74-7.65 (m, 2H), 7.60-7.55 (m, 3H), 7.47 (d, J=8.9 Hz, 1H), 7.37 (s, 1H), 7.14 (s, 1H), 7.01 (d, J=8.9 Hz, 1H), 2.57 (s, 6H). HR-ESIMS: m/z 412.1154[M+H]⁺ calcd for C₂₃H₁₅N₅OS, found 412.12241. HPLC purity: 97% (Retention Time exo, endo mix=7.8, 14.6 min).

### (xv) 3-((4-(3-((4-Cyanophenyl)amino)phenyl)thiazol-2-yl)carbamoyl)bicyclo[1.1.1]pentane-1-carboxylic Acid (42)

Methyl 3-[[4-[3-(4-cyanoanilino)phenyl]thiazol-2-yl]carbamoyl]bicyclo[1.1.1]pentane-1-carboxylate (43) (35 mg, 0.08 mmol) was dissolved in THF-H₂O (1:1) and cooled to 0° C. To this solution was added powdered KOH (17.67 mg, 0.31 mmol) and the reaction was stirred at 0° C. for 10 mins then at rt for 30 mins. The reaction was quenched with 1M HCl to pH=7 and extracted with EtOAc (50 mL). The organic layer was washed with saturated aq. NaHCO₃ solution, dried over anhydrous Na₂SO₄, and concentrated under vacuum. The crude mass was diluted with DCM-MeOH (1:9) to obtain a clear solution which was titrated with hexanes to form a precipitate. Solid was filtered off and washed with DCM (3×5 mL) and dried under reduced pressure. Further purification on pre-packed Silica gel column on ISCO using 0-20% MeOH in CH₂Cl₂ (30 min) gave the pure product. Yield: 25 mg (74%). ¹H NMR (CD₃COOD) δ 7.59 (s, 1H), 7.55 (d, J=8.9 Hz, 2H), 7.41 (d, J=4.9 Hz, 2H), 7.29 (s, 1H), 7.22 (s, 1H), 7.17 (d, J=8.9 Hz, 2H), 2.52 (s, 6H). HR-ESIMS: m/z 431.1110 [M+H]⁺ calcd. for C₂₃H₁₉F₃N₄O₃S, found 431.11750. HPLC purity: 100% (Retention Time=12.19 min).

### h. Synthesis of Compounds 29-31, 34 and 35

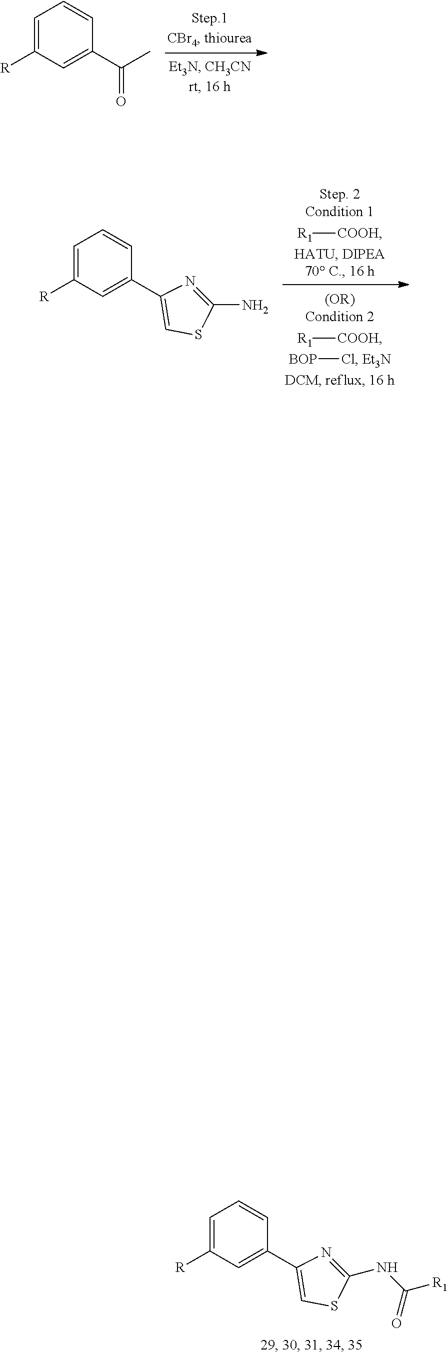

### i. Step 1: General Synthetic Procedure

To a solution of appropriate substituted acetophenone (1 mmol) in anhydrous acetonitrile (15 mL), carbon tetrabromide (1 mmol) was added followed by the addition of thiourea (1 mmol) and triethylamine (1 mmol). The reaction mixture was allowed to stir at room temperature overnight, after completion of the reaction (TLC analysis) it was diluted with water (50 mL) and extracted with EtOAc (2×100 mL). The combined organic layer was washed with brine, dried over anhydrous Na₂SO₄, and solvent was removed under reduced pressure followed by purification using pre-packed Silica gel column on ISCO yielded pure product.

### (i) 4-(3-(1H-Imidazol-1-yl)phenyl)thiazol-2-amine

Purification on pre-packed Silica gel column on ISCO using 0-10% MeOH in CH₂Cl₂ (30 min). Yield: 55 mg (21%). ¹H NMR (CDCl₃) δ 7.94-7.80 (m, 1H), 7.82-7.71 (m, 1H), 7.71-7.60 (m, 1H), 7.49-7.36 (m, 1H), 7.31 (d, J=1.3 Hz, 1H), 7.26 (d, J=1.6 Hz, 1H), 7.17-7.04 (m, 1H), 6.73 (d, J=1.1 Hz, 1H). ESIMS: m/z 243.1 [M+H]⁺.

### (ii) Tert-Butyl 4-(3-(2-aminothiazol-4-yl)phenyl)piperazine-1-carboxylate

Purification on pre-packed Silica gel column on ISCO using 0-5% MeOH in CH₂Cl₂ (30 min). Yield: 55 mg (21%). ¹H NMR (CDCl₃) δ 7.40 (d, J=1.0 Hz, 1H), 7.33-7.14 (m, 2H), 6.86 (d, J=2.9 Hz, 1H), 6.71 (d, J=1.3 Hz, 1H), 3.70-3.38 (m, 4H), 3.35-2.98 (m, 4H), 1.49 (s, 9H). ESIMS: m/z 305.1 [M-^{t}Bu]⁺.

### (iii) 4-(3-Morpholinophenyl)thiazol-2-amine

Purification on pre-packed Silica gel column on ISCO using 0-70% EtOAc in Hexanes (30 min). Yield: 95 mg. (37%). ¹H NMR (CDCl₃) δ 7.45-7.34 (m, 1H), 7.35-7.24 (m, 2H), 6.86 (d, J=6.6 Hz, 1H), 6.71 (s, 1H), 4.98 (s, 2H), 4.05-3.64 (m, 4H), 3.40-2.96 (m, 4H). ESIMS: m/z 262.1 [M+H]⁺.

### (iv) 1-(3-(2-Aminothiazol-4-yl)phenyl)pyrrolidin-2-one

Purification on pre-packed Silica gel column on ISCO using 0-10% MeOH in CH₂Cl₂ (30 min) (9:1). Yield: 60 mg (40%). ¹H NMR (CDCl₃) δ 7.86-7.72 (m, 1H), 7.53-7.38 (m, 2H), 7.30 (d, J=7.9 Hz, 1H), 6.63 (s, 1H), 3.96-3.77 (m, 2H), 2.63-2.42 (m, 2H), 2.22-2.01 (m, 2H). ESIMS: m/z 260.1 [M+H]⁺.

### ii. Step 2: General Synthetic Procedure

To a solution of carboxylic acid (1 mmol) in anhydrous DMF (10 mL) under nitrogen atmosphere and at room temperature, HATU (1.5 mmol) was added followed by the addition of DIPEA (3 mmol) and the reaction mass stirred at same temperature for about 10 min. Appropriate substituted 2-aminothiazoles was added and the reaction was heated to 70° C. for 16 hrs. After completion of reaction (TLC analysis), the mixture was allowed to cool to rt, diluted with water (50 mL) and extracted with EtOAc (2×100 mL). The combined organic layer was washed with brine, dried over anhydrous Na₂SO₄, and solvent was removed under reduced pressure followed by purification on pre-packed Silica gel column on ISCO gave the pure product.

### (i) N-(4-(3-(1H-imidazol-1-yl)phenyl)thiazol-2-yl)-2-(methylthio)nicotinamide (29)

Purification on pre-packed Silica gel column on ISCO using 0-40% EtOAc in Hexanes (35 min). Yield: 23 mg (39%). ¹H NMR (CDCl₃) δ 10.61 (s, 1H), 8.59 (dd, J=4.8, 1.8 Hz, 1H), 8.06 (dd, J=7.7, 1.8 Hz, 1H), 7.97-7.86 (m, 2H), 7.84-7.74 (m, 1H), 7.50 (t, J=7.9 Hz, 1H), 7.38-7.27 (m, 3H), 7.13 (dd, J=7.7, 4.8 Hz, 1H), 2.66 (s, 3H). HR-ESIMS: m/z 394.0718 [M+H]⁺ cald. for C₁₉H₁₆N₅O₂S₂, found 394.0793. HPLC purity: 99% (Retention Time=13.4 min).

### (ii) 4-(Tert-Butyl)-N-(4-(3-(piperazin-1-yl)phenyl)thiazol-2-yl)benzamide (30)

Step-2 followed by Boc group deprotection. To a dry DCM (3 mL) solution of tert-Butyl 4-[3-[2-[(4-tert-butylbenzoyl)amino]thiazol-4-yl]phenyl]piperazine-1-carboxylate (50 mg, 0.10 mmol) was added trifluoroacetic acid (0.22 mL, 2.88 mmol) drop wise at 0° C. and the reaction was stirred at same temperature for 1 h. The reaction was diluted with aq. NaHCO₃ and extracted with DCM (2×50 mL). The combined organic layer was dried over anhydrous Na₂SO₄, concentrated under reduced pressure to a solid material which was washed with Hexanes-EtOAc (9:1) resulted in a pure product. Yield: 28 mg (69%). ¹H NMR (CDCl₃) δ 8.10-8.04 (m, 2H), 7.63 (s, 1H), 7.58-7.54 (m, 2H), 7.50 (s, 1H), 7.34 (d, J=7.7 Hz, 1H), 7.24 (t, J=7.9 Hz, 1H), 6.87 (dd, J=8.0, 1.9 Hz, 1H), 3.13-3.05 (m, 4H), 2.86 (d, J=4.7 Hz, 4H), 1.31 (s, 9H). HR-ESIMS: m/z 421.1984 [M+H]⁺ calcd. for C₂₄H₂₉N₄S, found 421.2057. HPLC purity: 99% (Retention Time=9.1 min).

### (iii) 2-(Methylthio)-N-(4-(3-(piperazin-1-yl)phenyl)thiazol-2-yl)nicotinamide (31)

Step-2 followed by Boc group deprotection. To a dry DCM (3 mL) solution of tert-Butyl 4-[3-[2-[(4-tert-butylbenzoyl)amino]thiazol-4-yl]phenyl]piperazine-1-carboxylate (50 mg, 0.10 mmol) was added trifluoroacetic acid (0.22 mL, 2.88 mmol) drop wise at 0° C. and reaction and mixture was stirred at the same temperature for 1 h. The reaction was diluted with saturated aqueous NaHCO₃ and extracted with DCM (2×50 mL). The combined organic layer was dried over anhydrous Na₂SO₄, concentrated under reduced pressure and crude material was purified using purification on pre-packed Silica gel column on ISCO using 0-30% MeOH in CHCl₃ (45 min) to gave product. Yield: 32 mg (76%). ¹H NMR (CDCl₃) δ 8.53 (dd, J=4.8, 1.8 Hz, 1H), 7.92 (dd, J=7.7, 1.8 Hz, 1H), 7.41-7.31 (m, 1H), 7.25 (d, J=5.4 Hz, 2H), 7.14 (s, 1H), 7.07 (dd, J=7.7, 4.8 Hz, 1H), 6.87 (d, J=6.8 Hz, 1H), 3.17 (dd, J=6.2, 3.9 Hz, 4H), 3.00 (dd, J=6.1, 3.9 Hz, 4H), 2.55 (s, 3H). HR-ESIMS: m/z 412.1188 [M+H]⁺ calcd. for C₂₀H₂₂N₅OS₂, found 412.1255 HPLC purity: 96% (Retention Time=4.9 min).

### (iv) 2-(Methylthio)-N-(4-(3-morpholinophenyl)thiazol-2-yl)nicotinamide (34)

Purification using Purification on pre-packed Silica gel column on ISCO using 0-5% MeOH in CH₂Cl₂ (18 min). Yield: 29 mg (79%). ¹H NMR (CDCl₃) δ 8.54 (dd, J=4.8, 1.8 Hz, 1H), 7.93 (dd, J=7.7, 1.7 Hz, 1H), 7.31 (d, J=25.5 Hz, 3H), 7.14 (s, 1H), 7.09 (dd, J=7.7, 4.8 Hz, 1H), 6.86 (dt, J=6.6, 2.5 Hz, 1H), 3.93-3.73 (m, 4H), 3.26-3.09 (m, 4H), 2.56 (s, 3H). HR-ESIMS: m/z 413.1028 [M+H]⁺ cald. for C₂H₂N₄O₂S₂, found 413.1094. HPLC purity: 100% (Retention Time=6.9 min).

### (v) 2-(Methylthio)-N-(4-(3-(2-oxopyrrolidin-1-yl)phenyl)thiazol-2-yl)nicotinamide (35)

Purification on pre-packed Silica gel column on ISCO using 0-50% EtOAc in Hexanes (35 min). Yield: 15 mg (30%). ¹H NMR (CDCl₃) δ 8.56 (dd, J=4.8, 1.8 Hz, 1H), 8.08 (t, J=1.8 Hz, 1H), 7.98 (dd, J=7.7, 1.8 Hz, 1H), 7.57 (dd, J=8.1, 1.4 Hz, 2H), 7.40 (d, J=8.1 Hz, 1H), 7.23 (s, 1H), 7.09 (dd, J=7.7, 4.8 Hz, 1H), 4.00-3.81 (m, 2H), 2.62 (d, J=4.3 Hz, 3H), 2.27-2.10 (m, 2H), 1.26 (t, J=7.1 Hz, 2H). HR-ESIMS: m/z 411.0871 [M+H]⁺ calcd. for C₂₀H₁₉N₄O₂S₂, found 411.0951. HPLC purity: 98% (Retention Time=13 min).

### i. Synthesis of Compounds 32 and 33

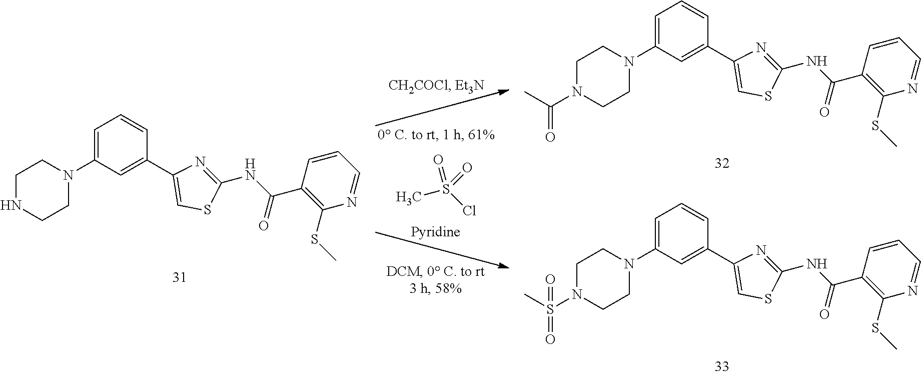

### i. N-(4-(3-(4-acetylpiperazin-1-yl)phenyl)thiazol-2-yl)-2-(methylthio)nicotinamide (32)

To a dry DCM (5 mL) solution of methylsulfanyl-N-[4-(3-piperazin-1-ylphenyl)thiazol-2-yl]pyridine-3-carboxamide (30 mg, 0.07 mmol) at 0° C. was added acetyl chloride (0.0062 mL, 0.09 mmol) followed by triethylamine hydrochloride (0.042 mL, 0.22 mmol) and the reaction mixture was stirred at room temperature for 1 hour. The reaction was diluted with deionized water (50 mL) and extracted with DCM (3×50 mL). The combined organic layer was dried over anhydrous Na₂SO₄, concentrated under reduced pressure followed by purification on pre-packed Silica gel column on ISCO using 0-10% MeOH in CH₂Cl₂ (30 min) gave the pure product. Yield: 21 mg (61%). ¹H NMR (CDCl₃) δ 8.53 (dd, J=4.8, 1.8 Hz, 1H), 7.93 (dd, J=7.7, 1.8 Hz, 1H), 7.43-7.31 (m, 1H), 7.32-7.22 (m, 3H), 7.03 (dd, J=7.7, 4.8 Hz, 1H), 6.86 (d, J=3.4 Hz, 1H), 3.87-3.70 (m, 2H), 3.72-3.53 (m, 2H), 3.21 (dt, J=10.6, 5.2 Hz, 4H), 2.61 (s, 3H). HR-ESIMS: m/z 454.1293 [M+H]⁺ calcd. for C₂₂H₂₄N₅O₂S₂, found 454.1360. HPLC purity: 96.6% (Retention Time=6.4 min).

### ii. N-(4-(3-(4-(methylsulfonyl)piperazin-1-yl)phenyl)thiazol-2-yl)-2-(methylthio)nicotinamide (33)

To a dry DCM (10 mL) solution of 2-methylsulfanyl-N-[4-(3-piperazin-1-ylphenyl)thiazol-2-yl]pyridine-3-carboxamide (25 mg, 0.06 mmol) at 0° C. was added methanesulfonyl chloride (8.35 mg, 0.07 mmol) followed by pyridine (9.61 mg, 0.12 mmol) and the reaction mixture was stirred at room temperature for 3 h. The reaction was diluted with water and extracted with DCM (2×50 mL). The combined organic layer was washed with brine, dried over anhydrous Na₂SO₄, concentrated under reduced pressure followed by purification on pre-packed Silica gel column on ISCO using 0-100% EtOAc in Hexanes (40 min) gave pure product. Yield: 18 mg (58%). ¹H NMR (CDCl₃) δ 10.59 (s, 1H), 8.54 (dd, J=4.8, 1.7 Hz, 1H), 7.93 (dd, J=7.7, 1.8 Hz, 1H), 7.43-7.35 (m, 1H), 7.35-7.26 (m, 2H), 7.19 (s, 1H), 7.04 (dd, J=7.7, 4.8 Hz, 1H), 6.88 (d, J=6.3 Hz, 1H), 3.40 (dd, J=6.4, 2.9 Hz, 4H), 3.36-3.25 (m, 4H), 2.84 (s, 3H), 2.62 (s, 3H). HR-ESIMS: m/z 490.0963 [M+H]⁺ calcd. for C₂₁H₂₄N₅O₃S₃, found 490.1035. HPLC purity: 95% (Retention Time=15.5 min).

### j. Synthesis of N-(4-(3-((4-cyanophenyl)amino)-5-morpholinophenyl)thiazol-2-yl)-2-(methylthio)nicotinamide (47)

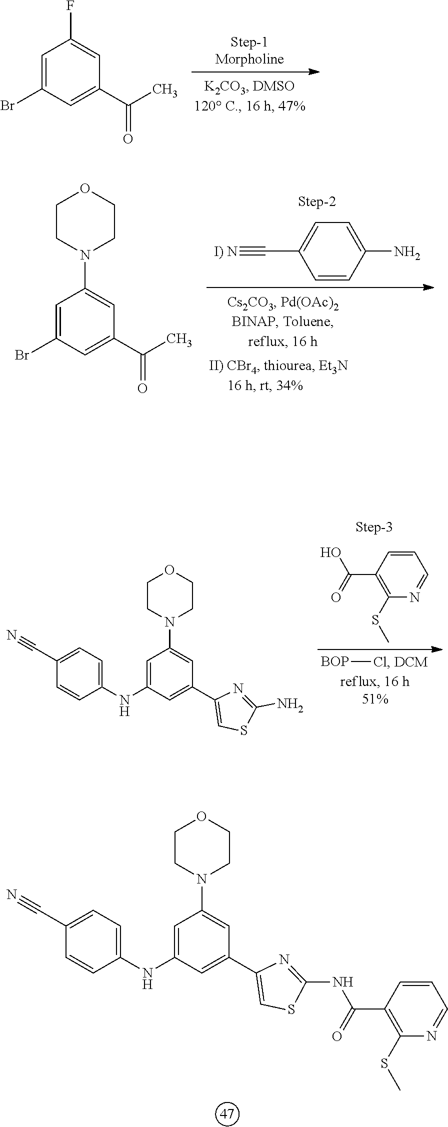

### i. Step 1: 1-(3-Bromo-5-morpholinophenyl)ethan-1-one

To an anhydrous DMSO (15 mL) solution of 1-(3-bromo-5-fluoro-phenyl)ethanone (0.5 g, 2.3 mmol) under nitrogen was added morphiline (0.3 mL, 3.46 mmol) followed by anhydrous potassium carbonate (0.49 g, 3.46 mmol) and the reaction was heated at 130° C. for 16 hrs. The reaction was diluted with deionized water and extracted with EtOAc (2×100 mL). The combined organic layer was dried over anhydrous Na₂SO₄, concentrated under reduced pressure followed by purification on ISCO system gave the pure product. Purification on pre-packed Silica gel column on ISCO using 0-50% EtOAc in Hexanes (30 min). Yield: 310 mg (47%). ¹H NMR (400 MHz, CDCl₃) δ 7.51 (t, J=1.5 Hz, 1H), 7.42-7.37 (m, 1H), 7.21-7.17 (m, 1H), 3.86 (dd, J=5.8, 4.0 Hz, 4H), 3.21 (dd, J=5.7, 4.1 Hz, 4H), 2.57 (d, J=0.4 Hz, 3H). ESIMS: m/z 286.1 [M+2].⁺

### ii. Step 2: 4-((3-(2-aminothiazol-4-yl)-5-morpholinophenyl)amino)benzonitrile

To an anydrous toluene (10 mL) solution of 1-(3-bromo-5-morpholino-phenyl)ethanone (144 mg, 0.51 mmol) was added 4-aminobenzonitrile (72 mg, 0.61 mmol), cesium carbonate (234 mg, 0.71 mmol), BINAP (25 mg, 0.04 mmol) and palladium(II) acetate (5.8 mg, 0.03 mmol) at room temperature under nitrogen. The reaction mixture was refluxed overnight and once the TLC analysis of reaction showed consumption of the bromide, it was cooled to room temperature. The reaction mixture was filtered through a short pad of celite. The filtrate was diluted with water and extracted with EtOAc (2×50 mL). The combined organic layer was washed with brine, dried over anhydrous Na₂SO₄ and solvent was removed under reduced pressure. This material was dissolved in anhydrous acetonitrile (10 mL) and to this solution, carbon tetrabromide (168 mg, 0.51 mmol) was added followed by thiourea (39 mg, 0.51 mmol), triethylamine (0.096 mL, 0.51 mmol). The reaction mixture was stirred at room temperature, diluted with water (50 mL) and extracted with EtOAc (2×100 mL). The organic layer was washed with brine, dried over anhydrous Na₂SO₄ and solvent was removed under reduced pressure. Purification of crude on pre-packed Silica gel column on ISCO using 0-30% EtOAc in Hexanes (30 min) gave the product. Yield: 66 mg (34%). ¹H NMR (CDCl₃) δ 7.56-7.42 (m, 2H), 7.17-7.05 (m, 2H), 6.98 (d, J=8.7 Hz, 2H), 6.71 (s, 1H), 6.63 (t, J=2.1 Hz, 1H), 6.01 (d, J=6.5 Hz, 1H), 4.92 (s, 2H), 3.91-3.76 (m, 4H), 3.27-3.09 (m, 4H). ESIMS: m/z 377.1 [M+H]⁺.

### iii. Step 3: n-(4-(3-((4-cyanophenyl)amino)-5-morpholinophenyl)thiazol-2-yl)-2-(methylthio)nicotinamide

To an anhydrous DCM (5 mL) solution of 2-(methylthio)nicotinic acid (15 mg, 0.09 mmol) under nitrogen at room temperature, BOP-Cl (34 mg, 0.13 mmol) was added followed by the addition of triethylamine (0.042 mL, 0.22 mmol) and the reaction mixture was stirred at same temperature for about 10 min. 4-[3-(2-Aminothiazol-4-yl)-5-morpholino-anilino]benzonitrile (33.46 mg, 0.09 mmol) from Step-2 was added to the reaction mixture and refluxed for 16 hrs. The reaction was allowed to cool to room temperature, diluted with water (50 mL) and extracted with DCM (2×100 mL). The organic layer was washed with brine, dried over anhydrous Na₂SO₄, and solvent was removed under reduced pressure. Purification of crude material on pre-packed Silica gel column on ISCO using 0-10% MeOH in CH₂Cl₂ (30 min) gave the product. Yield: 24 mg (51%). ¹H NMR (CDCl₃) δ 10.40 (s, 1H), 8.56 (dd, J=4.8, 1.8 Hz, 1H), 7.95 (dd, J=7.7, 1.8 Hz, 1H), 7.56-7.41 (m, 2H), 7.18 (s, 1H), 7.11 (d, J=2.0 Hz, 2H), 7.07 (dd, J=7.7, 4.8 Hz, 1H), 7.02-6.94 (m, 2H), 6.63 (t, J=2.1 Hz, 1H), 6.06 (s, 1H), 3.98-3.70 (m, 4H), 3.33-3.07 (m, 4H), 2.63 (s, 3H). HR-ESIMS: m/z 529.1402 [M+H]⁺ calcd. for C₂₇H₂₅N₆O₂S₂, found 529.1466. HPLC purity: 100% (Retention Time=10.2 min).

### k. Synthesis of N-(4-(4′-(aminomethyl)-[1,1′-biphenyl]-3-yl)thiazol-2-yl)-2-(methylthio)nicotinamide (26)

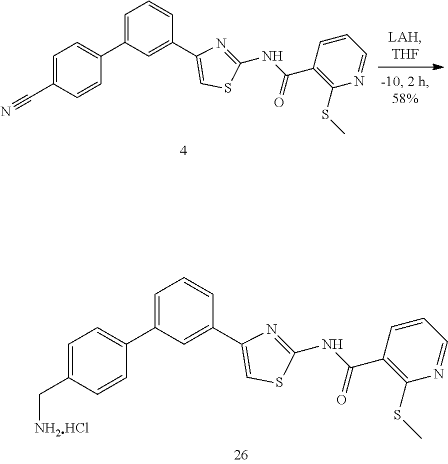

N-[4-[3-(4-cyanophenyl)phenyl]thiazol-2-yl]-2-methylsulfanyl-pyridine-3-carboxamide (4) (50 mg, 0.12 mmol) was dissolved in anhydrous THF (3 mL) and to this solution at −10° C., Lithium aluminum hydride (8.86 mg, 0.23 mmol) was added portionwise. The reaction mixture was stirred at the same temperature for about 2 hrs. The reaction was quenched with aqueous saturated NH₄Cl solution and extracted with EtOAc (2×100 mL). The combined organic layer was dried over anhydrous Na₂SO₄ and concentrated under reduced pressure. The crude amine was converted to HCl salt using 4M HCl in 1,4-dioxane. To a 1,4-dioxane (1 mL) solution of crude amine at 0° C. was added 4M HCl hydrochloric acid (1.0 mL) and the reaction mixture was stirred at same temperature for 30 min. The solid formed was collected by filtration, titurated with Hexane-EtOAc (1:1) and resulted in pure product which was dried under reduced pressure. Yield: 33 mg (58%). ¹H NMR (CDCl₃) δ 12.85 (s, 1H), 8.61 (dd, J=4.8, 1.7 Hz, 1H), 8.41 (s, 3H), 8.23 (t, J=1.6 Hz, 1H), 8.12 (dd, J=7.7, 1.7 Hz, 1H), 7.94 (d, J=7.9 Hz, 1H), 7.88 (s, 1H), 7.77 (d, J=8.3 Hz, 2H), 7.72-7.46 (m, 4H), 7.25 (dd, J=7.7, 4.8 Hz, 1H), 4.06 (q, J=5.9 Hz, 2H), 2.47 (d, J=1.8 Hz, 3H). HR-ESIMS: m/z 433.1079 [M+H]⁺ calcd. for C₂₃H₂₁N₄OS₂, found 433.1162. HPLC purity: 95% (Retention Time=14 min).

### 2. Characterization of Antiviral Agents

A list of compounds evaluated for antiviral activity against CHIKV is shown in Table 1 below.

TABLE 1

| | | | VTR @ | | |
|---|---|---|---|---|---|
| | | | 10 μM | EC₉₀ | CC₅₀ |
| No. | Structure | MW | (logs) | (μM) | (μM) |
| 1 | 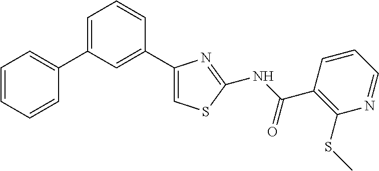 [SMILES: CSc1ncccc1C(=O)Nc1nc(-c2cccc(-c3ccccc3)c2)cs1] | 403.52 | 7.82 | 0.38 | >40 |
| 2 | 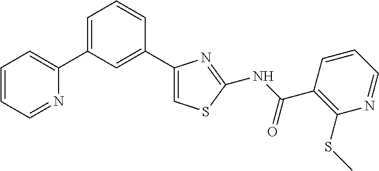 [SMILES: CSc1ncccc1C(=O)Nc1nc(-c2cccc(-c3ccccn3)c2)cs1] | 404.51 | 3.97 | 0.7 | >30 |
| 3 | 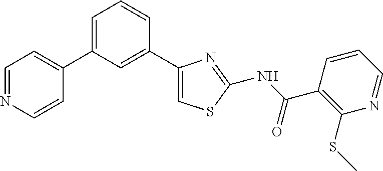 [SMILES: CSc1ncccc1C(=O)Nc1nc(-c2cccc(-c3ccncc3)c2)cs1] | 404.51 | <1 | ND | >40 |
| 4 | 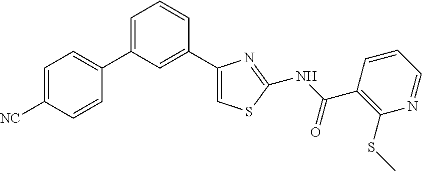 [SMILES: CSc1ncccc1C(=O)Nc1nc(-c2cccc(-c3ccc(C#N)cc3)c2)cs1] | 428.53 | 4.73 | 0.31 | >40 |
| 5 | 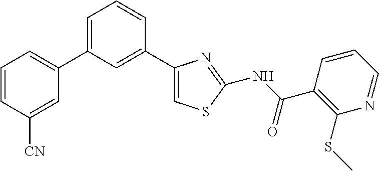 [SMILES: CSc1ncccc1C(=O)Nc1nc(-c2cccc(-c3cccc(C#N)c3)c2)cs1] | 428.53 | 5.25 | 0.098 | >40 |
| 6 | 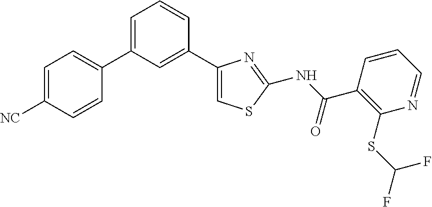 [SMILES: N#Cc1ccc(-c2cccc(-c3csc(NC(=O)c4cccnc4SC(F)F)n3)c2)cc1] | 464.51 | 7.6 | ND | >30 |
| 7 | 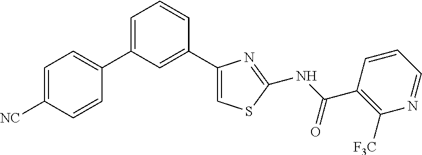 [SMILES: N#Cc1ccc(-c2cccc(-c3csc(NC(=O)c4cccnc4C(F)(F)F)n3)c2)cc1] | 450.44 | 5.09 | ND | 8.07 |
| 8 | 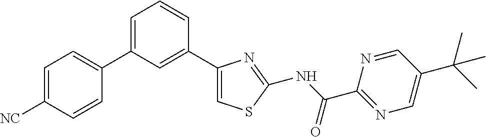 [SMILES: CC(C)(C)c1cnc(C(=O)Nc2nc(-c3cccc(-c4ccc(C#N)cc4)c3)cs2)nc1] | 439.53 | <1 | ND | >40 |
| 9 | 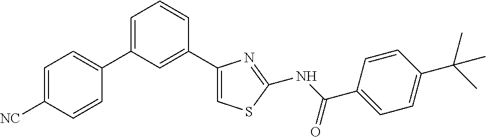 [SMILES: CC(C)(C)c1ccc(C(=O)Nc2nc(-c3cccc(-c4ccc(C#N)cc4)c3)cs2)cc1] | 437.56 | 4.68 | 0.28 | >40 |
| 10 | 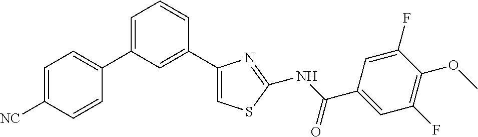 [SMILES: COc1c(F)cc(C(=O)Nc2nc(-c3cccc(-c4ccc(C#N)cc4)c3)cs2)cc1F] | 447.46 | 7.6 | 0.31 | >30 |
| 11 | 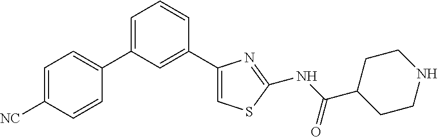 [SMILES: N#Cc1ccc(-c2cccc(-c3csc(NC(=O)C4CCNCC4)n3)c2)cc1] | 388.49 | 8.5 | ND | >30 |
| 12 | 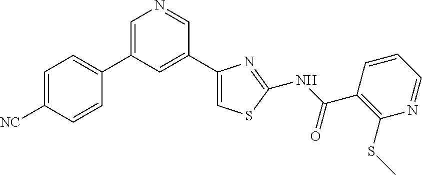 [SMILES: CSc1ncccc1C(=O)Nc1nc(-c2cncc(-c3ccc(C#N)cc3)c2)cs1] | 429.52 | <1 | ND | >40 |
| 13 | 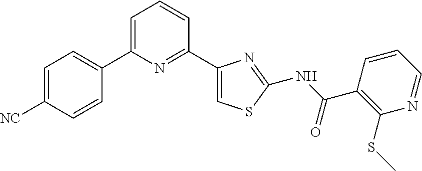 [SMILES: CSc1ncccc1C(=O)Nc1nc(-c2cccc(-c3ccc(C#N)cc3)n2)cs1] | 429.52 | 2.72 | ND | 16.34 |
| 14 | 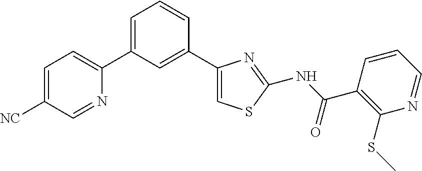 [SMILES: CSc1ncccc1C(=O)Nc1nc(-c2cccc(-c3ccc(C#N)cn3)c2)cs1] | 429.52 | 6 | 0.62 | >40 |
| 15 | 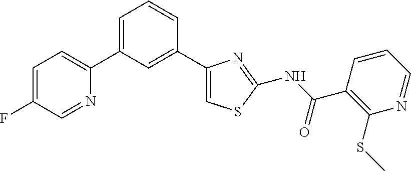 [SMILES: CSc1ncccc1C(=O)Nc1nc(-c2cccc(-c3ccc(F)cn3)c2)cs1] | 422.5 | 5.64 | 0.33 | >40 |
| 16 | 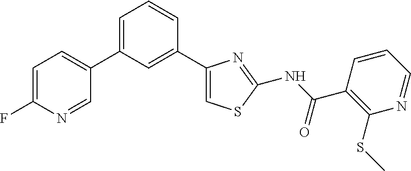 [SMILES: CSc1ncccc1C(=O)Nc1nc(-c2cccc(-c3ccc(F)nc3)c2)cs1] | 422.5 | 6.09 | 0.33 | >30 |
| 17 | 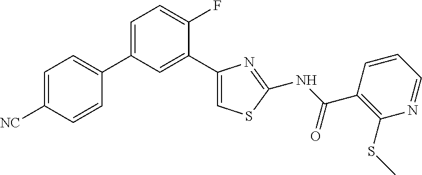 [SMILES: CSc1ncccc1C(=O)Nc1nc(-c2cc(-c3ccc(C#N)cc3)ccc2F)cs1] | 446.52 | 5.75 | 0.34 | 25 |
| 18 | 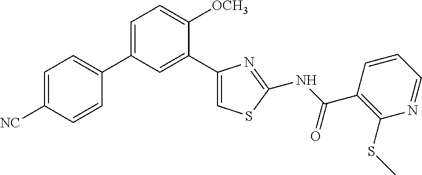 [SMILES: COc1ccc(-c2ccc(C#N)cc2)cc1-c1csc(NC(=O)c2cccnc2SC)n1] | 458.56 | 1.73 | ND | >40 |
| 19 | 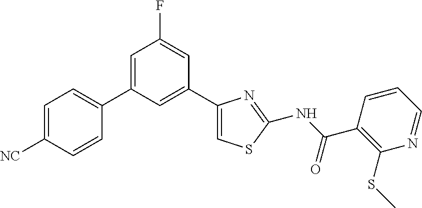 [SMILES: CSc1ncccc1C(=O)Nc1nc(-c2cc(F)cc(-c3ccc(C#N)cc3)c2)cs1] | 446.52 | 6.1 | 0.19 | >30 |
| 20 | 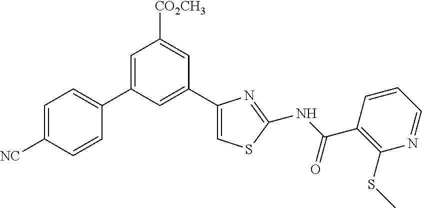 [SMILES: COC(=O)c1cc(-c2ccc(C#N)cc2)cc(-c2csc(NC(=O)c3cccnc3SC)n2)c1] | 486.57 | 3.23 | ND | 17.62 |
| 21 | 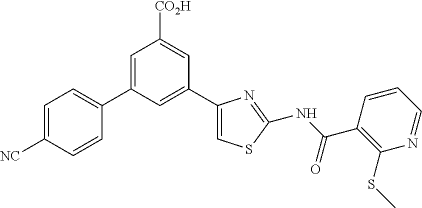 [SMILES: CSc1ncccc1C(=O)Nc1nc(-c2cc(C(=O)O)cc(-c3ccc(C#N)cc3)c2)cs1] | 472.54 | <1 | ND | >40 |
| 22 | 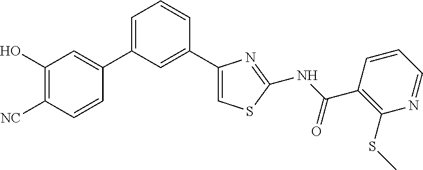 [SMILES: CSc1ncccc1C(=O)Nc1nc(-c2cccc(-c3ccc(C#N)c(O)c3)c2)cs1] | 444.53 | 8.5 | 1.6 | >30 |
| 23 | 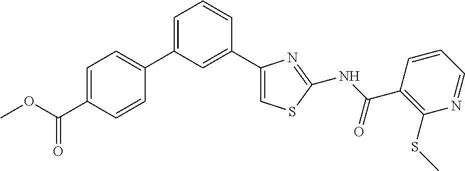 [SMILES: COC(=O)c1ccc(-c2cccc(-c3csc(NC(=O)c4cccnc4SC)n3)c2)cc1] | 461.56 | 8.69 | 0.822 | >40 |
| 24 | 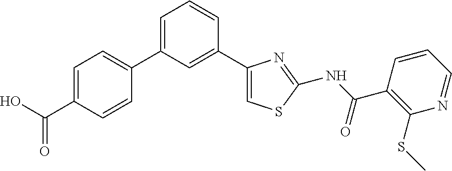 [SMILES: CSc1ncccc1C(=O)Nc1nc(-c2cccc(-c3ccc(C(=O)O)cc3)c2)cs1] | 447.53 | 1.49 | ND | >40 |
| 25 | 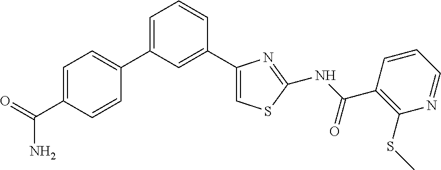 [SMILES: CSc1ncccc1C(=O)Nc1nc(-c2cccc(-c3ccc(C(N)=O)cc3)c2)cs1] | 446.54 | 2.43 | 0.21 | 17.9 |
| 26 | 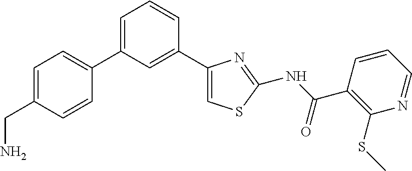 [SMILES: CSc1ncccc1C(=O)Nc1nc(-c2cccc(-c3ccc(CN)cc3)c2)cs1] | 432.10 | 8.75 | 0.75 | >40 |
| 27 | 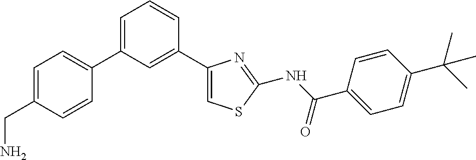 [SMILES: CC(C)(C)c1ccc(C(=O)Nc2nc(-c3cccc(-c4ccc(CN)cc4)c3)cs2)cc1] | 441.59 | 5.24 | 1.27 | 9.02 |
| 28 | 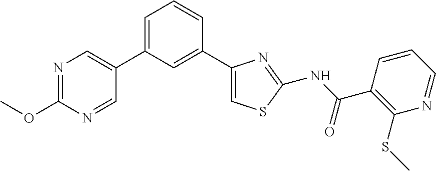 [SMILES: COc1ncc(-c2cccc(-c3csc(NC(=O)c4cccnc4SC)n3)c2)cn1] | 435.52 | 2.52 | ND | >40 |
| 29 | 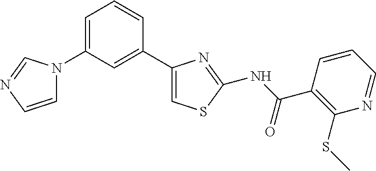 [SMILES: CSc1ncccc1C(=O)Nc1nc(-c2cccc(-n3ccnc3)c2)cs1] | 393.49 | 1.39 | 0.27 | >40 |
| 30 | 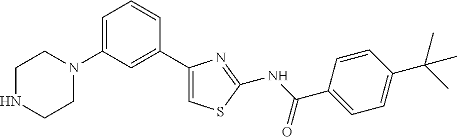 [SMILES: CC(C)(C)c1ccc(C(=O)Nc2nc(-c3cccc(N4CCNCC4)c3)cs2)cc1] | 420.57 | 5.57 | ND | 4.1 |
| 31 | 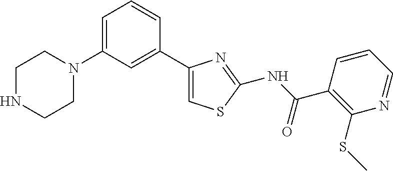 [SMILES: CSc1ncccc1C(=O)Nc1nc(-c2cccc(N3CCNCC3)c2)cs1] | 411.54 | 8.69 | 9.1 | >30 |
| 32 | 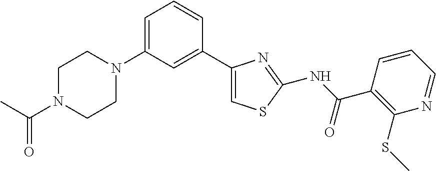 [SMILES: CSc1ncccc1C(=O)Nc1nc(-c2cccc(N3CCN(C(C)=O)CC3)c2)cs1] | 453.58 | <1 | ND | >40 |
| 33 | 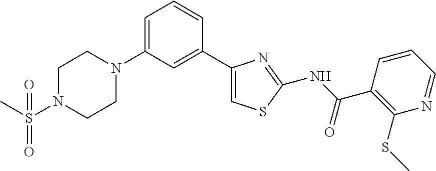 [SMILES: CSc1ncccc1C(=O)Nc1nc(-c2cccc(N3CCN(S(C)(=O)=O)CC3)c2)cs1] | 489.63 | 2.04 | ND | >40 |
| 34 | 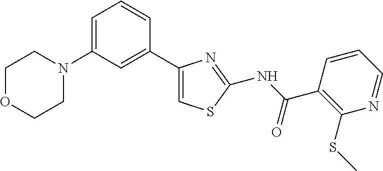 [SMILES: CSc1ncccc1C(=O)Nc1nc(-c2cccc(N3CCOCC3)c2)cs1] | 412.53 | 1.88 | ND | >40 |
| 35 | 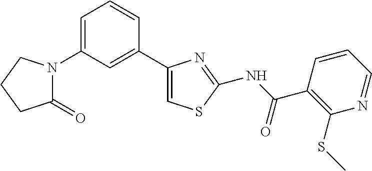 [SMILES: CSc1ncccc1C(=O)Nc1nc(-c2cccc(N3CCCC3=O)c2)cs1] | 410.51 | <1 | ND | 39.4 |
| 36 | 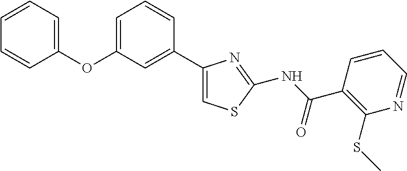 [SMILES: CSc1ncccc1C(=O)Nc1nc(-c2cccc(Oc3ccccc3)c2)cs1] | 419.52 | 4.87 | ND | >40 |
| 37 | 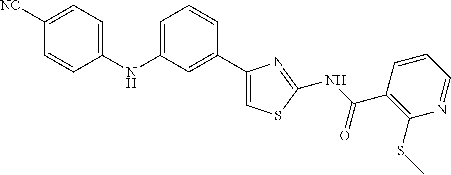 [SMILES: CSc1ncccc1C(=O)Nc1nc(-c2cccc(Nc3ccc(C#N)cc3)c2)cs1] | 443.54 | 8.68 | 0.45 | >40 |
| 38 | 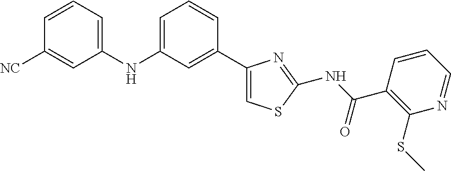 [SMILES: CSc1ncccc1C(=O)Nc1nc(-c2cccc(Nc3cccc(C#N)c3)c2)cs1] | 443.54 | 5.72 | 0.15 | >40 |
| 39 | 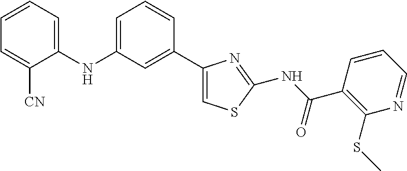 [SMILES: CSc1ncccc1C(=O)Nc1nc(-c2cccc(Nc3ccccc3C#N)c2)cs1] | 443.54 | 5.23 | 0.50 | >40 |
| 40 | 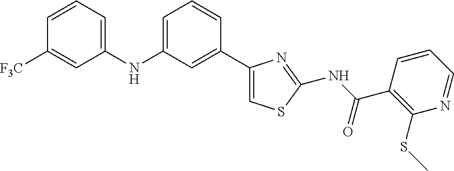 [SMILES: CSc1ncccc1C(=O)Nc1nc(-c2cccc(Nc3cccc(C(F)(F)F)c3)c2)cs1] | 486.53 | 5.36 | 3.1 | >40 |
| 41 | 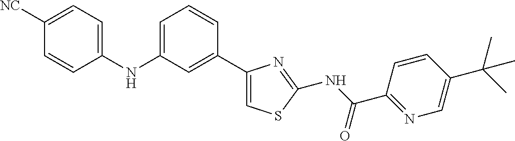 [SMILES: CC(C)(C)c1ccc(C(=O)Nc2nc(-c3cccc(Nc4ccc(C#N)cc4)c3)cs2)nc1] | 453.56 | 0.82 | >10 | >40 |
| 42 | 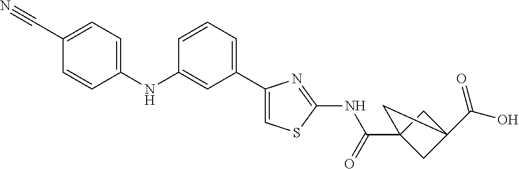 [SMILES: N#Cc1ccc(Nc2cccc(-c3csc(NC(=O)C45CC(C(=O)O)(C4)C5)n3)c2)cc1] | 430.11 | 0 | >10 | >40 |
| 43 | 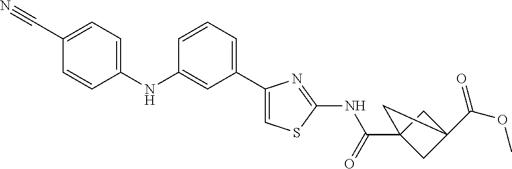 [SMILES: COC(=O)C12CC(C(=O)Nc3nc(-c4cccc(Nc5ccc(C#N)cc5)c4)cs3)(C1)C2] | 444.12 | 0.74 | 10.1 | 13.01 |
| 44 | 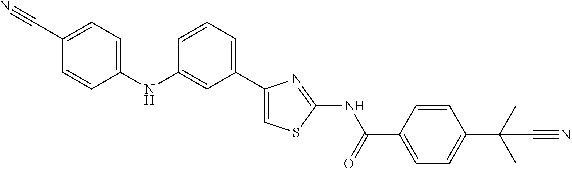 [SMILES: CC(C)(C#N)c1ccc(C(=O)Nc2nc(-c3cccc(Nc4ccc(C#N)cc4)c3)cs2)cc1] | 463.14 | 2.65 | 0.98 | >40 |
| 45 | 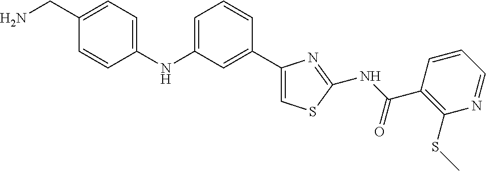 [SMILES: CSc1ncccc1C(=O)Nc1nc(-c2cccc(Nc3ccc(CN)cc3)c2)cs1] | 447.57 | 1.23 | 5.8 | 12.84 |
| 46 | 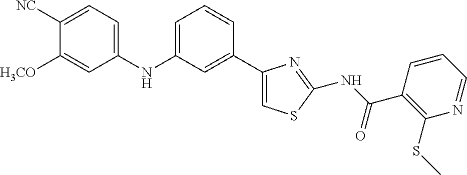 [SMILES: COc1cc(Nc2cccc(-c3csc(NC(=O)c4cccnc4SC)n3)c2)ccc1C#N] | 473.09 | 5.06 | 0.34 | >40 |
| 47 | 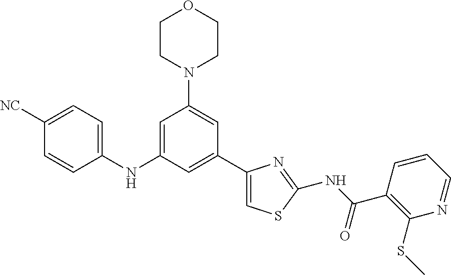 [SMILES: CSc1ncccc1C(=O)Nc1nc(-c2cc(Nc3ccc(C#N)cc3)cc(N3CCOCC3)c2)cs1] | 528.65 | 5.14 | 0.1 | >40 |
| 48 | 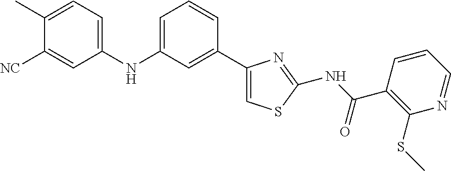 [SMILES: CSc1ncccc1C(=O)Nc1nc(-c2cccc(Nc3ccc(C)c(C#N)c3)c2)cs1] | 457.57 | 4.52 | 0.89 | >40 |
| 49 | 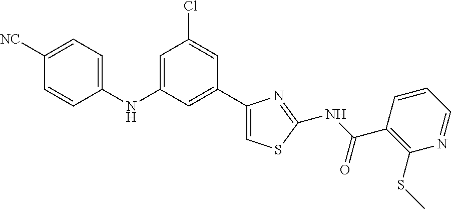 [SMILES: CSc1ncccc1C(=O)Nc1nc(-c2cc(Cl)cc(Nc3ccc(C#N)cc3)c2)cs1] | 477.99 | 5.84 | 0.21 | >40 |
| 50 | 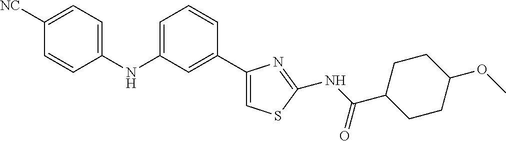 [SMILES: COC1CCC(C(=O)Nc2nc(-c3cccc(Nc4ccc(C#N)cc4)c3)cs2)CC1] | 432.54 | 4.53 | 0.1 | 12.4 |
| 51 | 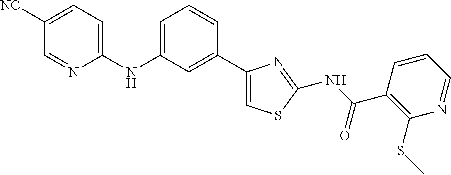 [SMILES: CSc1ncccc1C(=O)Nc1nc(-c2cccc(Nc3ccc(C#N)cn3)c2)cs1] | 444.53 | 2.16 | 4.84 | >40 |
| 52 | 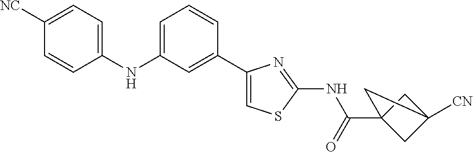 [SMILES: N#Cc1ccc(Nc2cccc(-c3csc(NC(=O)C45CC(C#N)(C4)C5)n3)c2)cc1] | 411.48 | 5.58 | 1.5 | 9 |

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having a structure represented by a formula: 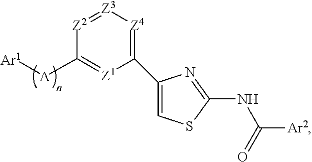
wherein n is selected from 0 and 1;
wherein A, when present, is selected from O, NH, N(C1-C4 alkyl), and N(Ar³);
wherein Ar³, when present, is phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein each of Z¹, Z², Z³, and Z⁴ is independently selected from N and CR¹⁰, provided that at least two of Z¹, Z², Z³, and Z⁴ are CR^{∘};
wherein each occurrence of R¹⁰, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², —SO₂R²³, and Cy¹;
wherein each occurrence of R²⁰, R²¹, R²², and R²³, when present, is independently selected from hydrogen and C1-C4 alkyl;
wherein Cy¹, when present, is selected from 5-membered heterocycle and 6-membered heterocycle, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, and C1-C4 aminoalkyl;
and either:
(a) wherein Ar¹ is selected from pyridinyl, piperazinyl, and 6-membered aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹, —CO₂R¹², —C(O)NR¹³, and —SO₂R¹⁴;
wherein each occurrence of R¹¹, R¹², R¹³, and R¹⁴, when present, is independently selected from hydrogen and C1-C4 alkyl; and
wherein Ar² is selected from 5-membered heterocycle, 6-membered heterocycle, 5-membered heteroaryl, and 6-membered heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸; and
wherein each occurrence of R¹⁵, R¹⁶, R¹⁷, and R¹⁸, when present, is independently selected from hydrogen and C1-C4 alkyl; or
(b) wherein Ar¹ is a structure represented by a formula: 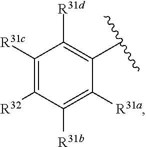
wherein each of R^{31a}, R^{31b}, R^{31c}, and R^{31d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴;
wherein R³² is selected from —CN and —CH₂NH₂; and
wherein Ar² is selected from 5-membered heterocycle, 6-membered heterocycle, 5-membered heteroaryl, 6-membered heteroaryl, 6-membered aryl, and bicyclo[1.1.1]pentanyl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸,
or a pharmaceutically acceptable salt thereof.

2. The compound of claim 1, wherein each of Z¹, Z², Z³, and Z⁴ is CR¹⁰.

3. The compound of claim 1, wherein each occurrence of R¹⁰, when present, is hydrogen.

4. The compound of claim 1, wherein Ar¹ is pyridinyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹¹, —CO₂R¹², —C(O)NR¹³, and —SO₂R¹⁴.

5. The compound of claim 1, wherein Ar¹ is pyridinyl para-substituted with a —CN group.

6. The compound of claim 1, wherein Ar¹ is 6-membered aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R¹, —CO₂R¹², —C(O)NR¹³, and —SO₂R¹⁴.

7. The compound of claim 1, wherein Ar¹ is 6-membered aryl para-substituted with a —CN group.

8. The compound of claim 1, wherein Ar¹ is a structure represented by a formula:

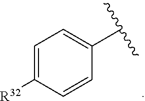

9. The compound of claim 1, wherein Ar² is a structure represented by a formula: 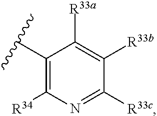
wherein each of R^{33a}, R^{33b}, and R^{33c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸; and
wherein R³⁴ is selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R⁵, —CO₂R¹⁶, —C(O)NR¹⁷, and —SO₂R¹⁸.

10. The compound of claim 1, wherein Ar² is a structure represented by a formula:

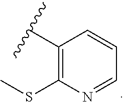 [SMILES: *c1cccnc1SC]

11. The compound of claim 1, wherein Ar¹ is a structure represented by a formula: 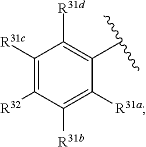
and
wherein Ar² is selected from 5-membered heterocycle, 6-membered heterocycle, 5-membered heteroaryl, 6-membered heteroaryl, 6-membered aryl, and bicyclo[1.1.1]pentanyl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 thioalkyl, C1-C4 thiohaloalkyl, C1-C4 haloalkoxy, C1-C4 alkoxy, C1-C4 alkylamino, (C1-C4)(C1-C4) dialkylamino, C1-C4 aminoalkyl, —C(O)R¹⁵, —CO₂R¹⁶, —C(O)NR¹⁷, and —SO₂R¹⁸.

12. The compound of claim 1, wherein the compound has a structure represented by a formula:

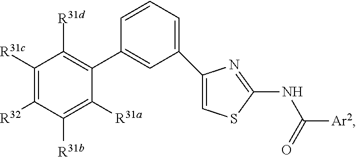

13. The compound of claim 1, wherein the compound has a structure represented by a formula: 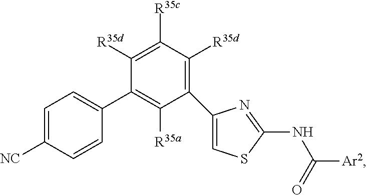
wherein each of R^{35a}, R^{35b}, R^{35c}, and R^{35d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³.

14. The compound of claim 1, wherein the compound has a structure represented by a formula:

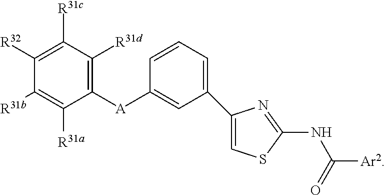

15. The compound of claim 1, wherein the compound has a structure represented by a formula: 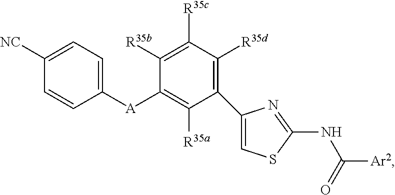
wherein each of R^{35a}, R^{35b}, R^{35c}, and R^{35d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³.

16. The compound of claim 1, wherein the compound is selected from:

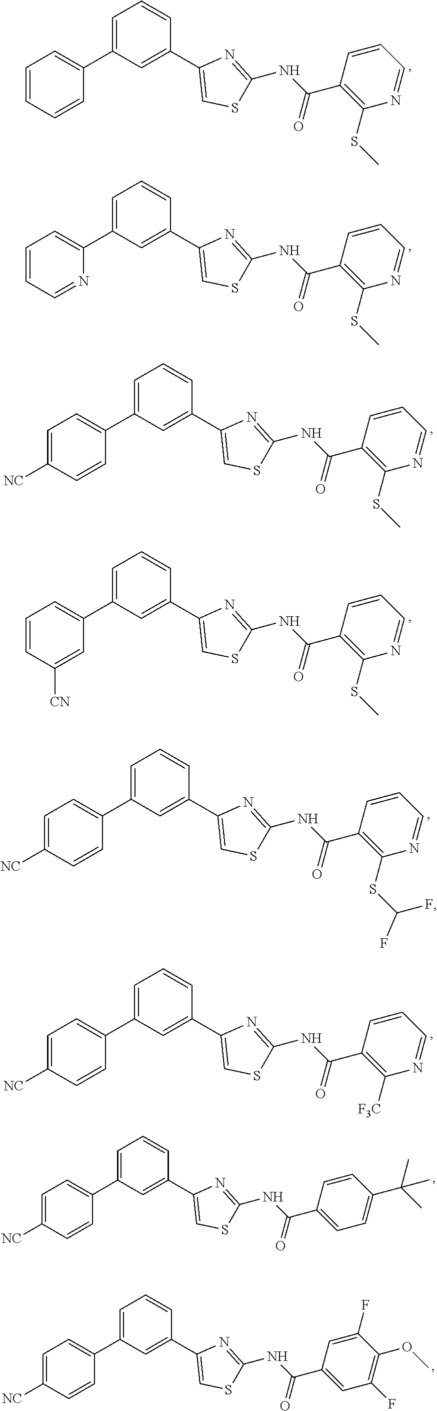

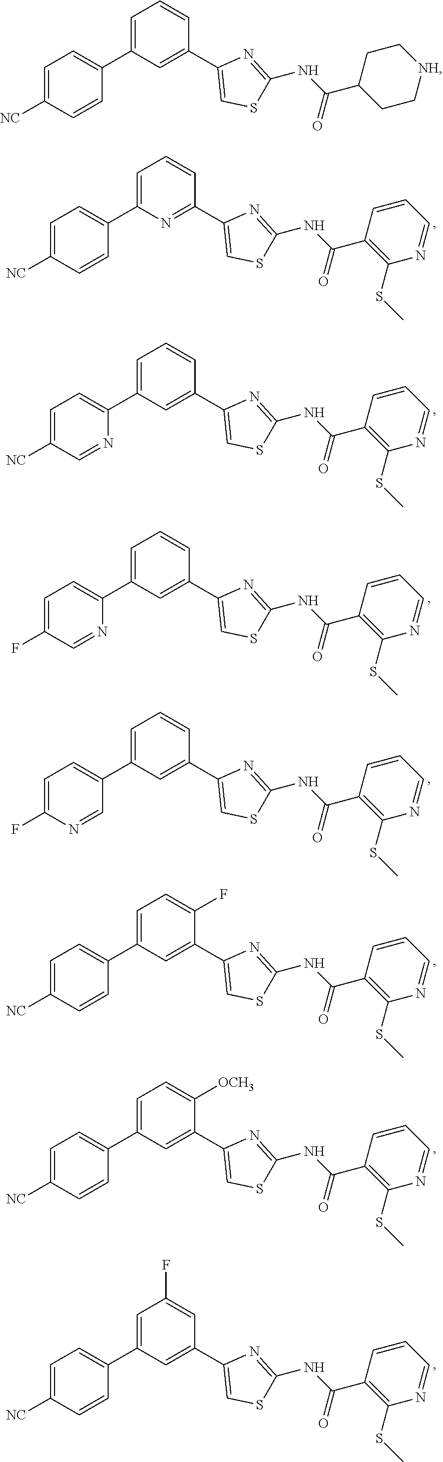

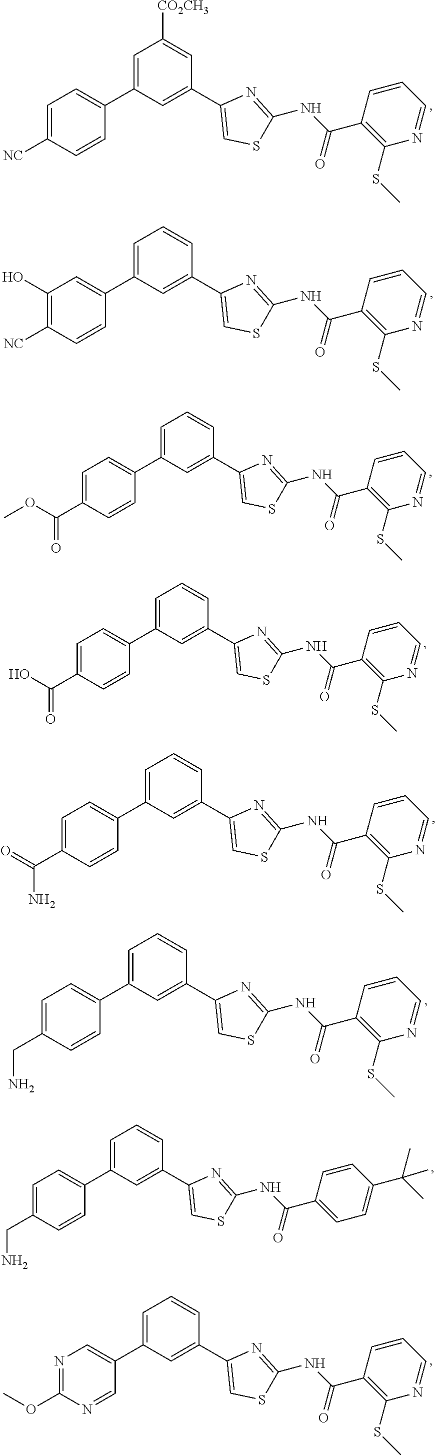

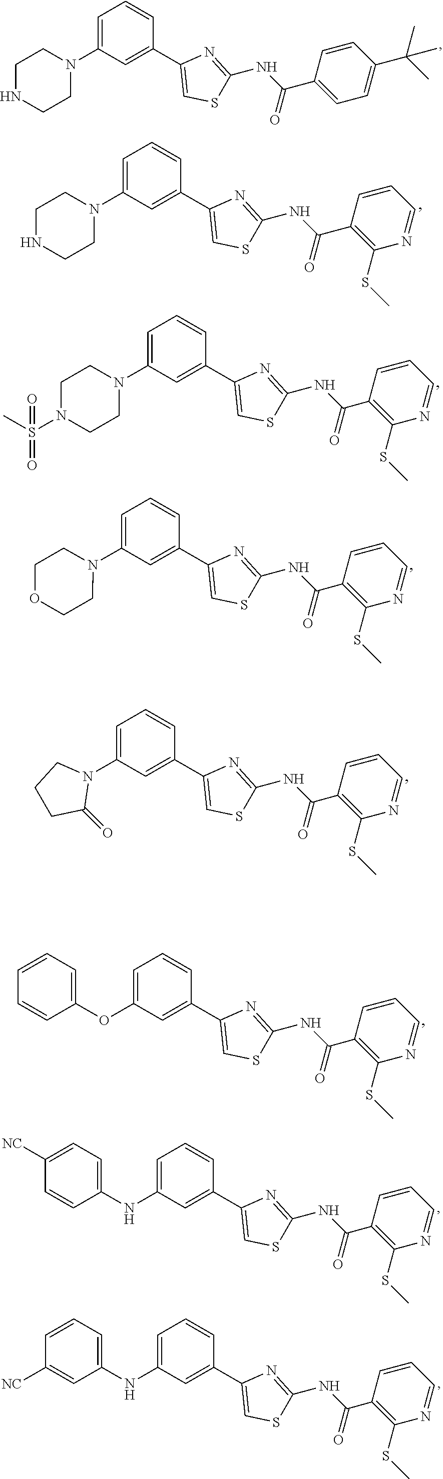

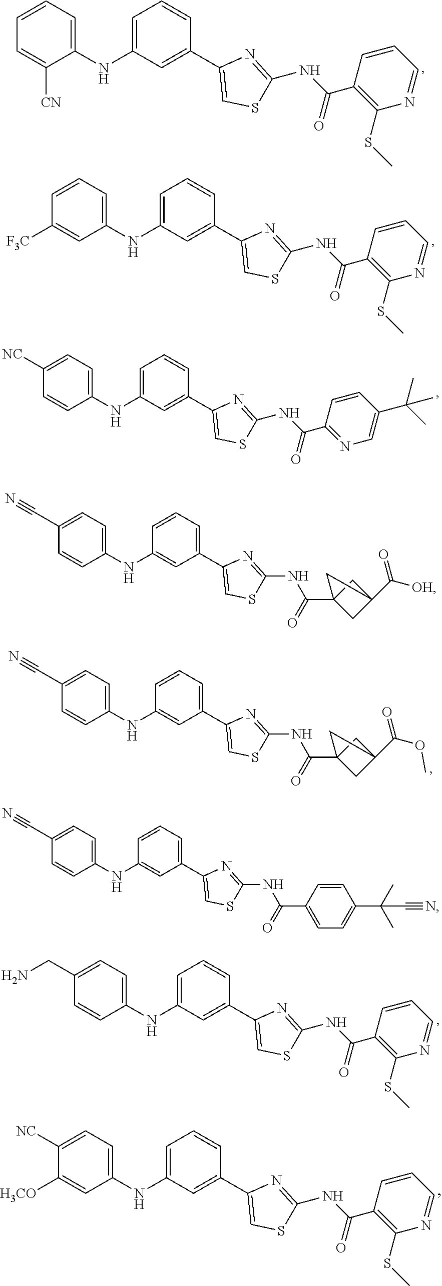

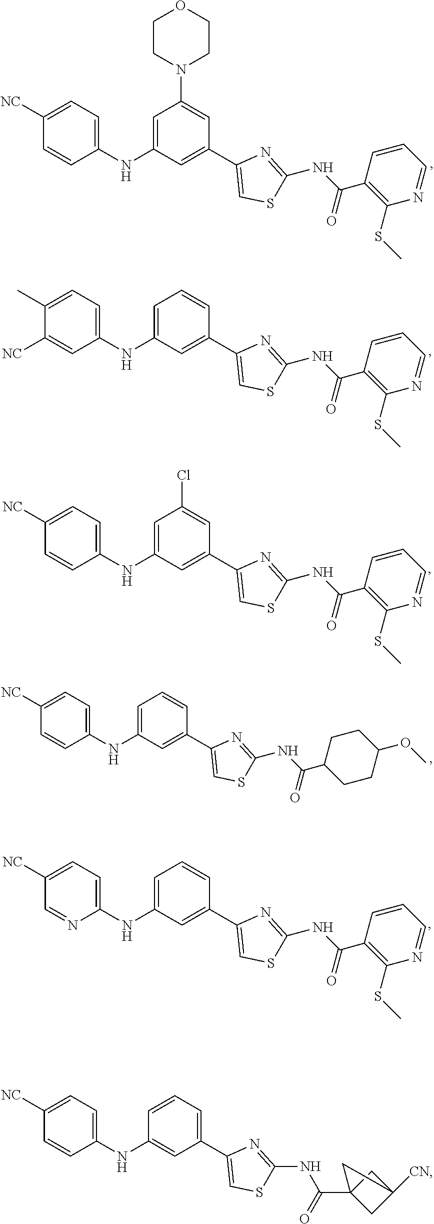 [SMILES: CSc1ncccc1C(=O)Nc1nc(-c2cc(Nc3ccc(C#N)cc3)cc(N3CCOCC3)c2)cs1 ; CSc1ncccc1C(=O)Nc1nc(-c2cccc(Nc3ccc(C)c(C#N)c3)c2)cs1 ; CSc1ncccc1C(=O)Nc1nc(-c2cc(Cl)cc(Nc3ccc(C#N)cc3)c2)cs1 ; COC1CCC(C(=O)Nc2nc(-c3cccc(Nc4ccc(C#N)cc4)c3)cs2)CC1 ; CSc1ncccc1C(=O)Nc1nc(-c2cccc(Nc3ccc(C#N)cn3)c2)cs1 ; N#Cc1ccc(Nc2cccc(-c3csc(NC(=O)C45CC(C#N)(C4)C5)n3)c2)cc1]

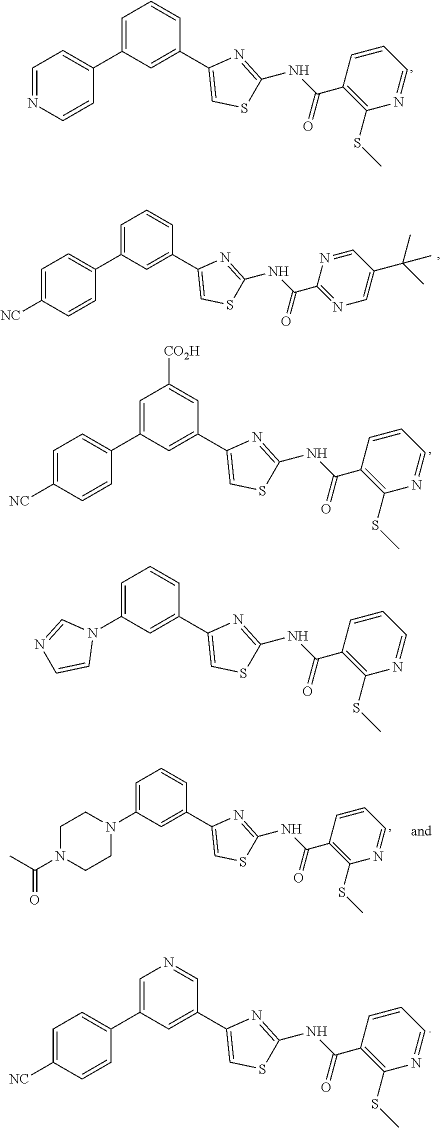

17. A pharmaceutical composition comprising a therapeutically effective amount of the compound of claim 1 and a pharmaceutically acceptable carrier.

18. A method for treating a viral infection in a subject, the method comprising administering to the subject an effective amount of the compound of claim 1.

19. The method of claim 18, wherein the viral infection is selected from human immunodeficiency virus (HIV), human papillomavirus (HPV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, Chikungunya virus (CHIKV), Venezuelan equine encephalitis (VEEV), Eastern equine encephalitis (EEEV), Western equine encephalitis (WEEV), dengue (DENV), influenza, West Nile virus (WNV), and zika (ZIKV).

20. The method of claim 18, wherein the viral infection is CHIKV.
